(19) United States
(12) Patent Application Publication
(10) Pub. No.: US 2003/0078192 A1
(43) Pub. Date: Apr. 24, 2003

(54) Combinatorial protein domains

(76) Inventors: Gregory Paul Winter, Cambridge (GB); Lutz Riechmann, Cambridge (GB)

Correspondence Address: PALMER & DODGE, LLP, KATHLEEN M. WILLIAMS, 111 HUNTINGTON AVENUE, BOSTON, MA, 02199, US

(21) Appl. No.: 10/119,556

(22) Filed: Apr. 10, 2002

Related U.S. Application Data
(63) Continuation-in-part of application No. 09/938,945, filed on Aug. 24, 2001.
(63) Continuation-in-part of application No. PCT/GB01/00445, filed on Feb. 2, 2001.
(60) Provisional application No. .

(30) Foreign Application Priority Data
Feb. 3, 2000 (GB) 0002492.7
Aug. 7, 2000 (GB) 0019362.3
Jul. 3, 2000 (GB) 0016346.9

(51) Int. Cl. A61K 38/16

(52) U.S. Cl. 514/2; 514/12

(57) ABSTRACT

The invention relates to a pharmaceutical composition comprising a chimeric, folded protein domain comprising two or more sequence segments from parent amino acid sequences that are not homologous. The invention more particularly relates to compositions comprising a chimeric, folded protein domain comprising two or more sequence segments wherein each of the sequence segments: is not designed or selected to consist solely of a single complete protein structural element and is not designed or selected to consist solely of an entire protein domain; and, in isolation, shows no significant folding at the melting temperature of the chimeric protein. The invention also relates to methods for the selection of such protein domains, and to methods of raising an immune response using such domains, and preferably to chimeric domains that display conformational B cell epitopes of at least one of their parent amino acid sequences.

This application is a continuation-in-part of PCT/GB01/00445, filed Feb. 2, 2001, which claims the priority of U.K. Patent Application 0002492.7, filed Feb. 3, 2000, U.S. Provisional Application No. 60/180,326, filed Feb. 4, 2000, U.K. Patent Application 0016346.9, filed Jul. 3, 2000, and U.K Patent Application 0019362.3, filed Aug. 7, 2000. This application also claims the priority of U.S. Provisional Application No. 60/228,078, filed Aug. 25, 2000, and U.S. Utility application Ser. No. 09/938,945, filed Aug. 24, 2001.

### BACKGROUND OF THE INVENTION

The de novo design of proteins is typically based on structure predictions of predetermined amino acid sequences (Hecht 1994, Sauer 1996, Regan 1998). Partial randomisation is often introduced to allow for imperfection in the prediction algorithms. Resulting repertoires are screened or selected for stably folded structures. This approach has been successful for the design of helical structures like four helix bundles with stable and compact structures exhibiting free energies of unfolding of about 4 kcal/mol (Kamtekar et al. 1993). More problematic has been the design of β-sheet proteins, where even the most recent attempts fell well short of natural β-sheet proteins with respect to stability (Quinn et al. 1994, Kortemme et al. 1998, Alba et al. 1999). Problems in the design of β-sheet structures are related to their dependence on backbone hydrogen bonds between different secondary structure elements, which are less well understood than the principles of helix formation (Hecht 1994). Repertoires of random protein sequences have also been screened for the occurrence of folded proteins. About 1% of members in a random library of Glu, Leu, Arg rich proteins exhibited some helix formation and cooperative unfolding but were unstable (Davidson & Sauer 1994).

Recently, new strategies to select stably folded proteins from repertoires of phage displayed proteins based on their resistance to proteolytic degradation have been used to improve the stability of natural proteins (Kristensen & Winter 1997, Sieber et al. 1998, Finucane et al. 1999). Proteolytic degradation is usually restricted to unfolded proteins or highly flexible regions of folded proteins. Folded proteins are mostly resistant to proteases, because the proteolytic cleavage requires the polypeptide chain to adapt to the specific stereochemistry of the protease active site, and therefore to be flexible, accessible and capable of local unfolding (Hubbard et al. 1994, Fontana et al. 1997). These methods have only been described for selection of proteins with point mutations; no element of combining sequences from different proteins is involved.

The oligomerization of microgenes based on secondary structure elements was described by Shiba et al. (1997, Proc. Natl. Acad. Sci. U.S.A. 94: 3805-3810). There was no selection of the resulting proteins and no evidence for the creation of folded domains in that publication.

A theoretical approach to protein evolution via combinatorial rearrangement of defined, complete structural elements has been described (Bogarad & Deem 1999). The authors predict, using statistical algorithms, that rearrangement of a number of structural elements (such as helices, strands, loops, turns and others) will result in the generation of novel protein functions more rapidly than evolution by point mutation strategies alone. However, no allowance for the context dependence of structure is made, nor is any reference made to partial structural domains which possess no structural identity in isolation. Although some (rare) sequences will form structures in isolation, others can adopt a different structure in a different environment as evidenced by structural rearrangements following cleavage of some polypeptides by protease or on ligand binding. It is therefore not easy to define a structural element except in the context of the three-dimensional structure of the protein in which it is embedded, and it is this definition that we have adopted here. Furthermore the Bogarad & Deem paper does not show that it will be possible to undertake this process in vitro, or indicate exactly how to undertake such experiments.

### SUMMARY OF THE INVENTION

The invention is based on a strategy for the creation and selection of novel protein domains which are capable of forming stably folded structures, and thus of identifying novel protein structural and functional elements.

The invention is based upon the recognition that because the structure of a “structural” element is dependent on context, single structural elements taken from one protein and appended to single structural elements taken from a second protein will not necessarily retain their original structure. Accordingly the inventors have not sought to restrict the segments to single complete structural elements. Furthermore the use of parts of structural elements can provide new structures that are not simply due to juxtaposition of existing structural elements, and the use of segments comprising multiple structural elements (and making packing interactions with each other) would be expected to be more stable than single structural elements, and more likely to comprise a significant “nugget” of structure in the chimeric domain.

As used herein, the term “structural element” refers to a sequence of amino acids that, within the context of a parent polypeptide, assumes a stable secondary structure. “Structural elements” include α-helices, β-sheets (parallel, antiparallel, or mixed), 3₁₀ helices, pi-helices, loops and β-turns (Types I, II, or III).

As used herein, the term “stable secondary structure” refers to the three dimensional arrangement assumed by an amino acid sequence in which there is a pattern of hydrogen bonds between amino acids that place the region of amino acid sequence having that pattern into a particular structural conformation in the context of a parent polypeptide. Structural conformations of “stable secondary structure” as the term is used herein include α-helices, β-sheets (parallel, antiparallel, or mixed), 3₁₀ helices, pi-helices, loops and β-turns. By “stable” secondary structure is meant that the secondary structure is substantially retained (i.e., the at least 50% of the molecules in a population retain the hydrogen bonds characteristic of that structure, preferably at least 60%, 70%, or 80%, and more preferably at least 85%, 90% or more) under normal physiological conditions of temperature, pH and salt (e.g., 32° to 39° C., pH 6.0 to pH 8.0, and isotonic salt) in the context of a parent polypeptide. The determination of secondary structure possessed by a given amino acid sequence in the context of a parent polypeptide is determined by NMR or X-ray crystallography according to methods known in the art.

As used herein, an α-helix is characterized by hydrogen bonding between the C═O of amino acid n and the N—H of amino acid n+4 in a given sequence. An α-helix has 3.6 amino acid residues per helical turn. The symbols φ and ψ are used in the art to describe the angles of rotation about the N—Cα and Cα—C′ bonds of each peptide unit, respectively. In an α-helix, the angle φ is approximately −60° and the angle ψ is approximately −50°. One helical turn is considered to establish an α-helix as the term is used herein. Note however, that a single turn does not necessarily constitute a “complete structural element,” as that term is defined herein below, unless that single turn is not contiguous with one or more additional a-helical turns in the context of the parent polypeptide.

A β-sheet is characterized by the presence of two or more β-strands of at least 5 amino acids that are in an extended conformation with respect to each other (about 3.5/per residue); the β-strands are aligned either parallel or antiparallel to each other and backbone C═O and N—H groups form hydrogen bonds between the strands to form the β-sheet. Alternating Cα carbons in the amino acid sequence of a β-sheet will be above and below the average plane of the sheet. A β-sheet is established by one grouping of two β-strands hydrogen bonded in a parallel or antiparallel orientation to each other. A single such grouping does not, however, necessarily constitute a “complete structural element,” as that term is defined herein below, unless that single grouping does not comprise one or more additional β-strands in the context of the parent polypeptide.

A β-turn is a short secondary structure element characterized by a hydrogen bond between the C═O group of residue n and the N—H group of residue n+3. A β-turn element often introduces a 180° turn in the polypeptide structure. Because a β-turn has several unsaturated backbone hydrogen bond donors and acceptors, it is polar, and is usually found near the surface of the protein. Turns are classified into three types according to the angles of rotation, φ and ψ, of the two central residues (residue 2 and 3). The main types are Type I and Type II which have mirror images in Types I′ and II′. Proline is very common in β-turns, as it always has the correct φ angle (−60) and it has one less unsaturated hydrogen donor.

In a Type I turn, the backbone dihedral angles of residue are (—60, —30) and (—90, 0) of residues n+1 and n+2, respectively. Proline is often found in position n+1 in Type I turns as its φ angle is restricted to −60 and its imide nitrogen does not require a hydrogen bond.

In a Type II turn the backbone dihedral residue angles are (—60, 120) and (80, 0) for residues n+1 and n+2, respectively. Glycine is favored in the n+1 position in the type II′ as it requires a positive (left-handed)+value.

A Type III turn is a single turn of right-handed (III) or left-handed (III′) 3₁₀ helix (see below). The backbone dihedral residue angles are (—60, —30) and (—60, —30) for residues n+1 and n+2, respectively of the classical type III turn.

A 3₁₀ helix is characterized by having 3 residues per turn of the helix, which is tighter than an α-helix. 3₁₀ helices tend to be shorter (i.e., have fewer repeats) than a-helices, and are often found at the C-terminus of a longer α-helix. One turn of the helix establishes a 3₁₀ helix, but single turn of 3₁₀ helix does not necessarily constitute a “complete structural element,” as that term is defined herein below, unless that single turn of 3₁₀ helix does not comprise one or more additional β-strands in the context of the parent polypeptide.

A pi helix has repeating hydrogen bonds in which the C═O of residue n hydrogen bonds to the N—H of residue n+5. Pi helices are relatively rare, most often found a the termini of longer α-helices, and seldom comprise more than a few n, n+5 hydrogen bond iterations. A pi helix is established by a single turn of pi helix, but does not constitute a “complete structural element” as the term is used herein unless that single turn is not contiguous with one or more additional turns of pi helix.

A loop structure is defined not by its own structure, but by its position between other structural elements. That is, a loop structure does not define a structural element other than by forming a region without a regular hydrogen bonding arrangement, contiguously two regions that do have regular hydrogen bonding arrangement. By “regular hydrogen bonding arrangement” is meant a hydrogen bonding arrangement as seen between backbone C═O and N—H groups in a secondary structure consisting of an α-helix, a β-sheet (parallel, antiparallel, or mixed), a 3₁₀ helix, a pi-helix, and a β-turn (Type I, II, or III).

As used herein, the term “complete structural element” or “complete protein structural element” refers to a sequence of amino acids that, in the context of a parent polypeptide, comprises all amino acids of the single structural element defined by that sequence. For example, if amino acids 40 to 70 in a hypothetical 100 amino acid parent polypeptide define an α-helix as determined by NMR, amino acids 40-70 are a “complete structural element.” A sequence comprising fewer than (i.e., at least 1 amino acid fewer) all of the amino acids defining such a structural element is not a “complete structural element.” That is, a sequence comprising, for example, amino acids 45 to 65, or 40-69 or even 41 to 70 would not be a “complete structural element as defined herein. Such a sequence would be “less than a complete structural element.”

Similarly, a sequence comprising more than (i.e., at least one amino acid more) the amino acids defining a structural element in the context of a parent polypeptide is not a “complete structural element,” but is “more than a complete structural element.” Using the same hypothetical parent polypeptide, a sequence comprising amino acids 35 to 80, or even 39 to 70 or 40 to 71 is “more than a complete structural element.”

As used herein, the term “single and complete protein structural element” refers to a complete protein structural element that is not linked to another complete protein structural element.

The present invention exploits protein evolution by juxtaposition of sequence segments. “Sequence segments”, as referred to herein, are amino acid sequences which consist of more amino acid residues than or fewer amino acid residues than define a complete structural element, and consist of less than a complete protein domains. That is, “sequence segments” as referred to herein are amino acid sequences which are not designed or selected to consist solely of single and complete protein structural elements; and are not designed or selected to consist of a complete protein domain. The present invention is thus directed to the juxtaposition of blocks of more than one structural element or to the creation of novel structural elements by the juxtaposition of sequences which in their parent environments, possess no discrete and complete structure as measured by NMR, microcalorimetry or X-ray crystallography. The invention is therefore not directed to the juxtaposition of discrete and single elements of structure found in naturally-occurring or synthetic proteins.

A sequence element in isolation from its parent polypeptide has no discrete or complete structure if the free energy of folding is 1.5 kcal/mol or less, as measured by microcalorimetry.

For a sequence element in the context of a parent polypeptide, structure is determined by NMR or X-ray crystallography. A sequence element in the context of its parent polypeptide has no discrete or complete structure if the NMR spectra or X-ray crystal structure show that the sequence segment comprises 1 amino acid less than or more than a complete structural element (e.g., less than or more than the full length of an α helical portion of the polypeptide), but preferably 5, 10 or more amino acids less than or more than the full length of a structural element, or comprises at least part of more than one structural element.

Therefore, a “sequence segment” is an amino acid sequence that, in its parent environment, comprises no complete and discrete protein domain and is not encoded by one or more complete natural exons. Moreover, a “sequence segment”, in its parent environment, is either part of a structural element or, advantageously, is longer than a structural element, and does not form one or more discrete structural elements. The sequence segment in isolation shows no significant folding at the melting temperature of the chimeric protein (i.e., the free energy of folding is less than 1.5 kcal/mol); in other words, it possesses no independent structure in isolated form.

As used herein, the term “protein domain” refers to a combination of at least two polypeptide segments that together fold to form a stable tertiary structure. In isolation, the polypeptide segments comprising a “protein domain” according to the invention do not fold and do not define a single structural element in the parent protein.

As used herein, the term “tertiary structure” refers to the folds of a polypeptide that define its overall three dimensional shape. “Tertiary structure” generally refers to the interactions between amino acids that are distant from one another in the primary amino acid sequence. Most often, tertiary structure results from the folding of regions having regular secondary structure with other such regions or with globular regions that do not have regular secondary structure. Tertiary structure is frequently, but not necessarily, stabilized by disulfide bonds. As used herein, the “term “stable tertiary structure” or “stably folded structure” refers to a tertiary structure that has a free energy of folding of 1.6 kcal/mol or higher. The stability of a tertiary structure can also be expressed in terms of resistance to proteolysis as defined herein below.

As used herein, the term “resistant to proteolysis” means that digestion with a given protease results in less than a 20% decrease in the amount of intact protein, relative to the amount determined prior to proteolysis. That is, a chimeric protein domain will retain at least 80% of its full-length molecules after digestion with a protease. In practice, it is convenient to measure protease resistance in a population of phage displaying the chimeric polypeptide according to the invention. In that instance, a population of chimeric polypeptide-displaying phage that retains at least 80% of its ability to bind a ligand through an N-terminal tag linked to the chimeric protein domain after digestion is “resistant to proteolysis” as the term is used herein. Exemplary digestion conditions are set forth in the examples herein below.

The “parent environment” of the sequence segment is the protein or polypeptide from which that segment is taken, in its folded state. This can be a natural protein, or an artificial polypeptide or protein. Preferably, the sequence segment is taken from an amino acid sequence which is longer than the sequence segment itself.

As used herein, the term “complete protein domain” refers to a protein domain which in isolation has a stable tertiary structure. A “complete protein domain” can be fused to a folded chimeric protein domain according to the invention, thereby providing, for example, a means to select the chimeric folded protein domain. Non-limiting examples of “complete protein domains” include domains that confer binding to a ligand, recognition by an antibody, or catalytic activity.

As used herein, the term “non-homologous” means that parent amino acid sequences from which sequence segments are derived have less than 50% sequence similarity to each other. Preferably, parent amino acid sequences from which sequence segments useful according to the invention are derived will have less than 40%, 30%, 20% or even lower amino acid similarity to each other.

As used herein, the term “sequence similarity” refers to relationships between two or more polypeptide sequences as determined by comparing the sequences. Amino acid sequence “similarity” between two sequences is determined from an optimal global alignment of the sequences being compared. An optimal global alignment is achieved using, for example, the Needleman—Wunsch algorithm (Needleman and Wunsch, 1970, J. Mol. Biol. 48:443-453). “Identity” means that an amino acid or nucleotide at a particular position in a first polypeptide or polynucleotide is identical to a corresponding amino acid or nucleotide in a second polypeptide or polynucleotide that is in an optimal global alignment with the first polypeptide or polynucleotide. In contrast to identity, “similarity” encompasses amino acids that are conservative substitutions. A “conservative” substitution is any substitution that has a positive score in the blosum62 substitution matrix (Hentikoff and Hentikoff, 1992, Proc. Natl. Acad. Sci. USA 89: 10915-10919). By the statement “sequence A is n % similar to sequence B” is meant that n % of the positions of an optimal global alignment between sequences A and B consist of conservative substitutions. For the purposes of this invention, optimal global alignments are achieved using the following parameters in the Needleman-Wunsch alignment algorithm:

For polypeptides:

Substitution matrix: blosum62.

Gap scoring function: —A—B*LG, where A=11 (the gap penalty), B=1 (the gap length penalty) and LG is the length of the gap.

For nucleotide sequences:

Substitution matrix: 10 for matches, 0 for mismatches.

Gap scoring function: —A—B*LG where A=50 (the gap penalty), B=3 (the gap length penalty) and LG is the length of the gap.

Typical conservative substitutions are among Ala, Val, Leu and Ile; among Ser and Thr; among the acidic residues Asp and Glu; among Asn and Gln; and among the basic residues Lys and Arg; or aromatic residues Phe and Tyr. In calculating the degree (usually a percentage) of similarity between two polypeptide sequences, one considers the number of positions at which identity or similarity is observed between corresponding amino acid residues in the two polypeptide sequences in relation to the entire lengths of the two molecules being compared.

According to the present invention in a first configuration, the combinatorial rearrangement of protein sequence segments permits the selection of novel folded protein domains from combinatorial repertoires.

In a first aspect, therefore, the invention provides a chimeric folded protein domain, selected from a repertoire of chimeric proteins, the chimeric protein domain comprising two or more sequence segments derived from parent amino acid sequences that are not homologous.

A polypeptide or amino acid sequence “derived from” a designated nucleic acid sequence refers to a polypeptide having an amino acid sequence identical to that of a polypeptide encoded in the designated sequence, or identical to a portion of the designated sequence, wherein the portion consists of at least 15 amino acids, preferably at least 20 amino acids or more This terminology also includes a polypeptide expressed from a designated nucleic acid sequence. An amino acid or polypeptide sequence “derived from” another sequence can include one or more amino acid insertions, deletions or substitutions relative to the sequence it is “derived from.”

A chimeric polypeptide “derived from” a repertoire of chimeric proteins is one that was selected from such a repertoire.

Preferably, the parent amino acid sequences are derived from protein domains. The parent amino acid sequences can be natural, semi-synthetic or synthetic in origin. They can be prepared by expression from genes or assembled by chemical synthesis.

Advantageously, the amino acid sequence segments are derived from proteins. In an advantageous embodiment, the proteins are selected from the group consisting of a naturally occurring protein, an engineered protein, a protein with a known binding activity, a protein with a known binding activity for an organic compound, a protein with a known binding activity for a peptide or polypeptide, a protein with a known binding activity for a carbohydrate, a protein with a known binding activity for a nucleic acid, a known binding activity for a hapten, a protein with a known binding activity for a steroid, a protein with a known binding activity for an inorganic compound, and a protein with an enzymatic activity.

As used herein, the term “B cell epitope” refers to a peptide, including a peptide comprised in a larger protein, which is recognized by and binds to a B cell immunoglobulin receptor, and which participates in the induction of antibody production by the B cell. B cell epitopes can be identified by methods known in the art, as set forth, for example, by Caton et al., 1982, Cell 31: 417-427.

As used herein, the term “T cell epitope” refers to a peptide, including a peptide comprised in a larger protein, which associates with MHC self antigens, is recognized by a T cell, and which functionally activates the T cell. Association with MHC self antigens, T cell binding and T cell activation can be measured according to methods known in the art.

As used herein, the term “engineered protein” refers to a non-naturally-occurring polypeptide. The term encompasses, for example, a polypeptide that comprises one or more changes, including additions, deletions or substitutions, relative to a naturally occurring polypeptide, wherein such changes were introduced by recombinant DNA techniques. The term also encompasses a polypeptide that comprises an amino acid sequence generated by man, and a chimeric polypeptide. Those skilled in the art can readily generate engineered proteins useful according to this aspect of the invention.

As used herein, the term “binding activity” refers to the property of a given polypeptide or polypeptide domain whereby it physically associates with one or more other moieties. By “physically associate” is meant an interaction with a dissociation constant (K_{d}) of less than 10⁻⁵ M, preferably less than 10⁻⁶ M, less than 10⁻⁷ M, less than 10⁻⁸ M, less than 10⁻⁹ M, or less than 10⁻¹⁰ M or lower.

As used herein, a “known binding activity” is a binding activity as defined herein, possessed by a polypeptide or polypeptide domain, which activity is known to those skilled in the field of protein interactions.

As used herein, “amino acid” includes the 20 naturally-occurring amino acids, as well as non-naturally occurring amino acids and modified amino acids, such as tagged or labelled amino acids. As used herein, the term “protein” refers to a polymer in which the monomers are amino acids and are joined together through peptide or disulphide bonds. Preferably, “protein” refers to a full-length naturally-occurring amino acid chain or a fragment thereof, such as a selected region of the polypeptide that is of interest in a binding interaction, or a synthetic amino acid chain, or a combination thereof.

As used herein, the term “non-naturally occurring amino acid” refers to an amino acid that is not found in proteins in nature. Non-limiting examples include L-tert-leucine, L-homophenylalanine, D-homophenylalanine, D-methionine, halogenated D and L-phenylalanines, tyrosines, and tryptophans, D-2-aminopimelic acid, L-2-aminopimelic acid, L-phosphinothricin, L-2-aminohexanoic acid and 15-N substituted amino acids.

As used herein, the term “amino acid having a label or tag” refers to a naturally occurring or non-naturally occurring amino acid that bears a detectable marker. Detectable markers include, for example, radioisotopes (e.g., ¹²⁵I, ³⁵S), fluorescent or luminescent groups, biotin, haptens, antigens and enzymes.

The sequence segments can be combined, in the chimeric protein domain, by any appropriate means. Typically, the segments will be combined by recombinant DNA techniques and will thus be joined, in the recombinant protein, by peptide bonds. In alternative embodiments, the segments can be synthesised separately and subsequently joined. This can be achieved using covalent linkage, for instance peptide bonds, ester bonds or disulphide bonds, or non-covalent linkage. Advantageously, sequence segments according to the invention comprise one or more reaction groups for covalent or non-covalent linkage. For example, linkers capable of associating non-covalently, such as biotin/streptavidin, can be incorporated into the sequence segments to effect non-covalent linkage.

As used herein, the term “reaction group” refers to a moiety that permits the linkage of one sequence segment to another sequence segment. The term “reaction group for covalent linkage” refers to a chemical group that permits covalent linkage between sequence segments. Reaction groups for covalent linkage include, for example, an amine, a carboxyl, a thiol, maleimide, an azido, an isocyanato, an isothiocyanato, an acyl halide, a succinimidyl ester, or a sulfosuccinimidyl ester, among others. The term “reaction group for non-covalent linkage” refers to a moiety that permits non-covalent linkage between sequence segments, examples of which include biotin, digoxygenin, avidin, streptavidin, and antibodies or Fabs, among others.

As used herein, the term “repertoire” refers to a plurality of different (i.e., by one amino acid or more) chimeric polypeptide or protein domains. The repertoire from which the chimeric protein domain is derived can be of substantially any size. Preferably, the repertoire comprises at least 10,000 individual protein domains; advantageously it comprises at least 1,000,000 protein domains; and most preferably, at least 100,000,000 protein domains. The sequence segments useful according to the invention can be any appropriate number of amino acids in length such that the combined length of the segments represents the length of a complete domain, which domains vary from as little as about 35 residues to several hundred residues in length.

In an advantageous aspect, the parent amino acid sequences are derived from the open reading frames (ORFs) of a genome or part thereof:

(a) wherein said reading frames are the natural reading frame of the genes; or

(b) wherein said reading frames are not the natural reading frame of the genes.

Sequences can thus be derived from ORFs present in a whole or substantially whole genome of an organism, or a part thereof, such as a group or family of genes, whether related by structure, function or evolution, or not related. The part of the genome can also consist of a single gene.

Sequences can moreover be derived from two or more genomes, from organisms of related or unrelated species.

The protein domains according to the invention are capable of folding due to the combination of two or more polypeptide segments which, in isolation, do not fold and do not define a single structural element in the parent protein.

Advantageously, the protein domains according to the invention are selected according to their resistance to proteolysis. This provides a useful means to isolate candidate domains from libraries; a selection procedure can be configured such that only proteolysis-resistant domains are selected from the libraries. Preferably, the proteolysis is carried out by exposure to a protease, such as thermolysin.

In a preferred embodiment, the protein domains according to the invention can be selected according to their activity. This can for example be a binding activity, for example in the case of immunoglobulin-type domains, or an enzymatic activity in the case of enzyme domains. Alternatively the protein domain can have the capacity to bind antibodies directed against the parent protein. Moreover, a screen for activity can be performed in addition to a selection on the basis of folding as determined by protease resistance. Such an approach is particularly advantageous where an initial selection on the basis of activity would be difficult or impossible to perform.

Moreover the invention concerns the juxtaposition of sequence fragments derived from non-homologous domains which share a similar polypeptide fold for at least part of the structure. We have observed that, in selections of protein domains according to the invention, sequence segments derived from parental protein domains having similar folds for at least part of their structures are juxtaposed in some of the novel chimeric proteins. Accordingly, the present invention provides a chimeric protein according to the first aspect of the invention, wherein the sequence segments originate from parent domains with similar polypeptide folds in at least part of the structure.

It has further been observed that, in selections of protein domains according to the invention, sequence segments derived from parental protein domains having entirely different folds for at least part of their structures are juxtaposed in other novel structures. Accordingly, the present invention provides a chimeric protein domain comprising two or more sequence segments derived from parent amino acid sequences, wherein the sequence segments originate from parent domains with different polypeptide folds in at least part of the structure.

Moreover, in selections of protein domains according to the first configuration of the invention, sequence segments derived from the same protein domain can be observed to be juxtaposed to form novel structures. In some cases said sequence segments can comprise regions in common leading to a duplication of sequence in the chimeric protein. However the common region does not consist solely of one or more complete protein structural elements. Therefore it appears that duplication of amino acid segments or parts thereof, without regard to the presence of solely one or more complete structural elements, can lead to the formation of stably folded structures. Such duplications comprise a second configuration of the invention.

As used herein, “region of common sequence” or “common region” refers to sequence segments in a chimeric polypeptide of the invention which share sequence similarity or are of a similar fold. In this context, sequence similarity refers to contiguous stretches of identical sequence of at least 10 amino acid residues; preferably of at least 20 contiguous amino acid residues.

According to the second configuration of the invention, the combination of segments from homologous proteins, leading to equivalent regions from these homologous proteins being brought together in the same chimeric protein, would also be expected to lead to the creation of stably folded structures. Regions, which are equivalent in homologous proteins, are identified by an alignment of their amino acid sequences. Indeed it is even possible to combine segments from non-homologous proteins which share a common fold (vide supra), to create stably folded chimeric proteins according to the second configuration of the invention from segments comprising a common region of the common fold in the parent proteins.

Stably folded structures based on duplication of amino acid segments have been created as a product of the random shuffling of amino acid segments and were selected through proteolytic selection because of their stability. Duplication or indeed multimerisation performed in other non-random ways have been previously reported, including for example by Hardies et al. 1979 and Fire & Xu 1995. The inventors envisage that said methods for duplication and multimerisation can also be used for the duplication or multimerisation of amino acid segments to create novel and stably folded domains under the second configuration of the invention. Such stable domains can be selected and screened for in ways identical or similar to those in case of chimeric domains derived from combinatorial shuffling.

Protein domains according to both configurations of the present invention can be created and selected by any suitable means. Preferred is combinatorial rearrangement of nucleic acid segments, for example in phage display libraries. Thus, the invention provides a chimeric protein domain according to any foregoing aspect of the invention, fused to the coat protein of a filamentous bacteriophage, said bacteriophage encapsidating a nucleic acid encoding the protein domain.

As used herein, the term “combinatorial library” refers to a collection of different polypeptides or nucleic acid sequences encoding such polypeptides, wherein each member of the collection comprises sequence segments from at least a first and a second source. One or both of such sources comprises a library of related, but different polypeptides or polynucleotides encoding them. The fusion of polypeptides (or polynucleotides encoding them) from the first source with polypeptides (or polynucleotides encoding them) from the second source results in the generation of a combinatorial library.

As used herein, the term “partner coding sequence,” used in the context of a combinatorial library, refers to a nucleic acid sequence from one source that is fused to a nucleic acid sequence from a second source to generate a fusion coding sequence comprised by a member of the combinatorial library.

Moreover, both configurations of the invention provide a nucleic acid encoding a protein domain according to the invention as defined above.

In a further aspect, the present invention relates to the de novo synthesis of recombinant folded proteins for use in therapeutic applications, including vaccination. In the context of this aspect of the invention only, the term “sequence segment” includes, in addition to the definition set forth above, an amino acid sequence which, in its parent environment, may comprise a single and complete protein structural element. The present invention, in the context of therapeutic applications and especially vaccines, therefore encompasses the juxtaposition of discrete and single elements of structure found in naturally-occurring or synthetic proteins, as well as the juxtaposition of blocks of more than one structural element or with the creation of novel structural elements by the juxtaposition of sequences which, in isolation or in their parent environments, do not possess a discrete and complete structure.

For the avoidance of doubt, any statement of invention or claim set forth herein, when referring to vaccines or therapeutic polypeptides, or polypeptides intended for therapeutic use, preferably encompasses the foregoing definition of “sequence segment” and thus relates to the combinatorial juxtaposition of polypeptide sequences consisting of partial, entire and/or multiple protein structural elements to form folded polypeptides. Advantageously, the fragments are derived from repertoires, as herein defined.

In a further aspect of both configurations of the invention, the amino acid sequences of any chimeric proteins can contain sequences designed to display epitopes for the vaccination against the parent protein of said amino acid sequences. For example, a chosen polypeptide segment from the coat protein of a virus, against which a vaccine is to be made, can be incorporated as a constitutive partner in a combinatorial library of amino acid sequences generated through the shuffling with one or more segments from another genetic source. Resulting chimeric proteins will then comprise the segment of the viral coat protein in a variety of structural environments. By screening or selection, for example using antibodies from antisera raised against the virus, it is possible to identify those folded chimeric proteins for which the viral sequence is displayed in a similar three-dimensional configuration to the viral protein. Such stably folded proteins among these chimeric constructs can be used for vaccination and elicit an immune response against the chimeric protein which includes the viral amino acid segment. Vaccination with such a protein results in immunisation against the virus. One advantage compared to vaccination with the viral coat protein is that it is thereby possible to focus the immune response against one defined epitope of the virus, such as a neutralisation epitope.

It is also possible to vaccinate against defined epitopes of human proteins by the same strategy by combining a segment from a human protein with that from another source. The segment of non-human source will provide T-cell epitopes that will lead to an immune response against the human epitope. By way of example, it is possible to raise a blocking (IgG) antibody response against the Fc portion of IgE that binds to the mast cell receptor (FcεRI). Such response is valuable therapeutically, for example, in blocking asthma. This is achieved by construction of a chimeric protein as follows. First, segments derived from IgE Fc are incorporated into chimeric proteins by combination with a repertoire of non-human segments; second, the chimeric polypeptide domains are screened for those that are properly folded, e.g., by binding to anti-IgE antibodies or to the mast cell receptor FcεRI (nucleotide sequences for FcεRI available in GenBank, e.g., XM054211, XM054212, and XM054210 (β polypeptides), XM048832, and XM002168 (α polypeptides), and XM042451 (γ polypeptide); the FcεRI polypeptides can be expressed on cells or cell membranes by one skilled in the art); third, the chimeric proteins with binding activities are administered to a patient. The IgE segments can be derived by random fragmentation of the IgE gene (nucleotide sequence available at GenBank Accession No. L00022), or by using a segment already known to interact with the mast cell receptor. For immunisation it can be necessary to build in more potent T-cell epitopes into the non-human part, which can be achieved by making mutations in the non-human segment.

Preferably, therefore, the chimeric protein according to the invention comprises an epitope of a parent amino acid sequence. Advantageously, the epitope is a structural or conformational epitope.

As used herein, the term “immune response” refers to the generation or activation of activities and molecules that participate in the elimination or neutralization of an immunogenic molecule. The activities and molecules that participate include, for example, production of cytokines, activation or proliferation of T cells, B cell activation, and antibody production. An immune response to an immunogenic molecule is specific to that molecule, which means that the response is directed to that molecule to the substantial exclusion of other, non-antigen, molecules. The term “substantial exclusion” means that an antibody raised in a humoral response binds the immunogenic molecule according to the invention with a K_{d} of 1×10⁻⁵ M or lower, preferably 10⁻⁶ M or lower, and more preferably 10⁻⁷ M, 1×10⁻⁵ M, or even 10⁻⁹ M or lower. For a cellular response immune response, e.g., activation of cytotoxic T cells, “substantial exclusion” means that the response kills only cells expressing an epitope of the antigen used to raise the immune response.

According to the invention, an immune response specific to an immunogenic molecule can be measured in vitro or in vivo by methods described herein below, including cytokine measurement, increased phagocytosis, activation of cytotoxic T cells, and T cell proliferation. An increase of at least 10%, or 20% or 30%, 40% or 50% or more, up to 2 fold or 3 fold or 5 fold or more in a measure of an immune response by one or more of these methods is an indication of an immune response.

For example, if a cell proliferation assay is used, an increase in cell number (e.g., at least 10%, 20%, 30%, 40% or 50% or more, up to 2 fold or 3 fold or 5 fold or more) in the presence of an immunogenic molecule, compared to the cell number in the absence of the immunogenic molecule, is indicative of an immune response specific to the immunogenic molecule. If, for example, a phagocytosis assay is used, an increase in phagocytosis (e.g., at least 10%, 20%, 30%, 40% or 50% or more, up to 2 fold or 3 fold or 5 fold or more) in the presence of an immunogenic molecule, compared to the cell number in the absence of the immunogenic molecule, is indicative of an immune response specific to the immunogenic molecule. If a cytotoxic T cell assay is used, an increase in cytotoxic activity of the T cells (e.g., at least 10%, 20%, 30%, 40% or 50% or more, up to 2 fold or 3 fold or 5 fold or more) in the presence of an immunogenic molecule, compared to the cytotoxic activity in the absence of the immunogenic molecule, is indicative of an immune response specific to the immunogenic molecule. If the production of cytokine or antibody is measured, an increase in cytokine or antibody production (e.g., at least 10%, 20%, 30%, 40% or 50% or more, up to 2 fold or 3 fold or 5 fold or more) in the presence of an immunogenic molecule, compared to the production of cytokine or antibody in the absence of the immunogenic molecule, is indicative of a modulation of an immune response specific to the immunogenic molecule.

As used herein, the term “epitope” refers to that portion of a polypeptide against which an immune response is raised (e.g., that portion recognised and bound by an antibody). The term “linear epitope” refers to an epitope made up of a sequence of contiguous amino acids. The terms “structural epitope” and “conformational epitope” refer to an epitope that, due to the tertiary structure of the polypeptide comprising the epitope, is made up of non-contiguous regions of the polypeptide. In a “conformational epitope,” one or more of the amino acids that participate in binding the antibody is/are not contiguous with the remaining amino acids which participate in binding, and the stable folding of the polypeptide brings the non-contiguous amino acids into juxtaposition such that they are recognised as an epitope.

Epitopes comprised in the chimeric proteins according to the invention, in a preferred embodiment, cross react with antibodies raised against a parent amino acid sequence, or, advantageously, the folded parent protein.

As used herein, the term “cross react” refers to the binding of an antibody to more than one polypeptide target. An antibody raised against a given first polypeptide is said to “cross react” with a second polypeptide if the antibody binds (i.e., physically associates with) the second polypeptide. In this situation, the second polypeptide is also said to “cross react” with the antibody. The binding of a cross reacting second polypeptide to the antibody preferably has the same or greater binding affinity relative to the affinity of the first (i.e., the K_{d} for the interaction of the second polypeptide for the antibody is equal to or less than that for the first polypeptide). However, as used herein, a second polypeptide can be said to “cross react” with an antibody if the K_{d} for binding is less than or equal or up to 100 fold higher than the K_{d} for the first polypeptide.

In a further aspect of both configurations of the invention the segments can be derived entirely from human proteins. It is expected that these proteins will be less immunogenic in humans than foreign proteins as the sequences of the protein will be almost entirely human. Although such novel human proteins will be expected to differ in three-dimensional structure from existing human proteins (and therefore to comprise novel B-cell epitopes), they will comprise T-cell epitopes derived from other human proteins (with the exception of the sequence flanking the join between segments). Such proteins, that are not immunogenic, or only weakly so, would be very suitable for therapeutic purposes or to avoid sensitisation in humans (for example enzymes in washing powders).

It is unlikely that in every respect the chimeric protein will mimic the three-dimensional surface of the original protein in the region of target segment. This can be desirable in that it can allow the protein to adopt a conformation that has altered binding activities. For example, such proteins can be valuable as improved enzyme inhibitors.

Moreover the invention in either configuration provides for the creation of small domains that mimic part of the surface of a larger protein. One advantage of small domains is that it can more readily permit the three-dimensional structure to be solved by X-ray crystallography or NMR, and also at higher resolution. In turn this can facilitate the design of non-protein drugs based on the structure.

Further the invention in either configuration allows for the fusion of individual sequence segments juxtaposed in the chimeric protein to additional, stably folded and complete protein domains. The function of additional domains can be to provide a means for selecting the chimeric protein domains (see methods below). They can also serve to complement the chimeric protein domain to perform a specific function, for example binding, immunogenicity or catalysis.

In the second configuration of the invention, the presence of at least two regions of the same sequence or similar (homologous) sequence in the chimeric protein can permit the development of chimeric proteins that bind to ligand at each of the two sites. This can be an advantage by giving improved “avidity” of binding where both heads can engage dimeric ligand (or other multimers), and also in providing two binding sites with different affinities, covering a larger dynamic range in binding to a ligand.

A further aspect of the first configuration of the invention relates to a method for selecting a protein domain according to the invention as defined above. Accordingly, the invention provides a method for preparing a protein domain according to the first aspect of the invention, comprising the steps of:

(a) providing a first library of nucleic acids, said library comprising coding sequences encoding sequence segments derived from one or more amino acid sequences, said coding sequences not being selected or designed such as to solely encode a single and complete protein structural element or to encode a complete protein domain;

(b) providing a second library of nucleic acids, said library comprising partner coding sequences encoding sequence segments derived from one or more amino acid sequences, said partner coding sequence not being selected or designed such as to solely encode a single and complete protein structural element or to encode a complete protein domain;

(c) combining the coding sequences to form a combinatorial library of nucleic acids, said nucleic acids comprising contiguous coding sequences encoding sequence fragments derived from the first and second libraries;

(d) transcribing and/or translating the contiguous coding sequences to produce the encoded protein domains;

(e) selecting the chimeric protein domains which are able to adopt a folded structure or to fulfill a specific function.

As used herein, the term “fulfill a specific function” means that a chimeric folded protein domain according to the invention performs the function for which it was selected. For example, a chimeric folded protein domain that is selected for the ability to bind a particular ligand “fulfills the specific function” of the protein domain if it binds to that ligand. Specific function can be any function that can be screened for, including, for example, ligand binding, recognition by an antibody (a subset of ligand binding), or catalysis of a given chemical reaction.

Libraries according to the invention can be constructed such that sequences homologous to the partner coding sequence are excluded. For example, the libraries can be based on an artificial combination of solved structures, which means that the presence or absence of sequences homologous to the partner coding sequence can be controlled. However, if genomic libraries are used, it is possible that sequences homologous to the partner sequence can be present. In a preferred aspect, therefore, the method according to the invention further includes the steps of:

(f) analysing the sequence of the selected chimeric protein domains to identify the origins of the sequence segments; and

(g) comparing the sequences of each of the parent amino acid sequences to identify whether the sequences of the parent amino acid sequences are non-homologous.

Similarly, it is possible to construct libraries comprising sequence segments derived from defined protein folds. However, if it is required to determine whether the isolated protein domain according to the invention is composed of sequence segments derived from parental domains having the same fold, the method according to the invention advantageously includes the step of:

(h) comparing the structures of each of the parent domains to identify whether they have same polypeptide folds in whole or in part.

In a further preferred aspect, the first configuration of the invention relates to the combination of a library of sequence segments with a unique partner coding sequence derived from a protein. The partner sequence is in this aspect provided as a unique sequence. Accordingly, steps (b) and (c) in the method according to the first configuration of the invention as set forth above can be modified such that:

(b) providing a partner coding sequence encoding a sequence segment derived from one protein, said partner coding sequence not being selected or designed such as to solely encode a single and complete protein structural element or to encode a complete protein domain;

(c) combining the library and partner coding sequences to form a combinatorial library of nucleic acids, said nucleic acids comprising contiguous coding sequences encoding sequence fragments derived from the first library and the partner coding sequence.

A further aspect of the second configuration of the invention relates to a method for selecting a protein domain, in which the individual sequence segments comprise common sequences.

Accordingly, the invention provides a method for preparing a protein domain according to the first aspect of the invention, comprising the steps of:

(a) providing a first library of nucleic acids, said library comprising coding sequences encoding sequence segments derived from one or more amino acid sequences, said coding sequences not being selected or designed to encode a complete protein domain;

(b) providing a second library of nucleic acids, said library comprising coding sequences encoding sequence segments derived from one or more amino acid sequences, said partner coding sequence not being selected or designed such as to solely encode a single and complete protein structural element or to encode a complete protein domain;

(c) combining the coding sequences to form a combinatorial library of nucleic acids, said nucleic acids comprising contiguous coding sequences encoding sequence fragments derived from the first and second libraries;

(d) transcribing and/or translating the contiguous coding sequences to produce the encoded protein domains;

(e) selecting the chimeric protein domains, which are able to adopt a folded structure or to fulfill a specific function; and optionally:

(f) analysing the sequence of the selected chimeric protein domains to identify the origins of the sequence segments; and

(g) comparing the sequences to identify whether they comprise common sequences.

Similarly, a further aspect of the second configuration of the invention relates to a method for selecting a protein domain, in which the individual sequence segments comprise common regions from parent proteins with a common fold. However, if it is required to determine whether the isolated protein domain according to the invention is composed of sequence segments derived from parental domains having the same fold, the method according to the invention advantageously does not require step (g) above, but includes in its place the steps of:

(g) comparing the structures of the parent amino acid sequences to identify whether the parent proteins have a common fold; and

(h) identifying whether the segments comprise a common region of the common fold.

In a further preferred aspect, the second configuration of invention also relates to the combination of a library of sequence segments with a unique partner coding sequence derived from a protein. The partner sequence is in this aspect provided as a unique sequence. Accordingly, steps (b) and (c) in the method according to the second configuration of the invention as set forth above can be modified such that:

(b) providing a partner coding sequence encoding a sequence segment derived from one protein, said partner coding sequence not being selected or designed such as to solely encode a single and complete protein structural element or to encode a complete protein domain;

(c) combining the library and partner coding sequences to form a combinatorial library of nucleic acids, said nucleic acids comprising contiguous coding sequences encoding sequence fragments derived from the first library and the partner coding sequence.

Preferably, in the methods according to the both configurations of the invention the domains which are able to adopt a folded structure are selected by one or several methods selected from the group consisting of in vivo proteolysis, in vitro proteolysis, binding ability, functional activity and expression.

As used herein, the term “in vitro proteolysis” refers to a process in which folded chimeric protein domains are selected from among a mixture comprising unfolded and folded chimeric protein domains. In this process, chimeric protein domains are treated with one or more proteases in vitro, and chimeric proteins that are not proteolytically cleaved are isolated.

As used herein, the term “in vivo proteolysis” refers to an alternative selection process for the identification of folded chimeric protein domains, wherein the proteolysis occurs in or on a cell. In this alternative process, the chimeric protein domains are expressed in or on a cell (e.g., as a phage coat protein fusion on the external surface of a phage-infected cell), and one or more proteases, either expressed in the cell or added to the culture medium, is used to select for chimeric polypeptides that are resistant to proteolysis.

As used herein, the term “binding ability” refers to the capacity of a folded chimeric polypeptide according to the invention to physically associate with a binding partner. “Binding ability” can be used to select chimeric folded polypeptide domains according to the invention by, for example, coating a desired binding partner (e.g., an antibody, a polypeptide or polypeptide domain, a nucleic acid, a hapten, etc.) onto a surface (e.g., a culture dish), placing a solution containing a plurality of chimeric folded polypeptide domains (e.g., expressed on the surface of a cell or phage) in contact with the surface, washing off unbound polypeptides and detecting or recovering bound, chimeric folded polypeptides according to the invention.

As used herein, the term “functional activity” refers to an enzymatic function possessed by a chimeric folded polypeptide domain according to the invention. By enzymatic function is meant catalysis of a reaction whereby substrate is converted to product with a K_{M} of at least 0.1 M, and preferably 10⁻³ M, 10⁻⁴ M, 10⁻⁵ M, 10⁻⁶ M, or even 10⁻⁷ M or lower. Those candidate chimeric polypeptide domains that have the given enzymatic function are selected as chimeric folded polypeptide domains over those that do not have the function.

As used herein, the term “expression,” when used relative to selection of a folded chimeric polypeptide domain according to the invention, means expression of candidate polypeptide domains in a cell or on the surface of a cell or bacteriophage. In this context, “expression” is used to select folded dimeric protein domains by screening phage or cell populations expressing the candidate polypeptide domains for physical association with a desired ligand or for a functional activity. Those cells or phage, which express or display chimeric protein domains that physically associate with the ligand or that express the desired functional activity are selected as chimeric folded polypeptide domains over chimeric polypeptide domains that do not fold to provide that binding or functional activity.

In a further aspect, an amino acid sequence of any chimeric proteins produced through combinatorial shuffling according to both configurations of the invention can be mutated or altered after the original juxtaposition of the parent amino acid sequences. Such changes can be introduced by any of the following methods:

(a) designing and introducing specific or random mutations at predefined positions within the gene of the chimeric protein;

(b) deleting nucleotides within the gene of the chimeric protein so as to delete amino acid residues;

(c) inserting nucleotides within the gene of the chimeric protein so as to insert amino acid residues

(d) appending nucleotides to the gene of the chimeric protein so as to append amino acid residues;

(e) randomly introducing mutations in all or part of the gene encoding the chimeric protein through recombinant DNA technology;

(f) randomly introducing mutations in the gene of the chimeric protein through propagation in mutator cells;

(g) introducing derivatives of natural amino acid during chemical synthesis;

(h) chemically derivatising amino acid groups after synthesis;

(i) multimerising the chimeric proteins through concatenation of two or more copies of the gene in a single open reading frame;

(l) multimerising the chimeric proteins through covalent linkage of two or more copies of the chimeric protein domain after translation;

(k) multimerising the chimeric proteins through fusion to a multimeric partner.

Any said changes can improve the stability or the function of the chimeric protein. For example, said changes can be aimed to meet predicted structural requirements within the combined segments advantageous for the formation of specific polypeptide folds or to introduce specific amino acid sequences to fulfill a desired function. An example of such improvements is given in Example 14 in the Experimental section.

As used herein, the term “altered” when used in reference to an amino acid sequence means that at least one amino acid in that amino acid sequence has been changed relative to a reference sequence. Alterations include random or targeted substitutions, deletions or additions of amino acid sequence, fusion of one amino acid sequence with another amino acid sequence, and multimerization of the amino acid sequence with itself or with another multimeric polypeptide.

As used herein, the term “mutation” refers to an insertion, deletion or substitution in a nucleotide sequence. Mutations can be point mutations, which alter a single nucleotide or amino acid, or they can alter larger tracts of nucleotide or amino acid sequence. Mutations can be “site directed” or “targeted,” meaning that a mutation is intentionally introduced at a limited number of sites, often only one site. Alternatively, mutations can be “random,” meaning that all of a given polynucleotide sequence is subject to mutation. The term “random mutagenesis” is also applied to mutagenic strategies that, while targeted to a particular region of a polynucleotide sequence, do not replace one or more specific amino acids with other specific amino acids, but rather replace them with any amino acid.

As used herein, the term “propagation through mutator cells” refers to the process of mutagenesis in which a polynucleotide sequence to be mutated is transfected into a cell strain that has a high rate of spontaneous mutagenesis, propagated for a limited time in those cells and re-isolated. Mutator cells are cells defective in at least one aspect of DNA repair, and which have at least 10 times the mutation frequency of a wild-type cell that is not defective in DNA repair, and frequently 100 times or 1000 times or greater mutation frequency relative to a wild-type cell.

The invention moreover encompasses further optimisation of the regions of N- and C-termini of recombined amino acid segments. Both their joining and end regions as part of a chimeric protein are conceivably not optimised as far as stability and/or function of the chimeric protein are concerned. Natural proteins, which can have been created through a recombinatorial event, are subsequently optimised through (point) mutational events and Darwinian selection. This process can be mimicked in vitro for chimeric protein as defined herein, for example using the above listed methods (including mutation, deletion and/or addition of amino acid residues).

As used herein, the term “increase stability” means that a given alteration to a chimeric folded protein domain according to the invention increases the free energy of folding of the domain by at least 10% relative to the domain prior to the alteration. Alternatively, “increase stability” means that a given alteration increases the amount of a sample of the altered protein that remains functional (e.g., for binding a known ligand) after protease digestion as described herein by at least 10%.

As used herein, the term “increase function” means that a given alteration to a chimeric folded protein domain according to the invention increases the binding affinity or catalytic function of the chimeric folded protein domain relative to the domain prior to the alteration. Binding affinity is considered “increased” if it is at least 10% greater after the alteration. Catalytic or enzymatic function is considered “increased” if the rate of substrate conversion to product is increased by at least 10%.

Chimeric proteins containing such improvements can be identified by one or more methods used for the selection and screening of the original combinatorial library. It can further be advantageous to produce any selected chimeric protein domains in a multimerised form, for example to increase stability through interdomain interaction or improve binding to a ligand through avidity effects.

Vaccines are frequently derived from a pathogenic agent, which has been rendered non-infectious prior to inoculation. Such vaccines have been often successful, but can carry inherent risks such as the possibility of remaining traces of toxicity or a reversion to virulence. To circumvent such problems, recombinant vaccines have been used, representing only a non-toxic but antigenic portion of the virulent substance or organism.

For a recombinant vaccine to be efficient in raising specific antibodies in a vaccinated organism, it must present both B cell and T cell epitopes. When the protection against a pathogen requires the fast activation of the immune response not only after encounter with the vaccine but also after encounter with the pathogen, the same B cell and T cell epitopes must be present on both vaccine and pathogen.

T cell epitopes are small fragments of a polypeptide chain derived from an antigen, which are created through proteolytic processing in the lymphocytes of an infected organism and are displayed on their surface bound by the MHC (Major Histocompatability Complex) molecules. The MHC-displayed fragments are recognised by different types of T cells, which activate macrophages (to destroy a pathogen) or B cells (to make pathogen- or antigen-specific antibodies).

The rapid production of large amounts of antigen specific antibodies depends on the antigen induced activation (i.e. the fast proliferation) of ‘primed’ B cells or memory cells. Activation of memory cells depends on antigen binding (i.e. presence of specific B cell epitopes on a pathogen) and interaction with helper T cells, which have previously encountered the antigen-derived, T cell epitope forming peptides displayed on the MHC of the B cell. Secreted antibodies bind then to the pathogen and initiate various other defence mechanisms depending on the isotype of their Fc portion. Both, pathogen-specific memory B cells and T helper cells require a previous encounter with their specific B and T cell epitope as presented by the antigen (i.e. vaccine or pathogen). Therefore a protective vaccine must usually comprise T and B cell epitopes that it shares with the pathogen.

In structural terms B cell epitopes can be divided into two groups. Continuous or linear epitopes are represented by a continuous polypeptide fragments of an antigen (i.e. vaccine or pathogen) and usually do not form a unique three dimensional structure (i.e. they are highly flexible). While some antibodies recognise linear B cell epitopes, many antibodies of an immune response recognise discontinuous or conformational B cell epitopes. Conformational epitopes are formed by the three dimensional structure of an antigen and comprise regions of a polypeptide chain, which are close together in space but not necessarily so in the primary amino acid sequence. These regions may even be part of altogether different polypeptides forming a single B cell epitope for example in a multi-protein complex.

The distinction between linear (or unfolded) and conformational epitopes is often difficult, as linear non-folded peptides (forming linear epitopes) may be able to adopt a conformation which is identical to or very similar to that within the folded antigen, only when they are bound to antibody but not in the absence of antibody. Therefore, for the purpose of this patent, conformational epitopes are defined as amino acid sequences which are stably folded (i.e. they show a co-operative folding behaviour and have a free energy of unfolding of for example at least 1.6 kcal/mol) in the absence of antibody ligand or other structure inducing agents, like for example the helix-inducing agent trifuoroethanol (U.S. Pat. No. 6,174,528; Cooper et al. 1997).

T cell epitopes can often be inferred from the amino acid sequence of an antigen and predicted proteosomal cleavages (Kuttler et al. 2000; and references therein). Molecules representing both T cell and linear B cell epitopes can be readily produced in the form of synthetic peptides or through the fusion of peptides to larger macromolecules. In contrast the design of conformational B cell epitopes, which are identical (or highly similar) in vaccine and pathogen, is more difficult.

A significant part of a natural antibody response is directed against conformational epitopes and it is therefore advantageous to use vaccines that display conformational epitopes shared with the pathogen. An antibody response against conformational epitopes is usually preferable, as it is directed against the active (folded) form of the antigen rather than denatured or proteolysed variants, which are only displaying linear epitopes.

Simple ways to design a vaccine presenting conformational epitopes include the use of a single, non-toxic polypeptide antigen from a virus coat, the engineering of a single, non-toxic domain from a multi-domain protein pathogen (Liljeqvist & Stahl 1999; and references therein) or a non-active mutant of a pathogen (for example EPO₃₂₂₅₃₃B). Such vaccines will often present conformational B cell epitopes of the pathogen. However, there are situations when this type of vaccine is not appropriate, amongst them: the B cell epitope, against which an immune response is desired, may not be naturally immunogenic; the molecule may present predominant B cell epitopes, which are not accessible on the native pathogen; or the molecule contains toxic characteristics due to other epitopes.

The present invention therefore concerns combinatorial protein domains for use in vaccination against at least one of the parent proteins, which can direct the immune response to specific and preferably conformational epitopes of an antigen (here parent protein). The present invention relates in this context to the use of de novo synthesised folded chimaeric protein domains, which comprise two or more sequence segments from parent amino acid sequences that are non-homologous, as vaccines. Such chimaeric proteins may share only B cell epitopes with the target antigen, leading to the presence specific antibodies in the vaccinated host but not a cell-mediated immune response against the target itself. Such antibodies may for example be therapeutically useful by blocking receptor sites (see above). Chimaeric proteins may also comprise both B cell and T cell epitopes from a parent protein leading to a cell-mediated immune response against it. The folded nature of the chimaeric protein domains allows the presence of conformational epitopes, while the restriction to structural elements of subdomain size can guide the immune response to specific epitopes. The possibility to duplicate structural elements (and hence conformational epitopes) within chimaeric proteins presents further means to optimise the efficiency of such chimaeric proteins as vaccines.

As far as the presence of T cell epitopes in a chimaeric protein is concerned, these can be included by design as part of one of the sequence segments forming the chimaera, when the T cell epitopes are known. Alternatively, they can be derived from predicted proteosomal cleavage sites or through prior vaccination tests in animal systems with linear peptides from the parent protein to be vaccinated against.

The selection of chimaeric proteins, which are folded and share specific B cell epitopes with at least one of its parent proteins, can be based on their ability to escape proteolytic attack (as a result of their folded nature) and/or their ability to bind antibodies raised against at least one of the parent proteins.

For example, a repertoire of chimaeric protein domains displayed on filamentous phage can be selected through exposure to proteases for those members which form stably folded domains. Selection for folded chimaeras will provide some discrimination against those presenting purely linear epitopes and in favour of those presenting conformational epitopes, as most amino acid sequence segments in folded chimaeric proteins will be locked into a stable three-dimensional conformation, which prevents these from adapting to the structural constraints of for example the antibody combining site for a linear epitope. In the pool of chimaeric proteins selected this way, those chimaeric proteins that share B cell epitopes with a parent protein can be detected by assaying for the binding of phage to immobilised unpurified antiserum (from the immunisation of a test animal with the parent protein), to affinity-purified polyclonal antibodies specific for the parent protein (using folded parent protein as an affinity-ligand) or to one or several monoclonal antibodies (each specific for a single and preferably conformational epitope on the parent protein). Alternatively, phage-displayed chimaeric proteins resulting from selection for folding can be enriched for those with antibody-binding function by further selection through panning of phage on unpurified antiserum (from the immunisation of a test animal with the parent protein), through panning on affinity-purified polyclonal antibodies specific for the parent protein (using folded parent protein as an affinity-ligand) or through panning on one or several monoclonal antibodies (each specific for a single and preferably conformational epitope on the parent protein).

The selection of chimeric protein domains, which share specific B cell epitopes with at least one of its parent proteins, can also based on initial panning on immobilised antibody (in the form of antiserum, affinity-purified polyclonal antibodies or one or several monoclonal antibodies specific for preferably conformational epitopes). However, especially binding to unpurified antiserum does not discriminate against chimaeric proteins that display only linear epitopes but no conformational epitopes, which are shared with the parent protein. Therefore selection of chimaeric proteins by antibody binding alone may result in the selection of many chimaeric polypeptides (but not always folded chimaeric domains), which display unstructured (i.e. highly flexible) linear epitopes from the parent protein. These may even be more abundant in a naive combinatorial library than stably folded chimaeric protein domains. It may therefore be advantageous to screen or select chimaeric polypeptides with a desired antibody binding activity further. This can include assays or selections for stability (e.g. proteolytic stability; co-operative folding/unfolding in temperature or urea denaturation; reduced binding to 1-anilinonaphthalene-8-sulfonate (Jones et al. 1994)) or function (e.g. binding to different source of antibody or ligand; enzymatic function).

When more than one monoclonal antibody is used for selection or screening, these may be used in combination, or advantageously in separate or sequential steps. Their sequential or separate application may be particularly advantageous, as it reduces the possibility of false positives (e.g. due to artefacts in the assay or the incidental ability of a flexible region within a folded chimaeric protein domain to adapt to the antibody combining site for a conformational epitope).

Finally repertoires of chimaeric proteins can be enriched for members, which are folded and share specific B cell epitopes with at least one of its parent proteins, based on a double selection for both stability and antibody binding (see above). The exact procedure of double selection can be modified by altering the number and the sequential order of selection steps but advantageously includes selection for stability and binding in each single round.

### BRIEF DESCRIPTION OF THE FIGURES

FIG. 1. Proteolysis of selected phages and chimeric proteins. (a) ELISA for barstar binding of phages 1c2 (squares), 1b11 (circles), 1 g6 (diamonds) and csp/2 (triangles) before and after trypsin/thermolysin treatment at different temperatures. (b) SDS-PAGE of proteins His-1c2, His-1b11 and His-1g6 before and after treatment with trypsin, thermolysin and chymotrypsin at 25° C.

FIG. 2. Circular dichroism and thermodenaturation of chimeric proteins. (a) Circular dichroism spectra of His-1c2 (upper trace) and His-2f3 (lower trace) at 20° C. (b) Ellipticity of His-1c2 (at 205 nm; upper trace) and His-2f3 (at 223 nm; lower trace) at different temperatures.

FIG. 3. Nuclear magnetic resonance analysis of chimeric proteins. 1D-¹H-NMR spectra of His-2f3 recorded (a) at 25° C. in H₂O and (b) after incubation for 24 hours at 25° C. in D₂O. 1D-¹H-NMR spectra of His-1c2 recorded at 30° C. (c) in H₂O and (d) after incubation for 24 hours at 25° C. in D₂O. 2D-¹H-NOESY spectrum of His-1c2 recorded at 30° C. (e) in H₂O.

FIG. 4. Antisera binding to CspA. Biotinylated CspA was bound to immobilised antisera from a rabbit, taken at different stages before after immunisation with CspA and detected with Streptavidin conjugated HRP. Antisera were immobilised on a Protein A coated ELISA plate.

### DETAILED DESCRIPTION OF THE INVENTION

The present invention relates to chimeric, folded protein domains. In the context of the present invention, “folded” means that the protein domains concerned are capable of adopting, or have adopted, a stable tertiary structure. Stability in this context can be defined as the conformational stability of the protein, which is the difference in free energy between the folded and unfolded conformations under physiological conditions; the higher this value, the greater the energy required to unfold the protein, and thus the greater the stability of the folded structure. A quantitative measure of this conformational stability of proteins, the Gibbs free energy of folding, can be determined from reversible thermodynamics. Proteins undergo order-disorder transitions, which are detectable in differential scanning calorimetry (DSC) profiles of specific heat vs. temperature.

According to the invention, the free energy of folding possessed by a chimeric folded protein domain according to the invention is 1.6 kcal/mol or higher; advantageously, it is 3 kcal/mol or higher; and most preferably it is 5 kcal/mol or higher.

Folded proteins which form stable structures are known to be resistant to proteolysis. Thus, the invention provides for the selection of folded protein domains in accordance with the present invention using protease enzymes, which cleave and preferably eliminate unstable or unfolded domains. “Folded” can therefore be alternatively defined in terms of “resistance to proteolysis” as defined herein, under assay conditions.

Sequence segments according to the invention are segments of natural protein sequence, which occurs in naturally-occurring proteins, or artificial segments of sequence modelled on the sequence or structure of naturally-occurring proteins. The sequence segments are at least 10 amino acids in length, generally between 10 and 100 amino acids or longer, preferably between 15 and 50 amino acids in length; and advantageously between 20 and 45 amino acids in length. Where nucleic acids are concerned, they are the necessary length to encode an amino acid sequence segment as defined herein.

Sequence segments according to the invention are derived from parental protein domains which are not homologous.

The term “parent amino acid sequences” (or “parental amino acid sequences”) refers to any amino acid sequences encoded by open reading frames within DNA sequences, which form the source of the cloned DNA segments as part of the combinatorial libraries described and claimed herein. Said reading frames can be part of the original reading frame of genes, of shifted reading frames or of the reverse strand of genes. They can also form part of intragenic regions, which are not known to encode a protein. Originating genes can be natural or synthetic.

As used herein, the term “open reading frame” refers to a nucleic acid sequence encoding at least 10 amino acids. An open reading frame is often defined in the art by the presence of a start codon (AUG) and a downstream stop codon. However, as used herein, the open reading frame need not begin with a start codon or end with a downstream stop-codon, as long as it encodes at least 10 amino acids. An open reading frame as used herein can occur in any reading frame on either strand of a nucleic acid molecule (i.e., there are three reading frames on a single strand of nucleic acid and six on a double-stranded molecule, any of which can be an “open” reading frame if it encodes at least 10 amino acids and no stop codon).

As outlined in the introduction, the term “homology” between two or more proteins or proteins domains can refer to a similarity or identity of both their amino acid sequences and their structural fold. For the present purposes, the term homology shall solely refer to the degree of identity between two parent amino acid sequences.

Homologous amino acid sequences have at least 35% or greater sequence identity (e.g., at least 40% identity, 50% identity, 60% identity, 70% identity, or at least 80% identity, such as at least 90% identity, or even at least 95% identity, for instance at least 97% identity). Homologous nucleic acid sequences are nucleic acid sequences which encode homologous polypeptides, as defined. Actual nucleic acid sequence homology/identity values can be determined using the “Align” program of Myers & Miller 1988, (“Optimal Alignments in Linear Space”) and available at NCBI. Alternatively or additionally, the term “homology”, for instance, with respect to a nucleotide or amino acid sequence, can indicate a quantitative measure of homology between two sequences. The percent sequence homology can be calculated as (N_{ref}−N_{dif})*100/N_{ref}, wherein N_{dif} is the total number of non-identical residues in the two sequences when aligned and wherein N_{ref} is the number of residues in one of the sequences. Hence, the DNA sequence AGTCAGTC will have a sequence similarity of 75% with the sequence AATCAATC (N_{ref}=8; N_{dif}=2). Alternatively or additionally, “homology” with respect to sequences can refer to the number of positions with identical nucleotides or amino acids divided by the number of nucleotides or I amino acids in the shorter of the two sequences wherein alignment of the two sequences can be determined in accordance with the Wilbur and Lipman algorithm (Wilbur & Lipman 1983), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to Uracil (U) in the RNA sequence.

RNA sequences within the scope of the invention can be derived from DNA sequences, by thymidine (T) in the DNA sequence being considered equal to Uracil (U) in RNA sequences.

Additionally or alternatively, amino acid sequence similarity or identity or homology can be determined using the BlastP program (Altschul et al. 1997) and available at NCBI. The following references (each incorporated herein by reference) provide algorithms for comparing the relative identity or homology of amino acid residues of two proteins, and additionally or alternatively with respect to the foregoing, the teachings in these references can be used for determining percent homology or identity: Needleman & Wunsch (1970); Smith & Waterman (1981); Smith et al. (1983); Feng & Dolittle (1987); Higgins & Sharp (1989); Thompson et al. (1994); and Devereux et al. (1984).

The invention contemplates the recombination of sequence segments which are derived from parental proteins with similar folds. In this context, “similar” is not equivalent to “homologous”. Indeed, similar folds have been shown to arise independently during evolution. Such folds are similar but not homologous.

As used herein, the term “polypeptide fold” refers to the tertiary structure of a polypeptide segment or a polypeptide domain, in the context of a parent polypeptide. The term “parent domains with the same polypeptide fold” means that a first polypeptide domain from one parent polypeptide has the same arrangement of secondary structural elements, and the same tertiary structure arrangement of those secondary structural elements, as a second polypeptide domain from another parent polypeptide. For example, if a first parent polypeptide domain has an α helix followed by a β sheet and assumes a given stable tertiary structure in the parent polypeptide, and a second parent polypeptide domain has an α helix followed by a β sheet and assumes the same tertiary structure arrangement of those elements as the first parent polypeptide domain, the parent domains are “domains with the same polypeptide fold.” The different types of polypeptide folds which have been observed in proteins are classified and described in Murzin et al. (1995).

Conversely, the term “parent domains with different polypeptide domains” means that a first polypeptide domain from one parent polypeptide has a different arrangement of secondary structural elements, and a different tertiary structure arrangement of those elements, than a second polypeptide domain from another parent polypeptide. For example, if a first parent polypeptide domain has an a helix followed by a β sheet, and a second parent polypeptide domain has a β sheet followed by a 3₁₀ helix, the parent polypeptide domains are “domains with different polypeptide folds.”

A “protein structural element” is an amino acid sequence which can be recognised as a structural element of a protein domain. Preferably, the structural element is selected from the group consisting of an α-helix, a β-strand, a β-barrel, a parallel or antiparallel β-sheet, other helical structures (such as the 3₁₀ helix and the pi helix), and sequences representing tight turns or loops. Advantageously, the structural element is an α-helix or a β-strand, sheet or barrel.

In a preferred embodiment, the folded protein domains according to the present invention are constructed from sequence segments which do not comprise only a single structural element; rather, they comprise less than a single structural element, or more than a single structural element or parts thereof.

In accordance with the present invention, the sequence segments used are not designed or selected to comprise only such single elements; in other words, they can comprise more than a single structural element, or less than a single structural element. This can be achieved through the use of substantially random sequence segments in constructing a library according to the invention. For example, sonicated genomic or cDNA or segments produced by random PCR of DNA can be used. Advantageously, the DNA fragments are between 100 and 500 nucleotides in length.

The sequence segments used in accordance with the present invention are unable to fold significantly in isolation; that is, they do not contain sufficient structural information to form a folded protein domain as the term “folded” is used herein unless they are combined with another sequence segment in accordance with the present invention. The inability to fold significantly can be measured by susceptibility to protease digestion, for example under the conditions given in the examples below, or by measurement of the free energy of folding.

Proteolysis can be carried out using protease enzymes. Suitable proteases include trypsin (cleaves at Lys, Arg), chymotrypsin (Phe, Trp, Tyr, Leu), thermolysin (small aliphatic residues), subtilisin (small aliphatic residues), Glu-C (Glu), Factor Xa (Ile/Leu-Glu-Gly-Arg), Arg-C (Arg) and thrombin. Advantageously, since the combination of random polypeptide sequence segments cannot be guaranteed to generate a precise cleavage site for a particular protease, a broad-spectrum protease or a mixture of proteases capable of cleaving at a variety of sites is used. Trypsin, chymotrypsin and thermolysin are examples of proteases useful in the present invention.

The ability of a protein domain to fold is also associated with its function. Accordingly, the invention provides for the selection of folded protein domains by functional assays.

In the case of immunoglobulins or other polypeptides capable of binding, such assays can be performed for binding activity according to established protocols; however, where binding is only transitory, the selection can be performed on the basis of function alone. Suitable methodology is set forth, for example, in International patent applications PCT/GB00/00030 and PCT/GB98/01889, both of which are incorporated herein by reference. Such techniques are useful for the selection of novel or improved enzymes produced by combinatorial rearrangement according to the present invention.

The invention also provides for screening for activity after selection according to protease resistance. This allows protein domains which have been selected according to their ability to fold to be screened for any desired activity. Since the repertoire sizes are more limited, as a result of the selection by proteolysis, the screening step can be conducted more easily (for example, in a multiwell plate).

According to one aspect of the invention, parent amino acid sequences include naturally occurring proteins and engineered proteins. Sequences for the expression of a great number of naturally occurring proteins (i.e., proteins having amino acid sequences found in nature) and/or the genes that encode them are available in databases such as GenBank and SwissProt. Those skilled in the art can readily express such naturally-occurring proteins and other, altered or engineered proteins using methods widely known in the art.

In another aspect of the invention, parent amino acid sequences include sequences of proteins with a known binding activity. Proteins with known binding activities include, for example, antibodies (preferably monoclonal antibodies), growth factor and cytokine receptors, DNA binding proteins, RNA binding proteins, and T cell receptors. Nucleotide and/or amino acid sequences for many proteins in this category are available in databases such as GenBank and SwissProt. Representative examples are as follows. GenBank Accession Nos. X91132 and X91128 provide mRNA sequences encoding antiphospholipid monoclonal antibody light and heavy chain V regions, respectively. GenBank Accession No. XM044655 provides the cDNA sequence of the human EFG receptor. GenBank Accession No. NM000206 provides the cDNA sequence for the human IL-2 receptor gamma. GenBank Accession No. NM021975 provides the cDNA sequence for the human DNA binding protein RelA. GenBank Accession No. K00788 provides the nucleotide sequence of human U1A RNA binding protein. GenBank Accession No. NM001698 provides the sequence of the human AU RNA binding protein/enoyl-Coenzyme A hydratase. GenBank Accession No. U40407 provides the complete cDNA sequence of a human/mouse chimeric T cell receptor alpha.

In another aspect of the invention, parent amino acid sequences include sequences of proteins with a known binding activity for an organic compound or for a carbohydrate. Examples of proteins in these categories are as follows. Genbank Accession No. AF050489 provides the complete cDNA sequence for the Microgadus tomcod aromatic hydrocarbon receptor (AhR), and numerous partial cDNA clones for the human AhR are also available. GenBank Accession No. 031813 provides the complete sequence of a vector encoding the carbohydrate-binding maltose binding protein. GenBank Accession No. NM022304 provides the sequence of the human histamine receptor H2.

In another aspect of the invention, parent amino acid sequences include those from proteins with a known binding activity for a polypeptide or peptide. In addition to the examples given above (antibodies, growth factor and cytokine receptors, etc.) numerous sequences encoding proteins with peptide or polypeptide binding activity are known and available in the databases. As examples, GenBank Accession No. XM047394 provides the sequence of the human CORO2A actin binding protein coronin; GenBank Accession No. XM046536 provides the sequence of the human xenotropic and polytropic retrovirus receptor; GenBank Accession No. NM005252 provides the sequence of the human c-Fos proto-oncogene protein, which has specific binding activity for c-Jun and for DNA.

In another aspect of the invention, parent amino acid sequences include those from proteins with a known binding activity for a hapten. Anti-hapten monoclonal antibodies are known in the art. Additionally, sequences for hapten-specific antibody domains are available in the public databases. Non-limiting examples include, GenBank Accession Nos. AJ278084 and AJ278083, AJ278082 and AJ27081, and AJ278081 and AJ278079, which encode murine immunoglobulin light and heavy chains, respectively, of different monoclonal antibodies specific for the hapten n(α)-(5′-phosphopyridoxyl)-L-lysine.

In another aspect of the invention, parent amino acid sequences include those from proteins with a known binding activity for an inorganic compound. Numerous sequences are known in the art and are available in public databases for polypeptides that bind various inorganic compounds. Non-limiting examples include sequences for metallothionein (GenBank Accession Nos. BC008408 (human, type 1H) and BC007034 (human, type 2A)), ferritin (GenBank Accession Nos. BC004245 (human, light chain) and BC001399 (human, heavy chain)), transferrin (GenBank Accession No. AH010951, human), calcium binding protein 2 (GenBank Accession No. AF170811, human), and selenium binding protein (GenBank Accession No. U29091, human).

In another aspect of the invention, parent amino acid sequences include those from proteins with a known binding activity for a steroid. Numerous steroid binding polypeptides are known in the art, particularly the steroid hormone receptors. Sequences for steroid binding polypeptides are available in the public databases. Non-limiting examples include sequences for estrogen receptor ((GenBank Accession Nos. AF258451, AF258450 and AF258449 (alternately spliced versions of human estrogen receptor α), AF051427 (human estrogen receptor β)), and progesterone receptor ((GenBank Accession No. NM000926, human).

In another aspect of the invention, parent amino acid sequences include those from proteins with a known enzyme activity. A great multitude of polypeptide sequences are available in public databases for proteins with known enzyme activity. Non-limiting examples of types of enzymes for which sequence information is available include hydrolases, nucleases, proteases, synthases, isomerases, polymerases, kinases, phosphatases, oxidoreductases and ATPases.

Antigens useful according to the invention:

In another aspect of the invention, sequence segments derived from an antigenic polypeptide are used to generate an immunogenic polypeptide that is folded in a manner similar to the way the sequence segment is folded in the context of the parent antigenic polypeptide. For this aspect, there are many antigenic polypeptides that can be used to advantage. Antigens derived from pathogenic bacteria, viruses and fungi are useful for raising a prophylactic immune response, e.g., as a vaccine. Further, tumor antigens or antigens involved in autoimmune disease can be useful.

For example, tumor antigens can be used to generate immunogenic polypeptides according to the invention, and the immunogenic polypeptides can be used to stimulate an anti-tumor immune response. An advantage of this approach is that tumor antigen epitopes (both linear and conformational), can be generated in the context of fused highly immunogenic sequences (e.g., bacterial cell surface or viral coat proteins) that effectively act as adjuvants to stimulate an anti-tumor response.

As another example, antigens involved in autoimmune disease can be used to generate immunogenic polypeptides according to the invention. These immunogenic polypeptides may then be able to saturate the binding sites of a patient's antibodies against the self antigen, which is involved in the autoimmune disease. Such immunogenic polypeptides may be presented in a multimeric form to create an avidity effect and thereby render them preferred ligands for the auto-antibodies compared to the self antigens.

Examples of antigens useful as vaccine compositions or therapeutic vaccine compositions according to the invention follow.

Tumor Antigens

A listing of human tumor antigens recognized by T cells is provided in a review article by Renkvist et al., 2001, Cancer Immunol. Immunother. 50:3-15, incorporated herein by reference. Examples of human tumor antigens and their GenBank Accession Nos. include the following: Prostate specific antigen (PSA; associated with prostatic carcinoma; see Osterling et al., 1991, J. Urol. 145: 907-923; GenBank Accession Nos. available for numerous human alleles, e.g., XM031764, XM031766, XM031767, XM031768, XM031769, XM031770, XM0317685); Human prostate cancer antigen KLK3 (KLK3; GenBank Accession No. AF394907); Epithelial membrane antigen (associated with multiple epithelial carcinoma types; see Pinkus et al., 1986, Am. J. Clin. Pathol. 85: 269-277; GenBank Accession No. M864683 (mouse));Cytokeratin 19 fragment 21-1 (CYFRA 21-1; see Lai et al., 1999, Jpn. J. Clin. Oncol. 29: 421; GenBank Accession No. AB045973, partial sequence); Ep-CAM (pan-carcinoma marker; see Chaubal et al., 1999, Anticancer Res. 19: 2237-2242; GenBank Accession No. NM002354); Carcinoma antigen GA733-2 (pan-carcinoma antigen; see Szala et al., 1990, Proc. Natl. Acad. Sci. U.S.A. 87: 3542-3546; GenBank Accession No. P16422); Alphafetoprotein (AFP, associated with hepatocellular carcinomas, male germ cell carcinomas; see Ghebranious et al., 1995, Mol. Reprod. Dev. 42: 1-6; GenBank Accession No. XM003498); placental alkaline phosphatase (GenBank Accession Nos.: X66946 and X66947 (both human); see also Deonarain et al., 1997, Protein Eng. 10: 89-98; Travers & Bodmer, 1984, Int. J. Cancer 33: 633-641) and Erb-B2 (associated with breast cancer; see Pandha et al., 1999, J. Clin. Oncol. 17:2180; GenBank Accession No. XM049823).

Other human tumor markers include, but are not limited to sialyl-Lewis X (adenocarcinoma, Wittig et al., 1996, Int. J. Cancer 67: 80-85), CA-125 and CA-19 (“CA” refers to “cancer antigen” in the common nomenclature; associated with gastrointestinal, hepatic, and gynecological tumors; Pitkanen et al., 1994, Pediatr. Res. 35: 205-208), TAG-72 (colorectal tumors; Gaudagni et al., 1996, Anticancer Res. 16: 2141-2148), epithelial glycoprotein 2 (pan-carcinoma expression; Roovers et al., 1998, Br. J. Cancer. 78: 1407-1416), pancreatic oncofetal antigen (Kithier et al., 1992, Tumor Biol. 13: 343-351), 5T4 (gastric carcinoma; Starzynska et al., 1998, Eur. J. Gastroenterol. Hepatol. 10: 479-484,; alphafetoprotein receptor (multiple tumor types, particularly mammary tumors; Moro et al., 1993, Tumour Biol. 14: 11-130), M2A (germ cell neoplasia; Marks et al., 1999, Brit. J. Cancer 80: 569-578), OV-632 (associated with ovarian cancer), CA-50, CA-242, CA-15-3, Rb, endocrine granule constituent (EGC), β-subunit of human chorionic gonadotropin, Thomsen-Friedenreich antigen (TF-α. TF-β), MAGE-1, CT-9 (CT refers to “cancer/testis”), CT-10, MAGE (melanoma-associated antigen)-B5, -B6, -C2, -C3 and -D, placental alkaline phosphatase, sialyl-Lewis X and mutant β-catenin.

Viral Antigens:

A wide variety of viral antigens are known in the art and have sequences available in the public databases. The entire genome sequences are available in GenBank for a number of viruses. Open reading frames from these viral genomes can be used to generate immunogenic chimeric polypeptide domains according to the invention. Generally, however, for a given virus, specific viral polypeptides will be known that can serve as a source for sequence segments useful in generating an immunogenic chimeric polypeptide domain according to the invention. Examples include influenza virus hemagglutinin (e.g., GenBank Accession Nos. AF186269, AF186268, AF186267, and AF186266), rubella virus HA (GenBank Accession No. L16233), rotavirus (e.g., GenBank Accession Nos. AF401754, AF323719, and AF 323716), rhinovirus (a complete genome sequence is GenBank Accession No. NC001490; capsid sequences include AF152281 and AF152253), hepatitis A virus (a complete genome sequence is GenBank Accession No. AB020569; capsid sequences include GenBank Accession Nos AJ254535 and AF394945), measles virus (a complete genome sequence is GenBank Accession No. NC001498; hemagglutinin sequences include AF107017 and AF107016), respiratory syncytial virus (a complete genome sequence is GenBank Accession No. NCO01803; glycoprotein sequences include AJ410856 and AJ410852), hantavirus (glycoprotein—GenBank Accession No. AF366569; nucleocapsid—GenBank Accession No. AF366568), herpes virus 1 (glycoprotein D—GenBank Accession No. AF293614), rabies virus (glycoprotein sequences include GenBank Accession Nos. AF177096, AF177095 and AF177094), lassa virus (glycoprotein/nucleocapsid sequences—GenBank Accession No. AF246121) and HIV (numerous env glycoprotein sequences available, including GenBank Accession Nos. AF403530, AF403529 and AF403528).

Bacterial Antigens:

Numerous bacterial antigens are known in the art, and sequences are available for them in the public databases. In a number of instances, entire genome sequences are available, providing a rich source of antigen sequences. In others, specific genomic regions and specific gene sequences are known. Non-limiting examples include antigens from Aeromonas hydrophila (e.g., GenBank Accession No. AF276639, outer membrane protein; and GenBank Accession No. L77573, cytotoxic enterotoxin), Streptococcus pyogenes (e.g., GenBank Accession No. AY039661 and AY039660, M protein; GenBank Accession No. AB040536, fibronectin binding protein, and GenBank Accession No. L26150, pyrogenic exotoxin), Propionibacterium acnes (e.g., GenBank Accession No. U15927, hyaluronidase; and GenBank Accession No. X99255, lipase), Bacillus anthracis (GenBank Accession No. AJ304809, cereolysin; and AF367984, β-lactamase), Bacillus subtilis (GenBank Accession No. AB039913, flagellin; and GenBank Accession No. AJ222890, phytase), Bordetella pertussis (GenBank Accession No. AF348488, pertactin; and GenBank Accession No. AY032627, outer membrane heme receptor), Campylobacter jejuni (GenBank Accession No. AF369587, flagellin; and GenBank Accession No. AF023133, integral membrane protein B), Clostridium difficile (GenBank Accession Nos. X17194 and U25132, enterotoxin A; GenBank Accession No. AF095238, flagellin), Enterobacter aerogenes (GenBank Accession No. AF373860, OmpC-type protein; and GenBank Accession No. AF357597, β-lactamase), Klebsiella pneumoniae (GenBank Accession No. U52843, outer membrane protein 17; and GenBank Accession No. AJ344089, outer membrane protein 36), Proteus vulgaris (GenBank Accession No. D29982, β-lactamase; and GenBank Accession No. AF030017, outer membrane protein R), Salmonella typhi (GenBank Accession No. AF234268, outer protein D; GenBank Accession No. AF213333, outer protein B; and AJ313032, cytolysin), Shigella dysenteriae (GenBank Accession No. U64516, outer membrane heme receptor; GenBank Accession No. AF010147, virulence determinant; GenBank Accession No. D26166, flagellin), Helicobacter pylori (GenBank Accession No. AF291095, vacuolating toxin; and GenBank Accession No. AY034551, outer membrane protein A) and Staphylococcus aureus (GenBank Accession No. U48826, elastin binding protein; and GenBank Accession No. AF325855, feric hydroxamate receptor).

Fungal Antigens:

Numerous fungal antigens are known in the art, and sequences are available for them in the public databases. Non-limiting examples include antigens from Candida albicans (GenBank Accession Nos. AF229990, and AF068866, agglutinin-like proteins), Histoplasma capsulatum (GenBank Accession Nos. AF315588 and AF159366, chitinases), and Aspergillus niger (GenBank Accession No. AJ290451, alpha glucuronidase; and GenBank Accession No. AJ276331, pectate lyase).

Protozoal Antigens:

Examples of protozoal and other parasitic antigens include, but are not limited to, plasmodium falciparum antigens such as merozoite surface antigens, sporozoite surface antigens, circumsporozoite antigens, gametocyte/gamete surface antigens, blood-stage antigen pf 155/RESA and other plasmodial antigen components, toxoplasma antigens such as SAG-1, p30 and other toxoplasmal antigen components, schistosomae antigens such as glutathione-S-transferase, paramyosin, and other schistosomal antigen components, leishmania major and other leishmaniae antigens such as gp63, lipophosphoglycan and its associated protein and other leishmanial antigen components, and trypanosoma cruzi antigens such as the 75-77 kDa antigen, the 56 kDa antigen and other trypanosomal antigen components.

Libraries and Vectors:

The libraries of the present invention can be created by any suitable means in any form. As used herein, the term “library” refers to a mixture of heterogeneous polypeptides or nucleic acids. The library is composed of members, each of which has a unique polypeptide or nucleic acid sequence. To this extent, library is synonymous with repertoire. Sequence differences between library members are responsible for the diversity present in the library. The library can take the form of a simple mixture of polypeptides or nucleic acids, or can be in the form organisms or cells, for example bacteria, viruses, animal or plant cells and the like, transformed with a library of nucleic acids. Typically, each individual organism or cell contains only one member of the library. In certain applications, each individual organism or cell can contain two or more members of the library. Advantageously, the nucleic acids are incorporated into expression vectors, in order to allow expression of the polypeptides encoded by the nucleic acids. In a preferred aspect, therefore, a library can take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

A number of vector systems useful for library production and selection are known in the art. For example, bacteriophage lambda expression systems can be screened directly as bacteriophage plaques or as colonies of lysogens, both as previously described (Huse et al. (1989); Caton & Koprowski (1990); Mullinax et al. (1990); Persson et al. (1991) and are of use in the invention. While such expression systems can be used for screening up to 10⁶ different members of a library, they are not really suited to screening of larger numbers (greater than 10⁶ members). Other screening systems rely, for example, on direct chemical synthesis of library members. One early method involves the synthesis of peptides on a set of pins or rods, such as described in WO84/03564. A similar method involving peptide synthesis on beads, which forms a peptide library in which each bead is an individual library member, is described in U.S. Pat. No. 4,631,211 and a related method is described in WO92/00091. A significant improvement of the bead-based methods involves tagging each bead with a unique identifier tag, such as an oligonucleotide, so as to facilitate identification of the amino acid sequence of each library member. These improved bead-based methods are described in WO93/06121.

Another chemical synthesis method involves the synthesis of arrays of peptides (or peptidomimetics) on a surface in a manner that places each distinct library member (e.g., unique peptide sequence) at a discrete, predefined location in the array, or the spotting of pre-formed polypeptides on such an array. The identity of each library member is determined by its spatial location in the array. The locations in the array where binding interactions between a predetermined molecule (e.g., a receptor) and reactive library members occur is determined, thereby identifying the sequences of the reactive library members on the basis of spatial location. These methods are described in U.S. Pat. No. 5,143,854; WO90/15070 and WO92/10092; Fodor et al. (1991); and Dower & Fodor (1991).

Of particular use in the construction of libraries of the invention are selection display systems, which enable a nucleic acid to be linked to the polypeptide it expresses. As used herein, a selection display system is a system that permits the selection, by suitable display means, of the individual members of the library.

Any selection display system can be used in conjunction with a library according to the invention. Selection protocols for isolating desired members of large libraries are known in the art, as typified by phage display techniques. Such systems, in which diverse peptide sequences are displayed on the surface of filamentous bacteriophage (Scott & Smith (1990)), have proven useful for creating libraries of antibody fragments (and the nucleotide sequences that encode them) for the in vitro selection and amplification of specific antibody fragments that bind a target antigen. The nucleotide sequences encoding the VH and VL regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phagebodies). An advantage of phage-based display systems is that, because they are biological systems, selected library members can be amplified simply by growing the phage containing the selected library member in bacterial cells. Furthermore, since the nucleotide sequence that encodes the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (McCafferty et al. (1990); Kang et al. (1991); Clackson et al. (1991); Lowman et al. (1991); Burton et al. (1991); Hoogenboom et al. (1991); Chang et al. (1991); Breitling et al. (1991); Marks et al. (1991); Barbas et al. (1992); Hawkins & Winter (1992); Marks et al. (1992); Lemer et al. (1992), incorporated herein by reference).

Other systems for generating libraries of polypeptides or polynucleotides involve the use of cell-free enzymatic machinery for the in vitro synthesis of the library members. For example, in vitro translation can be used to synthesise polypeptides as a method for generating large libraries. These methods, which generally comprise stabilised polysome complexes, are described further in WO88/08453, WO90/05785, WO90/07003, WO91/02076, WO91/05058, and WO92/02536. Alternative display systems which are not phage-based, such as those disclosed in WO95/22625 and WO95/11922 (Affymax), use the polysomes to display polypeptides for selection. These and all the foregoing documents are incorporated herein by reference.

In order to produce libraries of sequence segments in accordance with the present invention, PCR amplification is advantageously employed. Where a defined partner sequence is used, PCR primers can be designed to anneal specifically with the partner sequence; for random libraries, general random PCR primers can be used. The resulting fragments are joined by restriction and ligation and cloned into suitable vectors. Although the ligation of two sequence segments is described below, the invention encompasses the ligation of three or more sequence segments, any of which can be the same or different, such as to mirror a multiple cross-over event.

Selection and Screening Strategies

Selection and screening of repertoires of chimaeric protein domains can be based on the distinction between folded and nonfolded entities. This can be achieved, as outlined in Examples 1 to 22 below, through the exposure of the chimaeric protein domains to proteases, which preferentially cleave peptide bonds within nonfolded polypeptides. Exposure to proteases is most easily controlled in vitro using purified preparations of the chimaeric proteins (isolated or when fused to other proteins or indeed phage particles) and defined conditions (i.e. buffer, temperature and proteases concentrations). The fusion of chimaeras to phage is particularly advantageous as it links the phenotype (the phage-surface displayed chimaeric protein) to its sequence (the genotype or DNA for the chimaeric protein which is encapsulated in the phage particle). However when using for example arrays of chimaeric protein domains in the form of chemically synthesised peptides for the screening of repertoires, the sequence information is given by the position of the chimaeric protein in the array. In that instance, no link to a genotype is required and phage-display has no advantages.

The distinction between folded and nonfolded members of a repertoire of chimaeric protein domains can also be achieved by other means than proteolytic resistance, none of which requires phage-display. Other properties, which can be used for such a distinction, include but are not limited to:

(1) Survival from proteolytic attack by constitutively expressed proteases in the cell producing the chimaera is likewise increased by the folded nature of a chimaera. As described in Example 22, cells expressing chimaeric proteins resistant to in vivo proteolysis can be detected through the remaining link of N- and C-terminal tags, which can be used for immobilisation on a solid support and detection respectively.

(2) Biophysical properties of a chimaeric protein, like fluorescence as in the Green Fluorescent Protein, depend on the folded state of a protein and therefore allow the differentiation of folded and nonfolded chimaeras. Thus cells expressing different chimaeras, of which some may exhibit fluorescent properties, can be selected using FACS (Fluorescence Activated Cell Sorting) based on the intensity of their fluorescence. Clones of cells selected in this way can be analysed for the DNA or protein sequence of the chimaera produced by them.

(3) Enzymatic properties of a chimaeric protein can be used to select or screen for folded chimaeras, as such functions usually depend on the coming together of different reactive groups in a defined tertiary structural context only present in a folded polypeptide. For example chimaeric proteins, which augment a metabolic process in a clone of a bacterial strain with a defect in this process, will give this clone a growth/survival advantage leading to its enrichment. The sequence of the chimaera can be determined directly from the purified protein or from its gene present in the enriched cells.

(4) Binding properties of a chimaeric protein can be used for its detection. For example chimaeric proteins secreted by bacterial colonies grown in arrays on nitrocellulose filters can be screened for binding to a ligand immobilised on a second nitrocellulose filter through detection of a C-terminal tag to the chimaeric protein. Position of the signal for the C-terminal tag in the array replicated on the second filter indicates the identity of the bacterial clone in the array, from which the sequence of the chimaera can be determined by sequencing the chimaeric gene present in the bacterial clone.

However, while the methods above (and likewise the experimental examples below) will lead to the selection or detection of particular chimaeric proteins with distinct properties or functions, isolated chimaeric proteins must be analysed for folding to verify the formation of their folded nature. This can be done as outlined in Example 7 or by any other means leading to the determination of their folding energy and/or their structure determination.

Assays for Determining an Immune Response According to the Invention

The efficacy of a molecule according to the invention in causing an immune response can be tested in a variety of ways known in the art (e.g., see Current Protocols in Immunology, 1995, Vols. 6 and 7). The assays described in detail below measure stimulation or suppression of cellular or humoral immune responses to an antigenic molecule of the invention. The antigens referred to in the following assays are representative. A number of antigens useful according to the invention are listed in Table IV. It will be apparent to one of skill in the art that an immune response to a selected antigen useful according to the invention may be measured using one or more of the following assays by adapting the assay to that antigen.

Measuring Cytokine Production

This protocol describes an immunonoenzymetric assay for cytokines using a heterogeneous, noncompetitive immunoassay reaction in which the cytokine is immobilized by a coating antibody bound to a microtiter plate. Unbound material is washed free, and detection is carried out using a different anti-cytokine antibody labeled with the hapten nitroiodophenyl (NIP). This is in turn detected by a horseradish peroxidase (HRPO) conjugate of an anti-NIP antibody, which is revealed with the chromogenic substrate ABTS. In this noncompetitive immunoassay, the immunoassay signal (A₄₀₅) increases as a direct function of the amount of cytokine present in the sample. Antibodies are prepared as described in Current Protocols in Immunology, 1995, 6.20.2-6.20.10.

Coat Assay Plate.

(1) Using a multichannel pipettor, transfer 100 ul of an appropriate dilution of coating antibody into all wells of the assay plate that are to be used. (2) Seal plates with microtiter plate sealer or Parafilm and incubate 2 hr at 37° C. Prepare samples and standards in preparation plate. (3) Dilute each sample (or aliquot of conditioned medium) to be assayed with an equal volume of immunoassay diluent. (4) Pipet less than or equal to 1 ml of each diluted sample to be assayed into the upper chamber of a separate Spin-X microfiltration device. Microcentifuge 5 min at 10,000 rpm and save the filtrates that collect in the lower chambers. (5) Add 65 μl of each diluted sample to the appropriate well of a preparation plate (i.e., a separate 96-well microtiter plate). (6) Thaw an aliquot of cytokine standard at room temperature and make sure that it is well mixed. Pipet 130 μl into the well of the preparation plate representing the highest concentration on the standard curve. Transfer 65 ul from this well into the next, then continue performing serial 1:1 dilutions in immunoassay diluent so that 65 ul of each concentration represented on the standard curve is placed in the appropriate well of the preparation plate. (7) Thaw an aliquot of calibrator at room temperature (if used). Dilute with an equal volume of immunoassay diluent, then pipet 65 ul of diluted calibrator into the appropriate well or wells of the preparation plate.

Incubate with Coating Antibody.

(8) Remove coated assay plate from incubator. Dip in 2-liter beaker filled with 1× wash buffer, then invert over sink and flick to remove liquid. Repeat two more times, then bang dry on paper towel. (9) Transfer 50 ul of solution from each well of preparation plate to corresponding well of the assay plate using a multichannel pipettor. (10) Seal plate with microtiter plate sealer or Parafilm and incubate 2 hr. at room temperature.

Incubate with Detecting Antibody.

(11) Dilute NIP-labeled detecting antibody specific to cytokine of interest to 1 ug/ml in detecting buffer. (12) Wash assay plate as in step 8. (13) Add 75 ul diluted detecting antibody from step 11 to all wells of assay plate, including unused outer walls. (14) Reseal plate with microtiter plate sealer or Parafilm and incubate 1 hr. at room temperature.

Incubate with HRPO-Conjugated Anti-NIP Antibody.

(15) Dilute HRPO-conjugated anti-NIP Mab 1:3000 in detecting buffer. (16) Wash assay plate as in step 8. (17) Add 75 ul of diluted HRPO-labeled anti-NIP antibody from step 15 to all wells of assay plate. (18) Reseal plate with microtiter plate sealer or Parafilm and incubate 1 hr. at room temperature.

Incubate with Chromogenic Substrate.

(19) Wash assay plate as in step 8. (20) Add 100 ul ABTS substrate working solutions to all wells of assay plate. Cover plate and incubate at room temperature until color development reaches desired level (generally until A₄₀₅ for wells containing the highest concentration of standard is between 1.5 and 2). This protocol usually produces an assay that can be read after 30 to 60 min.

Read Plate and Analyze Data.

(21) Using microtiter plate reader with computer interface, measure absorbance in all wells at 405 nm in single-wavelength mode or at 405 and 650 nm in dual-wavelength mode. (22) Fit standard data to a curve described by a first-degree (linear), second degree (quadratic), or four-parameter (nonlinear) mathematical function using curve-fitting software. (23) Interpolate absorbance data from unknown cytokine samples to fitted standard curve, and calculate cytokine concentrations.

Detection of Increased Phagocytosis

Phagocytosis is examined using monocytes that have been adhered at 37° for 30 min in RPMI without added FCS. Sheep erythrocytes are incubated with immunogenic molecule of the invention under conditions such that there are no more than 300 of such molecules, on average, are deposited on each erythrocyte. Fresh monocytes are isolated from the subject, and 5×10⁴-1×10⁵ of these cells suspended in 0.25-0.5 ml of RPMI medium with 1% BSA. This aliquot is placed in a tissue culture well and incubated for 30 min at 37° C. An excess of coated erythrocytes, suspended at 1.2×10⁸ cells/ml, is overlain on the monocytes, the plate is centrifuged for 5 min at 50 g, and incubated for 30 min at 37° C. Non-ingested material is removed in two hypotonic lysis steps using ice-cold lysing buffer before fixing and staining the adherent cells, and examining the cells under light microscopy. Phagocytosis is quantified by determining the percentage of 100 monocytes ingesting one or more target cells, and the total number of ingested E/100 monocyptes (PI) is recorded. Stimulation of phagocytosis according to the invention is indicated by a phagocytic index of equal to or greater than 40.

Cytotoxic T Cell Assay

T cells are incubated overnight at 37° C. with 5 μg/mL of an immunogenic molecule in 1 mL of complete medium. The next day, cells are washed once and labeled with 100 μCi of Na⁵¹Cr (Amersham) for 1 hr. Target cells then were washed 4 times with medium and incubated with different numbers of T-cells for 4 hrs in round-bottomed 96-well plates. Percent specific cytotoxicity was calculated as by the formula: 100[(ER−SR)/(MR−SR)], where ER (experimental ⁵¹Cr release)=cpm released into the supernatant in the presence of T-cells using 5×10³ target cells in triplicate samples; SR (spontaneous ⁵¹Cr release)=cpm in the absence of T cells determined from four replicate samples, and MR(maximal ⁵¹Cr release)=cpm in supernatant of target cells incubated with 0.5% Nonidet-P40(Sigma) determined from four replicate samples. SR is always <20% of MR.

Cell Proliferation Assays

The procedure outlined here measures incorporation of [³H]thymidine into DNA, which usually correlates well with cell growth as measured by changes in cell number. T cell proliferation which measures the proliferation of T cells specific for an immunogenic molecule of the invention can be performed as follows.

(1) Add 20 ul of 50 uCi/ml [³H]thymidine to each T cell culture (1.0 uCi) with or without the presence of an immunogenic molecule at a fixed time before terminating the culture (usually 6 or 18 hr). (2) Harvest cell cultures using an automated multiwell harvester that aspirates cells, lyses cells, and transfers DNA onto filter paper, while allowing unincorporated [³H]thymidine to wash out. Fill and aspirate each row of the microtiter plate ten times to ensure complete cell transfer and complete removal of unincorporated thymidine. Wash each filter strip with 100% ethanol to facilitate drying. Transfer to scintillation vials. For semiautomated harvester, transfer filter dots for each well into scintillation counting vials. For manual transfer, dry filters under lamp and transfer to scintillation vial with forceps. Add scintillation fluid to each vial. (3) Count samples in scintillation counter until standard deviation is less than 2%. Calculate mean cpm for background cultures and for each experimental condition. There should be less than 20% variation in replicate cultures.

Animal Models

An immunogenic molecule according to the invention can be administered by intramuscular, intravenous, or subcutaneous route. The in vivo immune response to an administered immunogenic molecule is tested (e.g., in animals) by any one of the assays described above and below, using immune cells or blood isolated from immunized animals.

Animals such as mouse, guinea pig and rabbit may be immunized with an immunogenic molecule emulsified in Freund's adjuvant or other adjuvant. After three injections (5 to 100 μg peptide per injection), IgG antibody responses may be tested by peptide-specific ELISAs and immunoblotting against the immunogenic molecule.

Serum is collected from vaccinated animals (e.g., mouse, rabbit or guinea pig) and control animals as follows: draw preimmunized blood sample, allow blood to clot, and separate serum from clot by centrifugation. Store serum at −20° C. to −70° C.

Animal models of disease are useful for determining the efficacy of treatment using immunogenic compositions according to the invention. For example, a guinea pig model for allergic asthma is described by Savoie et al., 1995, Am. J. Respir. Cell Biol. 13: 133-143. An animal model for multiple sclerosis is experimental autoimmune encephalomyelitis (EAE), which can be induced in a number of species, e.g., guinea pig (Suckling et al., 1984, Lab. Anim. 18: 36-39), Lewis rat (Feurer et al., 1985, J. Neuroimmunol. 10: 159-166), rabbits (Brenner et al., 1985, Isr. J. Med. Sci. 21: 945-949), and mice (Zamvil et al., 1985, Nature 317: 355-358).

There are numerous animal models known in the art for diabetes, including models for both insulin-dependent diabetes mellitus (IDDM) and non-insulin-dependent diabetes mellitus (NIDDM). Examples include the non-obese diabetic (NOD) mouse (e.g., Li et al., 1994, Proc. Natl. Acad. Sci. U.S.A. 91: 11128-11132), the BB/DP rat (Okwueze et al., 1994, Am. J. Physiol. 266: R572-R577), the Wistar fatty rat (Jiao et al., 1991, Int. J. Obesity 15: 487-495), and the Zucker diabetic fatty rat (Lee et al., 1994, Proc. Natl. Acad. Sci. U.S.A. 91” 10878-10882).

There are also animal models for prostate disease (Loweth et al., 1990, Vet. Pathol. 27: 347-353), atherosclerosis (numerous models, including those described by Chao et al., 1994, J. Lipid Res. 35: 71-83; Yoshida et al., 1990, Lab. Anim. Sci. 40: 486-489; and Hara et al., 1990, Jpn. J. Exp. Med. 60: 315-318), nephrotic syndrome (Ogura e tal., 1989, Lab. Anim. 23: 169-174), autoimmune thyroiditis (Dietrich et al., 1989, Lab. Anim. 23: 345-352), hyperuricemia/gout (Wu et al., 1994, Proc. Natl. Acad. Sci. U.S.A. 91: 742-746), gastritis (Engstrand et al., 1990, Infect. Immunity 58: 1763-1768), proteinurialkidney glomerular defect (Hyun et al., 1991, Lab. Anim. Sci. 41:442-446), food allergy (e.g., Ermel et al., 1997, Lab. Anim. Sci. 47: 40-49; Knippels et al., 1998, Clin. Exp. Allergy 28: 368-375; Adel-Patient et al., 2000, J. hmunol. Meth. 235: 21-32; Kitagawa et al., 1995, Am. J. Med. Sci. 310: 183-187; Panush et al., 1990, J. Rheumatol. 17: 285-290), rheumatoid disease (Mauri et al., 1997, J. Immunol. 159: 5032-5041; Saegusa et al., 1997, J. Vet. Med. Sci. 59: 897-903; Takeshita et al., 1997, Exp. Anim. 46: 165-169), osteoarthritis (Rothschild et al., 1997, Clin. Exp. Rheumatol. 15: 45-51; Matyas et al., 1995, Arthritis Rheum. 38: 420-425), lupus (Walker et al., 1983, Vet. Immunol. Immunopathol. 15: 97-104; Walker et al., 1978, J. Lab. Clin. Med. 92: 932-943), and Crohn's disease (Dieleman et al., 1997, Scand. J. Gastroenterol. Supp. 223: 99-104; Anthony et al., 1995, Int. J. Exp. Pathol. 76: 215-224; Osborne et al., 1993, Br. J. Surg. 80: 226-229).

These accepted animal model systems, or others known and accepted in the art to be representative of human disease, can be used to test the efficacy of therapeutic approaches using immunogenic polypeptides according to the invention. Generally, this is accomplished by administering the immunogenic polypeptide composition to an animal that has or can be induced to have the model disease corresponding to the human disease one aims to treat, and monitoring the disease status. The immunogenic response to the administered composition is then monitored using, for example, any of the assays described herein. Disease status is monitored according to criteria established for the particular disease or disease model, and treatment is considered effective if one or more symptoms or markers of disease are decreased by 10% or more relative to animals not treated or relative to the same animal before treatment.

As an example, the treatment of allergic asthma with an immunogenic polypeptide according to the invention can be evaluated in the following manner. Mice, e.g., specific pathogen-free BALB/c mice, are sensitized by intraperitoneal injections of 10 μg of ovalbumin on alternating days for a total of 7 injections. Four weeks later, animals are challenged with ovalbumin aerosol inhalation (nebulized 2 mg/ml ovalbumin solution). Airway hyperresponsiveness is measured using, as a gauge, “Penh,” the enhanced pause, which is a non-invasive system for evaluating the allergen-specific airway response in a murine model system of asthma (see Dohi et al., 1999, Lab. Invest. 79: 1559-1571, incorporated herein by reference). Additionally, serum anti-ovalbumin-specific IgE can be monitored, bronchoalveolar lavage can be performed to monitor eosinophilia following challenge, and tissue samples can be evaluated for cytokine expression (e.g., IL-4, IL-5 and IL-13, the increased expression of which correlate with asthma). For this example, the immunogenic polypeptide composition according to the invention carries a chimeric protein domain comprising a segment of the murine IgE constant region; the chimeric protein domain is selected for its ability to bind murine mast cell IgE receptor as target polypeptide. The immunogenic polypeptide composition is administered to the animal in a course of injections, and efficacy against asthma is determined by ovalbumin aerosol challenge of the animal. A decrease (i.e., at least 10%) in Penh after ovalbumin challenge, relative to the value of Penh in sensitized, challenged animals that did not receive the immunogenic polypeptide composition, is indicative of therapeutic efficacy of the treatment. Such a decrease is also indicative of an immune response to the immunogenic polypeptide composition according to the invention. Alternatively, or in addition, other indicators of asthma as described above can be monitored to similar effect.

Measuring In Vivo Immune Response

Each serum sample is analyzed by enzyme-linked immune adsorbent assay (ELISA). The immunogenic molecule used for vaccination in phosphate-buffered physiologic saline (PBS) is adsorbed onto the wells of a polystyrene plate at 4° C. for 18 hours (e.g., 50 mg/well). The subsequent steps were performed at room temperature. Following adsorption, each well is washed several times with PBS and 0.5% gelatin in PBS is added and allow to adsorb for one hour. The wells are then washed once with PBS and a 1:20 dilution, in PBS, of each test serum is added in duplicate wells and allow to react with the absorbed peptide for two hours. The wells are subsequently washed once with the 0.5% gelatin solution, twice with 0.02% Tween-20 detergent solution in PBS and three times with PBS.

A 1:250 dilution of a 1.0 mg/ml solution of goat anti-mouse IgG, goat anti-rabbit IgG or goat anti-guinea pig IgG antibodies, covalently linked to alkaline phosphatase, is then added to the appropriate wells and allow to react for two hours. The wells are then washed twice with 0.02% Tween-20 in PBS, twice with PBS, and three times with water. Each well received 0.1% p-Nitrophenyl phosphate in 10% diethanolamine, pH 9.8, containing 0.5 mM MgCl₂.6H₂O. The ensuing reaction was allowed to proceed at 37° C. for 30 minutes, at which time it was terminated by the addition of sodium hydroxide.

The greater the interaction of antibodies in the test serum with the peptide substrate, the greater is the amount of alkaline phosphatase bound onto the well. The phosphatase enzyme mediates the breakdown of p-nitrophenyl phosphate into a molecular substance which absorbs visible light at a wavelength of 405 nm. Hence, there exists a direct relationship between the absorbance of 405 nm light at the end of the ELISA reaction and the amount of peptide-bound antibody.

It is understood that other enzymes or molecules generating detectable signals can be used to replace alkaline phosphatase and other modifications may be employed for the above-described assay.

Generation of T Cell Lines Specific for an Immunogenic Molecule of the Invention

Peripheral blood mononuclear cells (PBMC) from immunized animals may be isolated from the heparinized blood by centrifugation through a Ficoll/Hypaque (Pharmacia LKB Biotech. Inc.) gradient. PBMCs (2.5×10⁶ cells/mL) in a 24 well plate (Gibco) are incubated with the immunogenic molecule used for immunization (5×10⁵ PFU/mL) in “complete medium” (RPMI 1640 medium (Sigma) containing 2 mM L-glutamine, 25 mM Hepes, 50 mM penicillin, 50 mM streptomycin and 5.times.10-5 M 2-mercaptoethanol) supplemented with 10% autologous plasma. After incubation at 37° C. for 7 days, the cells are washed 3 times with medium and resuspended at 1×10⁶ cells/mL in “complete medium” supplemented with 10% fetal calf serum (FCS) and 100 U/mL of human recombinant IL-2 (Cetus). After 7 day-incubation, an antigen-specific proliferation assay is performed as described above.

Vaccine Compositions

An immunogenic composition made according to the methods of the invention can advantageously be administered to raise an immune response to an epitope carried by that composition, e.g., as a vaccine. Vaccines of this type are useful not only to prevent infection by an organism comprising an epitope recognized by antibodies raised to the vaccine composition, but also therapeutically, for example in inducing a cytotoxic response to a cell involved in a pathology.

Preparation of vaccine compositions is well known to those skilled in the art. An immunogenic ” compound useful in a vaccine composition is conveniently in injectable form, for example in solution or suspension, but can be prepared in dry or solid form suitable for suspension in a liquid prior to administration. The immunogenic compound can also be emulsified in liposomes. In one aspect, immunogenic compounds of the invention will comprise non-human polypeptide sequences that act as adjuvants, the immunogenic compositions of the invention can be mixed with an adjuvant before administration to enhance the immune response.

Immunogenic compounds are most often combined with a “pharmaceutically acceptable carrier” for administration to an individual. A “pharmaceutically acceptable carrier” is one that does not cause an allergic reaction or other untoward effects in subjects to whom it is administered. Examples include water, saline, phosphate-buffered saline, dextrose, glycerol, ethanol or the like and combinations thereof. Tissue culture growth medium is expressly excluded from the term “pharmaceutically acceptable carrier.”

Dosage and Administration

Immunogenic compositions according to the invention can be administered parenterally by injection, e.g., subcutaneously or intramuscularly. Alternatively, formulations can be prepared for administration as suppositories, aerosols, or possibly even oral preparations. Oral formulations include normally-employed excipients, such as pharmaceutical grades of mannitol, lactose, starch, magnesium stearate, sodium saccharine, cellulose, magnesium carbonate and the like. Vaccine compositions take the form of solutions, suspensions, tablets, pills, capsules, sustained release formulations or powders and contain 10%-95% of active ingredient, preferably 25-70%.

Chimeric folded polypeptides of the invention can be formulated into the vaccine or treatment compositions as neutral or salt forms. Pharmaceutically acceptable salts include the acid addition salts (formed with free amino groups of the peptide) and those formed with inorganic acids such as, for example, hydrochloric or phosphoric acids, or with organic acids such as acetic, oxalic, tartaric, maleic, and the like. Salts formed with the free carboxyl groups can also be derived from inorganic bases such as, for example, sodium, potassium, ammonium, calcium, or ferric hydroides, and such organic bases as isopropylamine, trimethylamine, 2-ethylamino ethanol, histidine, procaine, and the like.

Immunogenic compositions are administered in a manner compatible with the dosage formulation, and in such amount as will be prophylactically and/or therapeutically effective. The quantity to be administered depends on the subject to be treated, including, e.g., capacity of the subject's immune system to synthesize antibodies, and the degree of protection or treatment desired. Suitable dosage ranges are of the order of several hundred micrograms active ingredient per vaccination with a preferred range from about 0.1 mg to 1000 mg, such as in the range from about 1 mg to 300 mg, and preferably in the range from about 10 mg to 50 mg. An exemplary unit dose suitable for vaccination is 0.5-5 microgram of antigen/kg. Suitable regimens for initial administration and booster shots are also variable but are typified by an initial administration followed by subsequent inoculations or other administrations. Precise amounts of active ingredient required to be administered depend on the judgment of the practitioner and may be peculiar to each subject. It will be apparent to those of skill in the art that the therapeutically effective amount of chimeric folded polypeptides of the invention will depend, inter alia, upon the administration schedule, the unit dose of antigen administered, whether the polypeptide is administered in combination with other therapeutic agents, the immune status and health of the recipient, and the therapeutic activity of the particular chimeric folded polypeptide.

The compositions can be given in a single dose, or preferably in a multiple dose schedule. A multiple dose schedule is one in which a primary course of vaccination can include 1-10 separate doses, followed by other doses given at subsequent time intervals required to maintain and or reinforce the immune response, for example, at 1-4 months for a second dose, and if needed, a subsequent dose(s) after several months. Periodic boosters at intervals of 1-5 years, usually 3 years, are desirable to maintain the desired levels of protective immunity.

The invention is further described, for the purpose of illustration, in the following Examples.

### EXAMPLES

### Example 1

Preparation of a Repertoire of Chimeric Proteins Comprising Two Sequence Segments

A repertoire of genes encoding chimeric proteins, which comprise the N-terminal 36 residues of the E. coli cold shock protein (CspA; nucleotide sequence at GenBank Accession No. M30139) and a C-terminal polypeptide sequence encoded by randomly created fragments of the E. coli genome, was prepared. CspA comprises 70 residues and forms a stable β-barrel (Schindelin et al. 1994). Its N-terminal 36 residues comprise the first three strands of its six stranded P-barrel and are unable to fold when expressed alone as they are degraded in the E. coli cytoplasm.

The gene fragment encoding the first 36 residues of CspA was complemented with fragmented DNA from the E. coli genome around 140 base pairs in size. DNA fragments were created by random PCR amplification using genomic E. Coli DNA as a template. Resulting chimeric genes were inserted between the coding regions for the infection protein p3 and an N-terminal tag, a stable but catalytically inactive mutant of the RNase bamase, as a single continuous gene on a phagemid vector for protein display on filamentous phage. Those skilled in the art can also express such chimeric polypeptides on the surface of non-filamentous phage, e.g., lambda.

In the resulting genomic library (1.0×10⁸ members) an opal (TGA) stop codon was incorporated at the 3′ end of the chimeric gene in 60% of clones with the remainder containing the Gly-encoding GGA codon in this position. The partial incorporation of the TGA codon at the 3′ end of the chimeric genes was achieved through the use of two different PCR primers (XTND and NOARG) in the PCR amplifications of the E. coli gene fragments. The transfer-RNA^{TrP} can decode TGA with an efficiency of up to 3% (Eggertsson & Soll 1988) leading to sufficient display of the bamase-chimera-p3 fusion on the phage but avoiding folding related, toxic effects. Phages displaying this repertoire were prepared using the helper phage KM13, which contains a modified fd gene 3 encoding a trypsin-sensitive p3 due to a modified sequence (Kristensen & Winter 1997), to reduce infectivity due to helper phage encoded p3 molecules.

### Example 2

Preparation of a Repertoire of Chimeric Proteins Comprising Two Sequence Segments with Common Sequences

In a second “plasmid-derived” library the N-terminal CspA gene fragment was complemented with DNA fragments of around 140 base pairs created by random PCR amplification using as the PCR template a 3.6 kb plasmid containing the wild type CspA gene. Resulting chimeric genes were again inserted as a fusion between the coding regions for the infection protein p3 and an N-terminal tag, a stable but catalytically inactive mutant of the RNase bamase, on a phagemid vector for protein display on filamentous phage.

In the plasmid-derived library (1.7×10⁸ members) an opal (TGA) stop codon was constitutively introduced at the 3′ end of the chimeric gene in all clones. Phages displaying this repertoire were prepared using the helper phage KM13, which contains a modified fd gene 3 encoding a trypsin-sensitive p3 due to a modified sequence (Kristensen & Winter 1997), to reduce infectivity due to helper phage encoded p3 molecules.

### Example 3

Proteolytic Selection of Combinatorial Libraries

To select stably folded chimeric proteins from the repertoires of bamase-chimaera-p3 fusions described in Examples 1 and 2, the phage-displayed libraries were selected for proteolytic stability in three rounds through treatment at 10° C. with the proteases trypsin (specific for peptide bonds containing Arg or Lys in the P₁ position) and thermolysin (specific for bonds containing a amino acid with an aliphatic side chain in the P₁, position) followed by capture on barstar, elution, infection and regrowth.

After the first round of selection, 2×10⁴ and 6×10² of 10¹⁰ proteolytically treated phages were eluted from a single barstar coated microtitre plate well in case of the plasmid-derived library and the genomic library, respectively. When protease treatment is omitted 5×10⁶ phages can be eluted indicating that the vast majority of unselected phages did not display a stably folded chimera protein fused between bamase and p3. The number of phages rescued after two and three rounds of selection increased to 2×10⁵ for the plasmid-derived library and to 2×10³ and 4×10⁴ for the genomic library.

Selected phages were grown up individually, bound to immobilised barstar, treated in situ with trypsin and thernolysin at 10° C. and resistance was measured through detection of bound (and therefore resistant) phage in ELISA. For the plasmid-derived library 27 of 64 phages (42%) retained 80% or more of their barstar binding activity after protease treatment. For the genomic library, after two rounds, 6 of 192 (3%) phages retained at least 80% of their barstar binding activity. After three rounds, 31 of 86 (36%) phages retained 80% or more of their barstar binding activity. Selection therefore clearly enriched phages displaying protease-resistant p3 fusions.

### Example 4

Sequence Analysis of Selected Chimeric Proteins

As an initial characterisation of the selected chimeric fusion proteins, the sequences of the selected clones from Example 3 were determined. The chimeric genes of all the 24 most stable phage clones selected from the plasmid-derived library had an open reading frame from the genes for bamase, through the one for chimeric protein and to the end of the p3 gene. They also contained no stop codons (in addition to the opal stop codon at the 3′ end). Twenty of these contained inserts originating from the CspA gene in the correct reading frame. These 20 comprised three different clones (A1 was found 12-times, D6 6-times, G4 twice). Phage A1 contains a deleted version (residues 1 to 52) of the CspA wild type gene, which must have been created through a deletion within a phagemid clone originally harbouring a larger insert (Table 1). Phage D6 contains in addition to the N-terminal half of CspA (residues 1 to 36 as part of the cloning vector) the core of CspA (residues 17 to 53) (Table 1). Phage G4 contains as an insert a partial duplication of the N-terminal half of CspA (residues 2 to 19). Thus from the plasmid-derived library, phages with p3-fusion chimeras, in which the N-terminal half of CspA was complemented with another fragment from CspA, were strongly enriched by proteolytic selection.

The sequences of 25 protease resistant phage clones selected from the genomic library revealed 11 different clones (2 clones were found five times, 1 clone four times, 3 clones twice). All inserts kept the reading frame from bamase into p3. They all contained the opal stop codon at their 3′ end but no additional stop codons. The inserts of all phages sequenced could be traced back to the E. coli genome showing an error-rate of about 1% presumably due to their generation by PCR. 64% of the sequenced phages contained inserts, whose reading frame was identical to that of the originating E. coli protein. This suggests an enrichment for DNA fragments in their natural reading frame, as from a random distribution based on three possible reading frames and two possible orientations of any DNA only 16% of inserts would be expected to retain the natural reading frame. However, the selection of clones which originated from open reading frames (ORFs) that do not correspond to the natural reading frame of the originating gene in 36% of the sequenced inserts indicates that these can also lead to the formation of stably folded chimaeras.

As outlined in Example 1, 60% of all clones in the unselected genomic library contained an opal (TGA) stop codon at the 3′ end of the chimeric gene while the remainder contained the Gly-encoding GGA codon in this position. However, only clones containing opal stop codons at this position were found after proteolytic selection from the genomic library. In the absence of a constitutive stop codon almost exclusively chimeric gene fusions leading to a frameshift between the bamase and p3 genes were selected (data not shown). These results show that the efficiency (up to 3% according to Eggertsson & Söll, 1988), with which transfer-RNA can decode TGA as a tryptophan, leads to sufficient display of the barnase-chimera-p3 fusion on the phage but appears to reduce folding related, toxic effects. The use of an opal stop codon in the genes encoding the displayed fusion proteins was therefore advantageous for selection in the presented examples.

### Example 5

Proteolytic Stability of Selected Chimaera-Phages in Solution

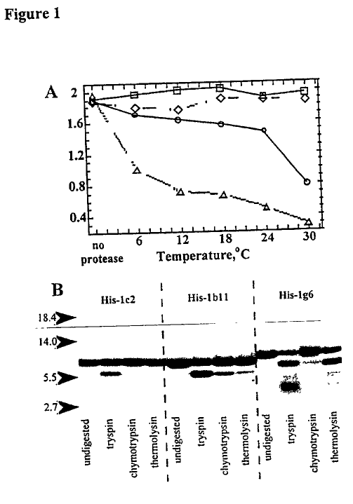

To show that the sequenced fusion proteins were not only proteolytically stable after immobilisation of the displaying phage on a barstar coated surface (as shown in Example 3) but also in solution, they were tested for proteolytic stability through exposure to trypsin and thernolysin in solution (prior to immobilisation) at different temperatures (FIG. 1a). Phages retaining the barnase tag (as a consequence of a proteolytically stable fusion protein) were captured on barstar and the percentage of retained barstar binding activity was quantitated by ELISA.

Among the phages from the plasmid-derived library two clones (A1 and D6) retained at least 80% of their binding activity after treatment at 20° C. From the genomic library 8 of the 11 clones (1C2, 1G6, 1A7, 2F3, 1B11, 2F1, 2H2, 3A12) retained at least 80% of their activity after trypsin/thermolysin treatment at 24° C. The remaining phages were less well protected from proteolytic attack in solution than when bound to the barstar coated surface (compare Example 3).

### Example 6

Soluble Expression of Selected Chimeric Proteins

To characterise the selected chimeric proteins outside the context of the bamase-p3 fusion protein, the genes of the ten most stable chimaeras of the selected clones in Example 5 were expressed without the fusion partners. For this, their genes were subcloned for cytoplasmic expression into a His-tag vector. Five of these proteins (His-a1, His-d6 from the plasmid-derived library; His-1c2, His-2B3 and His-1b11 from the genomic library) could be purified after expression directly from the soluble fraction of the cytoplasm via their His-tag. The remaining proteins formed inclusion bodies in the expressing cells. One of these, His-1 g6 containing an insert expressed in a reading frame different from that of its originating gene (Table II), was refolded via solubilisation in 8M urea. The remaining clones were not further studied.

### Example 7

Biophysical Characterisations of Chimeric Proteins

The first biochemical analysis of the purified chimeric proteins described in Example 6 concerned their multimerisation status. The chimeric proteins His-al, His-d6, His-lc2, His-2f3, His-1g6 formed only monomers according to their elution volume in gel filtration, while His-1b11 formed 30% monomers with the remainder forming dimers.

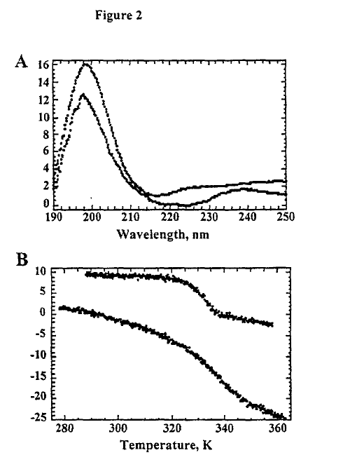

To analyse the type of secondary structure formed by these chimaeras, the purified proteins were studied by CD and NMR. The CD spectra (FIG. 2a) of the monomeric proteins and the monomeric fraction of His-1b11 were all characteristic of β-structure containing proteins with minima between 215 nm and 225 nm (Greenfield & Fasman 1969, Johnson 1990). All proteins exhibited cooperative folding characteristics with sigmoidal melting curves (FIG. 2b) and midpoints of unfolding transition between 46° C. and 62° C. (Table 1). The cooperative folding behaviour is a strong indication that each of the analysed chimaeras forms a domain with a single fold, in contrast to a mixture of folded or partially folded structures as in a molten globule.

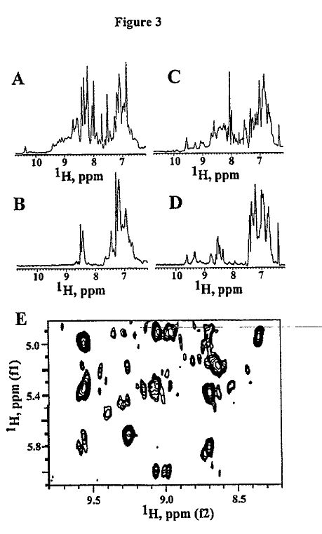

The NMR spectra of His-2f3 and His-1c2 further suggested the presence of well folded protein domains, as can be inferred from the chemical shift dispersion of many amide protons to values 1 downfield of 9 ppm (FIG. 3a, c) and of methyl group protons to values around 0 ppm in their NMR spectra (Wüthrich 1986). Finally, downfield chemical shifts of C^{α} protons to values between 5 and 6 ppm, as seen in the NMR spectrum of His-1c2 (FIG. 3e), are also frequently observed in β-sheet containing polypeptides like the immunoglobulin domains (Riechmann & Davies 1995).

To determine the thermodynamic stability of the selected chimaeras, the energy of unfolding (ΔG) of the six proteins was inferred from their thermodenaturation curves as measured by CD (FIG. 2b). The folding energies of His-a1, His-d6, His-1b11, His-2f3 and His-1 g6 are between 1.6 and 2.4 kcal/mol (Table I). These values are lower than those of typical natural proteins and similar to the so far most stable of the de novo designed β-structure proteins, betadoublet (2.5 kcal/mol; Quinn et al. 1994). However, the His-1c2 protein selected from the genomic library had a considerably higher folding energy of 5.3 kcal/mol, which falls within the normal range of natural proteins (5 to 15 kcal/mol; Pace 1990). His-1c2 is indeed 1.7 kcal/mol more stable than His-CspA.

The relative folding stabilities of His-2f3 and His-lc2 were confirmed through the rate of exchange of their amide protons in D₂O as observed in NMR experiments. For His-2f3 a 1D-¹H NMR spectrum recorded after incubation for 24 hours in D₂O buffer at 25° C. revealed the complete exchange of its amide protons (FIG. 3a, b). In contrast, amide exchange in His-1c2 was slow allowing the observation of many amide protons in a 1D-¹H NMR spectrum after 24 hours at 25° C. in D₂O (FIG. 3c,d). A group of amide signals between 8.7 and 10 ppm was even detectable three weeks later at about 40% of their original intensity.

### Example 8

Proteolytic Stability of Chimaeras as Soluble Proteins

Apart from the spectroscopic evidence for folding stability (see Example 7), stability was also confirmed by the exposure of the isolated chimeric proteins to proteases in solution. The stability data described in Example 7 of the soluble chimeric proteins from Example 6 largely correspond to the degree of their protection from proteolysis by trypsin, thermolysin (both used during the selection) and chymotrypsin (FIG. 1b). Tryptic degradation of the N-terminal His-tag through cleavage after Arg11 was observed for all six proteins. This arginine was introduced as part of the expression vector immediately C-terminal of the N-terminal His-tag. His-1 c2 (with a folding energy of 5.3 kcal/mol) is no further degraded by any of the proteases confirming its high conformational stability, but the other proteins are partially proteolysed within the main body of the polypeptides. This is consistent with a partial unfolding expected from a folding energy of about 2 kcal/mol. Thus although all the proteins are resistant to proteolysis (for example compared with the facile cleavage of the His-tag at Arg), the resistance varies between the proteins and upon the conditions.

### Example 9

Sequence Duplications in Selected Chimeric Proteins

As outlined in Example 4 above, in 20 of 24 sequenced chimeric proteins, which were selected from the plasmid-derived library, the N-terminal half of CspA was complemented with another fragment from CspA. Indeed, the chimeric proteins D6 and G4 both comprise a partial duplication of their N-terminal half. Phage D6 contains in addition to the N-terminal half of CspA (residues 1 to 36 as part of the cloning vector) the core of CspA (residues 17 to 53) (Table 1). Phage G4 contains as an insert a partial duplication of the N-terminal half of CspA (residues 2 to 19). This result indicates that (partial) duplication of amino acid segments can lead to the formation of stably folded protein domains.

### Example 10

Duplication of Homologous Elements in Stably Folded Chimeric Proteins

No direct structural information is available for the seven DNA fragments, which were found after selection of the genomic library (Example 1) and which were expressed in their natural reading frame. One however has a high level of sequence identity with a sequence neighbour of known three-dimensional structure (as identified by BLAST analysis of the E. coli genome). The insert of phage 1B11 spans residues 364 to 398 in the E. coli 30S ribosomal subunit protein S1 (gene identifier 1787140), of which residues 369 to 397 have a 52% identity with residues 11 to 39 of S1 RNA-binding domain from the E. coli polynucleotide phosphorylase. These comprise a stretch of four β-strands in the 3D structure of the S1 domain, which like CspA forms a β-barrel albeit with an inserted helix (Bycroft et al. 1997).

The two S1 domains (of the 30S ribosomal protein and of the phosphorylase) are according to their sequence similarity and identity homologous to CspA. The juxtaposition of the segments in the chimeric protein 1B11 represents therefore a juxtaposition of corresponding regions from homologous polypeptide domains (which also forming the same structural fold). This result indicates that a (partial) duplication of homologous amino acid segments can lead to the formation of stably folded protein domains.

### Example 11

Evidence for Complementation with Elements of Similar Structure from Proteomic Analysis in a Chimeric Protein

20 of the 24 most stable phage clones selected from the plasmid-derived library (Example 2) contained inserts originating from the CspA gene in the correct reading frame (see Example 4).

These 20 comprised three different clones (A1, D6, G4). A1 contains a deleted version (residues 1 to 52) of the CspA wild type gene, which must have been created through a deletion within a phagemid clone originally harbouring a larger insert (Table 1). Phage D6 contains in addition to the N-terminal half of CspA (residues 1 to 36 as part of the cloning vector) the core of CspA (residues 17 to 53) (Table 1). Phage G4 contains as an insert a partial duplication of the N-terminal half of CspA (residues 2 to 19). The complementing sequences in all three clones comprise regions of CspA, which in the CspA structure form β-strand regions. Thus sequences forming the same type of secondary structure are juxtaposed in the chimeric proteins A1, D6 and G4.

No direct structural information is available for the seven DNA fragments, which were found after selection of the genomic library (Example 1) and which were expressed in their natural reading frame. One however has a high level of sequence identity with a sequence neighbour of known three-dimensional structure (as identified by BLAST analysis of the E. coli genome). The insert of phage 1B11 spans residues 364 to 398 in the E. coli 30S ribosomal subunit protein S1 (gene identifier 1787140), of which residues 369 to 397 have a 52% identity with residues 11 to 39 of S1 RNA-binding domain from the E. coli polynucleotide phosphorylase. These comprise a stretch of four P-strands in the 3D structure of the S1 domain, which like CspA forms a β-barrel albeit with an inserted helix (Bycroft et al. 1997). Thus sequences forming the same type of secondary structure are juxtaposed in the chimeric protein 1B11.

Thus in case of the His-a1, His-d6 and His-1b11 proteins the juxtaposition of sequences, which form the same type of secondary structure, have led to the formation of stably folded chimeric protein. Overall, gene fragments selected from both libraries appear to be enriched for sequences forming primarily β-structure in their parent protein. Such sequences can be more frequently able to form a stable domain with another gene fragment, that originally encodes part of a ≢-barrel, than sequences of a helical origin.

### Example 12

Evidence for Complementation with Elements of Different Structure from Proteomic Analysis in Selected Chimeric Proteins

No direct structural information is available for the seven DNA fragments, which were found after selection of the genomic library (Example 1) and which were expressed in their natural reading frame. One however has a high level of sequence identity with a sequence neighbour of known three-dimensional structure (as identified by BLAST analysis of the E. coli genome). The insert of 3A12 spans residues 52 to 80 in the putative transport periplasmic protein (gene identifier 1787590) sharing a 48% sequence identity with residues 30 to 58 of the Salmonella oligopeptide-binding protein. In its 3D structure (Tame et al. 1994) these residues form a helix and two short antiparallel β-strands. The oligopeptide-binding protein as a mixed α/β protein has no structural homology with CspA and its residues 52 to 80 do not form part of a β-barrel. Thus sequences from different folds are juxtaposed in the chimeric protein 3A12. Thus while gene fragments selected from both libraries appear to be enriched for sequences forming primarily β-structure in their parent protein, polypeptide sequences originating from different folds are also represented.

### Example 13

Effects of Modified Selection Conditions

Proteolytic selection seemingly favoured phages displaying chimeric proteins with higher folding stabilities than those displaying chimeras with high melting points. From the plasmid-derived library the phage clone displaying the more stable protein A1 was selected twice as frequently as the less stable D6, which however has the higher melting point (Table I). In case of the genomic library the phages displaying the two most stable proteins (1C2, 1G6) were found four and five times, while the phages of the two less stable proteins (1B11, 2F3) were only found twice each after selection. Again the His-1b11 and His-2f3 proteins have the higher melting points (Table I). This suggests that escape from proteolysis depends more on stability than on the melting point as long as proteolysis is performed at temperatures well below melting points. Higher proteolysis temperatures than used here can therefore allow more frequent selection for proteins with higher melting points, while energetically more stable proteins would probably be enriched if phages are proteolysed for longer.

Such modified conditions can increase the frequency with which polypeptides exhibiting stabilities of natural proteins are selected from random combinatorial libraries. Further improvements can be expected by use of much larger repertoires, for example created by scale up, by improvements in the transfection efficiency of plasmid, phagemid or phage replicons into cells, or by other techniques such as in vivo recombination using the cre-lox system (Sternberg & Hamilton 1981). Alternatively or in addition repertoires could be further diversified by mutagenesis before or after selection. Effective repertoire sizes can further be increased, when recombination partners are enriched prior to recombination for in frame, no stop codon containing DNA fragments.

The presented methodology allows the selection of new chimeric proteins, which have been created through recombination of natural genes and which can combine properties from different molecules. Using suitable combinatorial partners, polypeptides can be created which inherit desirable functions (such as a target binding sites or an antigenic epitope) from parent proteins, while removing undesirable properties (such as unwanted receptor binding sites or unwanted epitopes). For this purpose, proteolytic treatment can be combined with selection for binding.

In the case of selection for binding of chimeric proteins to a ligand, it can be advantageous to increase the copy number of phage displayed fusion proteins. An increased copy number of displayed p3-fusion proteins, of which there can be up to five on each phage particle, would result in multiple binding events for a single clone, which can allow selection even in the case of chimeric proteins with a low affinity to the ligand. Copy number of fusion proteins in phage display can for example be increased, when phagemid-encoded fusion p3-fusion protein are rescued for phage preparation with a helper phage lacking the gene for p3 (Rakonjac et al. 1997)

### Example 14

Secondary Modifications of Selected Chimaeras

The binding activity of chimeric proteins created through the random recombination of polypeptide segments for a given ligand can be low, even if the parent proteins of these segments have a high affinity for such a ligand. Thus any newly juxtaposed polypeptide segment is expected to have some effect on the structure of the other when compared with its structure in the parent protein. As a consequence most binding sites will no longer fit a ligand with the same precision and result in a reduced affinity. It is therefore envisaged that it can be necessary to improve such binding sites, once a new chimeric protein has been created as part of a combinatorial library.

Improvements of selected chimeric proteins can be achieved by secondary modification or mutation. Such modifications can be made to improve binding, they can also be made to increase stability and/or to introduce new binding or enzymatic functions. The type of modification and its location in the chimeric protein (i.e., which old amino acid is replaced with which new one) can be based on rational design principles or partially or entirely random. Modifications can be introduced by a site-directed mutagenesis (Hutchison III et al. 1978) or by a site-directed random mutagenesis (Riechmann & Weill 1993) followed by selection or screening for activity or stability in the resulting mutant chimaeras. Alternatively an entirely random mutagenesis (through for example error-prone PCR amplification, Hawkins et al. 1992) of either one or both segments (or indeed their linking sequence) of the chimeric protein or through passage of the phagemid through an E. coli mutator strain (Low et al. 1996) followed by selection and/or screening for binding, enzymatic activity or stability.

Modifications can further comprise the deletion of residues or introduction of additional residues. In particular the joining and end regions of the recombined polypeptide segments can be expected to be not optimised. The joining regions can strain interactions between the juxtaposed segments, which can be relieved by introducing additional residues within the joining region. Regions close to the end of the chimeric protein can comprise terminal residues not participating in the fold of the domain, and their deletion can improve the overall integrity of the protein.

We demonstrate for one of the chimeric proteins how its stability was improved based on rational design. His-2f3 was created through the combinatorial shuffling of the N-terminal half of the E. coli protein CspA with random amino acid segments encoded by fragments of the E. coli genome (Example 1). The sequence and genetic origin of the random fragment are given in Table II. The spectroscopic analysis of His-2f3 (Example 7) indicates a fold rich in β-structure. If His-2f3 folds (like CspA) into a β-barrel certain sequence requirements may have to be met to improve the stability of the barrel.

In CspA the hydrophobic side chain of residue Leu45 closes one end of its β-barrel and Gly48 and Gln49 form a turn between two β-strands in the polypeptide fold to allow the formation of backbone hydrogen bonds of the following P-strand with the N-terminal β-strand of CspA. Within this strand the side chains of three hydrophobic residues (Val51, Phe53 and Ile55) point to the inside of the barrel. His-2f3 does not meet those requirements exactly but has a similar motifs within its genomic segments, as the residues Pro58, Gly61, Ala62, Met64, Phe66 and Ala68 (in its genomic segment) exhibit the same spacing as the motif described for CspA (compare Table III).

We therefore mutated the genomic segment in 2f3 at positions 58 (P to L), 62 (A to Q) and 68 (A to L) to match the amino acid types described for the motif in CspA, while the residues at position 61, 64 and 66 in 2f3 were already judged to be identical or similar enough. As summarised in Table III, the combined P58L and the A62Q mutations increased the stability of 2f3 to 6 kcal/mol, which lies within the range of typical natural protein domains and is 1.6 , kcal/mol higher than that of CspA itself. The A68L had no positive effect in 2f3.

In addition, the two C-terminal residues (PW) in 2f3 (compare Tables II and III) were removed, which were partially degraded in the originally expressed, soluble 2f3 protein. The removal of these residues had no significant effect on the overall stability of 2f3, but resulted in a more homogeneous protein preparation after expression, which for example is advantageous for structural studies like NMR.

This result shows that new chimeric proteins can be improved upon after selection through further modifications, in this case based on rational design.

### Example 15

Crossreactivity of Anti-CspA Antisera with Chimeric Proteins

A possible application of chimeric proteins is their use as vaccines against the parent polypeptide of one or more of the recombined amino acid sequences. For this purpose antisera against the chimeric protein will be cross-reactive with the parent polypeptide (and indeed vice versa).

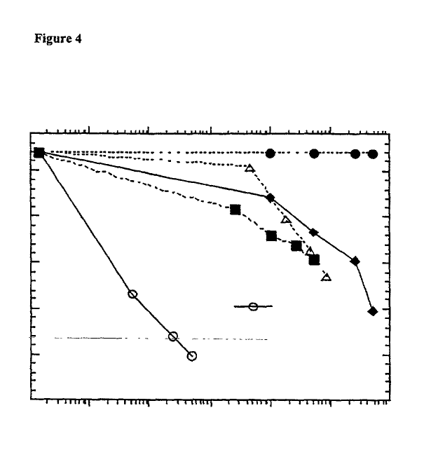

A rabbit was immunised with CspA using Freunds adjuvant (see Methods). The resulting antiserum recognised immobilised, biotinylated CspA. Binding of the rabbit antiserum to the immobilised Biotin-CspA could be competed with soluble CspA and to varying degrees with the chimeric protein His-1c2, His-2f3 and His-1b11 (FIG. 4).

To determine the crossreactivity of the final antiserum against CspA (serum 4156-4) with chimaeric polypeptides from the combinatorial library (Examples 1 and 3), antiserum 4156-4 was tested for binding to phages displaying various selected and unselected fusion proteins. Phages displaying CspA, the N-terminal half of CspA (CspA/2), selected chimaeric proteins 2f3, 1c2, 1b11, D6 or nothing (VCS-M13) were bound to the immobilised antiserum from a rabbit (taken after the third CspA immunisation boost) and detected with anti-M13 antibody conjugated HRP. The antiserum was immobilised with a biotinylated goat anti-rabbit antiserum on a Streptavidin coated ELISA plate. As expected, phage displaying intact CspA bound most strongly to the immobilised antiserum. Weaker but significant binding was also observed for phage displaying the N-terminal half of CspA only (CspA/2), which was used as a constitutive partner in the combinatorial library, or to varied degrees for the previously selected chimaeric proteins. Among these most strongly bound were the chimaeras D6, which comprises duplicated CspA sequences (Example 9), and 2f3, which has presumably a structural fold similar to that of CspA (see Example 14).

The polypeptide comprising CspA/2 does not form stably folded domains, as it is not proteolytically resistant (FIG. 1). Binding to the anti-CspA antiserum is therefore most likely due to binding to antibody combining sites specific for linear epitopes within the N-terminal half of CspA or to the ability of the flexible polypeptide to adopt the conformational CspA epitopes when bound to antibody.

It cannot be excluded that the binding of chimaera-phages to the antiserum is also partially due to the representation of linear CspA epitopes. However, as discussed in Example 16, antiserum binding of chimaera 2f3 appears to involve the recognition of conformational epitopes. The folded nature of 2f3 may indeed be responsible for a weakened binding to antibodies specific for linear CspA epitopes when compared to CspA/2, as it may be less flexible and therefore less able to adapt to those antibody combining sites. The very low binding of the folded chimaera 1c2 is probably also due to its inability to bind to antibodies specific for linear CspA epitopes, as it forms the most stable fold amongst the chimaeras.

This result shows that an immunisation with CspA results in an immune response which contains antibodies that crossreact with all three of the analysed chimaeras. Conversely, it should therefore also be possible to achieve an immune response against CspA when any one of the chimaeras is used for vaccination. The immune response can be expected to be directed against both linear and against conformational determinants of CspA.

### Example 16

Crossreactivity of Affinity Purified Anti-CspA Antibodies with Phages Displaying Chimaeric Proteins

The results from Example 15 suggest that the antiserum against CspA comprises a significant proportion of antibodies specific for linear CspA epitopes. To analyse this further, the antiserum 4156-4 was fractionated through binding to folded Biotin-CspA immobilised to Streptavidin-agarose to enrich for those antibodies specific for conformational determinants of CspA. This yielded the purified CspA-specific fraction E2 of rabbit antibodies (IgG).

Binding of phages displaying chimaeric protein domains or control polypeptides to the anti-CspA fraction E2 was compared with binding to the unfractionated antiserum 4156-4. Phages displaying CspA, CspA/2 or selected chimaeric proteins 2f3, 1b11 and 1c2 were bound to the immobilised rabbit antiserum 4156-4 or its affinity-purified fraction E2 and detected with anti-M13 antibody conjugated HRP. The antiserum or purified antibody fraction was immobilised with a biotinylated goat anti-rabbit antiserum on a Streptavidin coated ELISA plate. While under the conditions of the ELISA all phages were bound better by the E2 fraction than by the unpurified antiserum, binding of phages displaying CspA and 2f3 was significantly more increased than that of phages displaying CspA/2 or the chimaeras 1b11 and 1c2. Relatively (to CspA/2) improved binding of intact CspA confirms that the affinity purification lead to the enrichment of antibodies recognising folded CspA rather than linear epitopes therefore.

Among the chimaeric proteins, only the binding of 2f3 to fraction E2 was improved in a way similar to that of CspA. This suggests that 2f3 is able to bind to more conformational CspA epitopes than the two other chimaeras. Chimaeric protein 2f3 may therefore serve as a good ‘model’ vaccine able to raise antibodies specific for conformational epitopes of the parent protein CspA.

### Example 17

Crossreactivity of Anti-CspA Antisera with Chimaeric Proteins Comprising Duplicated Sequence Segments

As described in Example 9, the chimaeric protein D6 selected by proteolysis from the plasmid-derived library (Example 4) comprises a partial duplication of the N-terminal half of CspA. The reactivity with the purified anti-CspA antibody fraction E2 with phage displaying the chimaera D6 was compared with that of phages displaying other proteins.

D6 phage is highly reactive with the antiserum 4156-4, indeed more than all chimaeras selected from the genomic library including 2f3. D6 is roughly as stable as 2f3 (Table I) and comprises in its C-terminal part residues 17 to 53 from CspA. The presence of additional CspA amino acid sequences alone may explain why it reacts so strongly with the anti-CspA antibodies. Duplicated sequences may in addition allow a single antibody to bind with both of its binding site to the same D6 molecule creating an avidity effect for phage binding in the ELISA. These results suggest that chimaeric proteins comprising duplicated amino acid sequence segments may be particularly useful for the creation of vaccines to direct the immune response to specific and preferably conformational epitopes.

### Example 18

Crossreactivity of Anti-CspA Antisera with Soluble Chimaeric Proteins

In examples 15 to 17 the crossreactivity of various phage-displayed chimaeric proteins with the anti-CspA antiserum and affinity purified anti-CspA antibody fraction is described. However, to be used as a therapeutic reagent it may be advantageous and more effective to use the isolated chimaeric protein instead of a phage particle displaying the chimaeric protein. Therefore, the binding of soluble, purified chimaeric proteins to the anti-CspA antiserum was analysed.

A rabbit anti-CspA antiserum was incubated with varied amounts of soluble His-CspA, His-1c2, His-2f3, His-1b11 or lysozyme (as a negative control) before binding to biotinylated CspA immobilised on Streptavidin coated ELISA well. Bound rabbit antisera were detected with a 10 HRP-conjugated goat anti-rabbit IgG antiserum. Binding of the anti-CspA antiserum to immobilised Biotin-CspA could be competed with soluble CspA and to varying degrees with the chimaeric protein His-1c2, His-2f3 and His-1b11. This result shows that an immunisation with CspA results in an immune response which contains antibodies that crossreact with all three of the analysed chimaeras. Conversely, it should therefore also be possible to achieve an immune response against CspA when these chimaeras are used for vaccination. In this respect, the chimaeric protein domain 2f3 is particularly promising, as it is clearly the chimaeric domain most reactive with especially the purified antiserum fraction E2 (see above). An immune response against 2f3 can be expected to be directed against both linear and against conformational determinants of CspA.

### Example 19

Selection of Chimeric Proteins for Binding

In the earlier examples, stably folded chimeric domains were selected by proteolysis through the combinatorial juxtaposition of the N-terminal half of the E. coli protein CspA with amino acid segments encoded by fragments of the E. coli genome (Examples 1 and 3). A number of these chimeric proteins are expected to form a polypeptide fold resembling that of CspA as the secondary structure prediction and spectroscopic analyses of the four chimaeras described (Example 7) indicates a fold rich in β-structure.

It is possible that the RNA binding function (Jiang et al. 1997) of CspA is retained in some of the selected chimaeras. The nucleic acid binding site in CspA has been proposed to be located on a surface formed around Trp11, Phe18, Phe20, Phe31 and Lys60 (Newkirk et al. 1994; Schroder et al. 1995). While the four aromatic residues are part of the N-terminal half of CspA and are therefore present in all members of the genomic repertoire (Example 1), residue Lys60 is not. It seems likely that in some of the chimeric proteins the nucleic acid binding activity will be retained; such proteins could be selected for example by binding of phage displaying the protein to nucleic acid immobilised on solid phase. (However, the phage display system used in the experiments above would be unsuitable as the barnase tag retains nucleic acid binding activity).

Furthermore, for functional selection it can be useful to use a phage-display system which allows the multiple display of the fusion protein thereby facilitating selection of chimeric proteins with low affinities for the ligand (in this case nucleic acid) through the resulting avidity effect. This can be achieved in the case of chimaeras fused to the phage coat protein p3 for example through the use of a phage vector like phage fd (Zacher et al. 1980), through the use of a phagemid in 1 combination with a helper phage devoid of the phage p3 gene (Rakonjac et al. 1997) or through an increased expression of functional chimaera-p3-fusion protein. Alternatively, multiple display can be achieved through fusion to a different phage coat protein, like p8.

### Example 20

Selection of Chimaeric Proteins for Folding and Binding to Antibodies

Of particular importance is the binding of the chimeric domains to antibodies. If antiserum against the parent protein were used for selections, this would be expected to direct the selection to any of the epitopes of the chimeric protein that are similar to those in the parent protein and are represented in the anti-serum. Alternatively monoclonal antibodies could be used which would select for those clones binding a single epitope that is similar to that of the parent protein. A number of these chimeric proteins are expected to form a polypeptide fold resembling that of CspA, as the secondary structure prediction and spectroscopic analyses of the four chimaeras described in Example 7 indicates a fold rich in β-structure. If any of the recombined chimeric proteins within the repertoire resemble in fold that of CspA, it should therefore be possible to enrich for such proteins through binding to antibodies which specifically recognise CspA.

Example 15 already describes that an anti-CspA antiserum crossreacts with three of the chimeric proteins selected through proteolysis (and barstar binding) alone. The anti-CspA antiserum can therefore serve as a reagent to enrich the combinatorial library from Example 1 specifically for phages displaying chimeric proteins which resemble CspA most closely.

A rabbit anti-CspA serum was fractionated through binding to Biotin-CspA immobilised to Streptavidin-agarose to enrich for that against conformational determinants of CspA. Purified CspA-specific (anti-CspA) rabbit antibodies (IgG) were tested for anti-CspA binding activity as described in Example 15. For use in phage selection the anti-CspA rabbit antibodies were immobilised on a Streptavidin-coated ELISA-well plate through a commercial biotinylated goat anti-rabbit IgG antiserum. Phages (7×10⁹ cfu) from the genomic library of chimeric proteins (Example 1), which had undergone one round of proteolytic selection (followed by barstar binding, see Example 3), were treated with trypsin and thermolysin (see Example 3) followed by binding to the CspA-specific rabbit antibodies in 2% BSA in PBS. After washing with PBS and 40 mM DTT 4.3×10³ bound phage were eluted at pH 2, neutralised and used for infection of bacterial cells.

Ninety-six (96) of the resulting clones were grown up in a multiwell plate and infected with helper phage KM13 for phage production. Phage from the culture supernatants from the infected bacterial clones were bound to the anti-CspA antibodies, which again had been immobilised to a Streptavidin-coated plate via a biotinylated goat anti-rabbit IgG antiserum. Bound phage was washed with PBS, exposed to trypsin and thernolysin after immobilisation as before, washed with PBS, and remaining phage was detected with an anti-M13-HRP conjugate. Sequences of the nine clones with the strongest signal remaining after proteolysis were determined. Seven of these clones were identical (two) or almost identical (five clones had one residue less at the N-terminal end of the genomic insert and two different residues at the C-terminal end) to the clone 2f3, which had been previously selected-albeit not at the same high frequency-after proteolytic selection/barstar binding (Examples 3 and 4). The two remaining sequences had not been previously observed. Purified phage of the 2f3 and 2f3-like clones was confirmed to be strongly reactive with the purified rabbit anti-CspA antibodies, also after exposure to trypsin in solution, confirming that it is protease-resistant folded sequences that are binding to antibody. Together with the fact that the anti-serum had been fractionated for binding to the folded CspA, this indicates strongly that the selection has been towards a conformational determinant. The ELISA in FIG. 4 (see Example 15) proves that the corresponding chimeric protein also interacts in its soluble version with an anti-CspA antiserum.

This experiment shows that it is possible to identify “isosteric” peptides (same conformation in parent protein and chimeric domain). It also indicates that the method can be used for vaccination towards a conformational segment of the protein; thus it should equally be possible to use 2f3 for vaccination and to produce anti-serum that recognises the conformation of the N-terminal portion of CspA.

### Example 21

Immunisation and Vaccination with Selected Chimaeric Proteins

The purified chimaeric proteins His-2f3 and His-1c2 (see Examples 4 to 6) were used for immunisation (initial immunisation using the chimaeric protein mixed with Freund's adjuvant followed by three boost only with protein in PBS) of a rabbit to analyse if resulting antisera from the immunised animals are crossreactive with CspA. The animals were then challenged with an injection of folded CspA (in PBS) to see if a specific anti-CspA immune response involving T cell mediated help was established during immunisation.

Immunizations and challenges followed the following scheme. A rabbit, was immunised and boosted three times with His-2f3 (2^{nd}, 3^{rd}, 4^{th} vaccination) before being challenged with CspA (1^{st} CspA injection), and another rabbit was immunised and boosted three times with His-1c2 (2 d, 3d, 4 h vaccination) before being challenged with CspA (1^{st} CspA injection). Phages displaying CspA, CspA/2 (the N-terminal half of CspA only) or the chimaeric proteins 2f3 and 1c2 were bound to immobilised antisera from the rabbits. Bound phage was detected with an anti-M13 antibody-HRP conjugate. For comparison, the antisera of both rabbits before immunisation were also tested, as well as the antisera from a third rabbit (immunised with CspA) taken after the first injection and second injection with CspA (1^{st}, 2^{nd} CspA injection). The antisera were immobilised with biotinylated goat anti-rabbit antibodies on a Streptavidin-coated ELISA plate.

The analyses of the rabbit immune response show that immunisation with both 2f3 and 1c2 raised antisera highly reactive with their respective antigen as they bound phage displaying these chimaeric proteins strongly after the second, third and fourth vaccination. Crossreactivity with CspA (when displayed on phage) was observed for both animals.

Crossreactivity is stronger with phage displaying the N-terminal half of CspA (CspA/2) only. As the CspA/2 on its own is largely unfolded (FIG. 1), the crossreactivity between CspA and CspA/2 is most likely due to shared linear epitopes. These will be less abundant (if at all present) in the chimaeric domains 2f3 and 1c2, which are stably folded (Example 7; FIG. 1). However, for vaccination it is most important how the immune system of the immunised organism reacts to challenge with the real pathogen. Thus if His-2and His-lc2 are ‘model’ vaccines, CspA would be the ‘model’ pathogen, and the reaction of both rabbits to a challenge with CspA would be the critical test for the vaccination experiment. While for the rabbit, which was immunised His-1c2, very little antibody response was observed after injection of purified intact CspA (in PBS), the rabbit, which was immunised with His-2f3, showed s strong antibody response to CspA. Thus the antiserum from the 2f3-rabbit taken after a single injection of CspA was now strongly reactive with CspA-displaying phage, while the that taken from the 1c2-rabbit after CspA challenge was not. The anti-CspA immune response of the challenged 2f3-rabbit was indeed comparable with that of a rabbit, which had been immunised and then boosted once with CspA itself.

The increased reactivity to CspA observed for the 2f3-rabbit after CspA challenge indicates that a significant number of its B memory cells, resulting from the immunisation with His-2f3, must express anti-2f3 antibodies, which recognise CspA and which are specifically activated after the CspA challenge. CspA and 2f3 must therefore share identical B and T cell epitopes, leading to specific T cell helper activation of the same memory B cells by both CspA and 2f3. Further, as a significantly smaller increase was observed for the reactivity of the same antiserum with phage displaying only the N-terminal (and presumably largely unfolded) half of CspA, much of the anti-CspA response must be due to the recognition of conformational epitopes.

His-2f3 must therefore be judged to be a successful ‘model’ vaccine for the ‘model’ pathogen CspA, as it was able to induce a cell-mediated, specific immune response, which furthermore seems to involve the recognition of conformational B cell epitopes.

### Example 22

Selection of Chimaeric Proteins Through Binding to Antibodies Alone

Example 20 demonstrates the proteolytic selection of stably folded proteins from the genomic combinatorial library (Example 1) followed by selection for binding to affinity-purified antibodies (fraction E2) from an animal immunised with one of the parent proteins donating an amino acid segment to the chimaeric protein has lead to the isolation of a stably folded chimaeric protein, which shares epitopes, of which at least some are conformational, with this parent protein.

The same combinatorial library (Example 1) was also selected simply for binding to the antiserum fraction E2 (as used in Example 20) without selection for proteolytic stability. Initially the library was enriched for phages displaying fusion proteins by capture to biotinylated barstar alone (see Example 3). The resulting pool of phage displayed library was bound to biotinylated E2-antibodies immobilised on a Streptavidin coated plate and eluted with DTT, which leads to elution of antibody bound phage from the well through cleavage of the disulphide linked biotin label of the antibody. This selection was repeated four times. In the first two rounds phage was treated after elution with trypsin to remove protease-sensitive helper-phage derived infection-proteins leading to a background infectivity of phage not-displaying a fusion protein. In the last two rounds eluted phage was rebound to a second Streptavidin-well coated with biotinylated barstar, washed and eluted with 20 mM glycine, pH 2. This step was, like the trypsination of the DTT-eluted phage in the first two rounds, designed to remove any phage without bamase-g3p fusion protein non-specifically carried along during selection. Due to the lower number of rescued phage using the barstar binding in the latter rounds, this method is probably more effective in reducing background. Otherwise selection was performed as in Example 3.

After the third and fourth round of selection 96 isolated phage clones were tested for binding to immobilised E2 antibodies. All phages showed specific binding, and the 14 strongest binder from both rounds were sequenced (not shown). 28 different sequences were found and still bound E2 antibody strongly after purification of phage by PEG precipitation. However, all tested phages lost more than 90% of barstar binding activity after exposure to trypsin and thermolysin. This suggests that the chimaeric proteins may be largely flexible (and therefore highly susceptible to proteolytic attack). Based on this assumption their reactivity with the E2-antibodies may in many cases be due to binding of linear epitopes presented by the chimaeras and/or the ability of flexible chimaeras to adapt to antibody combining sites specific for conformational epitopes (i.e. the chimaeras are able to adopt a CspA-like conformation on the antibody, but are not stably folded in the absence of antibody).

To study the behaviour of the soluble chimaeric proteins selected through binding to E2antibodies alone, the pool of their genes was amplified by PCR and subcloned for expression into a cytoplasmic expression vector pLR97. In this vector the chimaeras are fused with an N-terminal 6× His tag and a C-terminal peptide tag recognised by the antibody M2. The pool of chimaeras in this expression vector was transfected into E. coli and 96 clones were tested for expression of soluble protein. This was achieved through capture of the cell lysate on M2 (anti-peptide tag) antibody bound to a Streptavidin-coated ELISA well, washing and detection of bound chimaeric protein with E2-antibodies, which themselves were detected with HRP-conjugated anti-rabbit antibodies.

Six out of sixteen clones, which gave the strongest signal, were found to express soluble chimaeric proteins, which remained intact after purification on NTA-agarose via the His-tag. The other clones were proteolysed before or during purification. The six clones, which were able to evade proteolysis in the expressing E. coli cells, must present some structural features and cannot be of an entirely random coil nature. All six purified proteins still reacted strongly with the E2-antibodies and should therefore, when used in vaccination, be able to raise an immune response, which is able to react to the CspA protein.

However, it may be advantageous to screen the selected chimaeric proteins, which are expressed in a soluble and intact form in E. coli, for those most stable and best folded (for example using assays for in vitro proteolytic resistance, co-operative folding or reduced binding to 1-anilinonaphthalene-8-sulfonate) or for those that have specific binding features (for example better binding to antisera fraction E2 than to the unpurified antiserum; no reaction with a monoclonal antibody for a linear CspA epitope; binding to a monoclonal antibody specific for conformational CspA epitope).

Methods (for Examples 1 to 22)

Vector Constructions

The gene for the H102A mutant of barnase (Meiering et al. 1992) was fused to the N-terminus of the gene 3 protein (p3) of phage fd (Zacher et al. 1980) in a modified phagemid pHEN1 (Hoogenboom et al. 1991) between the DNA encoding the pelb leader peptide and the mature p3 after PCR amplification with suitable oligonucleotides using NcoI and PstI restriction sites to create the vector p22-12. Into p22-12 suitably amplified parts of the E. coli gene CspA (Goldstein et al. 1990) were cloned between the barnase and the p3 genes using PstI and NotI restriction sites. In the resulting phagemid vector pC5-7 the barnase gene is followed by the N-terminal 36 residues of CspA (the N-terminal Met being mutated to Leu to accommodate the PstI site) and the DNA sequence GGG AGC TCA GGC GGC CGC AGA A (SEQ ID NO: 1; SacI and NotI restriction sites in italics) before the GAA codon for the first residue (Glu) of p3. In pC5-7, the bamase-Csp cassette is out of frame with the p3 gene. In the control vector pCsp/2 the barnase-Csp cassette is in frame with the p3 gene, but the first codon of the linking DNA constitutes an opal stop codon.

Vectors for the cytoplasmic expression of soluble proteins were constructed by subcloning of genes from the phagemids into the BamHI and HindIII sites of a modified QE30 vector (Qiagen). This vector is identical to QE30 except for a tetra-His tag. During PCR-aided subcloning using the primers CYTOFOR (5′-CAA CAG TTT AAG CTT CCG CCT GAG CCC AGG-3′; SEQ ID NO: 2) and CYTOBAK (5′-CCT TTA CAG GAT CCA GAC TGC AG-3′; SEQ ID NO: 3) opal stop-codons were converted into the Trp-encoding TGG triplet.

Library Construction

As templates for random amplifications 100 ng of a pBCSK (Stratagene) based plasmid containing the entire CspA coding region or genomic DNA (2 μg digested with SacI) from the E. coli strain TG1 (Gibson 1984) prepared as described (Ausubel et al. 1995) was used in 25 PCR cycles with an annealing temperature of 38° C. using the oligonucleotide SN6NEW (5′-GAG CCT GCA GAG CTC AGG NNN NNN-3′; SEQ ID NO: 4) at 40 pmole/ml for the plasmid or in 30 PCR cycles with an annealing temperature of 38° C. using the oligonucleotide SN6MIX (5′-GAG CCT GCA GAG CTC CGG NNN NNN-3′; SEQ ID NO: 5) at 40 pmole/ml for the genomic DNA. PCR products were extended in a further 30 cycles with an annealing temperature of 52° C. using the oligonucleotide NOARG (5′-CGT GCG AGC CTG CAG AGC TCA GG-3′; SEQ ID NO: 6) at 4,000 pmole/ml for the plasmid and the oligonucleotide XTND (5′-CGT GCG AGC CTG CAG AGC TCC GG-3′; SEQ ID NO: 7) at 4,000 pmole/ml for the genomic DNA. PCR products of around 140 bp were purified from an agarose gel and reamplified in 30 PCR cycles using the oligonucleotide NOARG at an annealing temperature of 50° C.

Resulting fragments were digested with SacI, purified and ligated into the phosphatased and SacI-digested vector pC5-7. Ligated DNA was electroporated into TG1 creating a plasmid-derived repertoire of 1.7×10⁸ clones and a genomic repertoire of 1.0×10⁸ clones. In both libraries about 60% of the recombinants contained monomeric inserts, while the remainder contained oligomeric inserts. Ligation background was less than 1% for both ligations. Due to differences in the 3′ end of the PCR primers XTND and NOARG 40% of clones with in-frame inserts in the genomic library contained a GGA-encoded Gly residue as part of the 3′-SacI site, while the remaining clones contained the TGA-encoded opal stop-codon at the same position. All members of the plasmid-derived library with in-frame inserts contained the TGA-encoded opal stop-codon at this position.

Selections

For selections about 10¹⁰ colony forming units (cfu) of phage were treated with 200 nM trypsin (Sigma T8802) and 384 nM thermolysin (Sigma P1512) in TBS-Ca buffer (25 mM Tris, 137 mM NaCl, 1 mM CaCl₂, pH 7.4) for 10 minutes at 10° C. Proteolysed phage was captured for 1 hour with biotinylated C40A,C82A double mutant bamase inhibitor barstar (Hartley 1993, Lubienski et al. 1993) immobilised on a streptavidin coated microtitre plate (Boehringer) wells in 3% Marvel in PBS. Wells were washed twenty times with PBS and once with 50 mM dithiothreitol (DTT) in PBS for 5 minutes to elute phage containing proteolysed p3-fusions held together solely by disulphide bridges. Bound phage was eluted at pH 2, neutralised to pH 7 and propagated after reinfection.

For selection through antibody binding, initially about 10¹¹ colony forming units (cfu) of phage were bound to an immunotube (Nunc) coated with biotinylated barstar in 3% Marvel in PBS. The tube was washed twenty times with PBS and bound phage was eluted at pH 2, neutralised to pH and propagated after reinfection. The resulting phages comprised to 90% chimaeric fusion genes, which were in frame with barstar and g3p and contained no stop-codons (in addition to the opal-stopcodon at the C-terminal end of the fusion gene). About 10¹⁰ colony forming units (cfu) of such phage was then captured for 1 hour with the biotinylated antiserum fraction E2 (see below) immobilised on a streptavidin coated microtitre plate (Roche) washed as above and eluted with 200 μl of 50 mM DTT. The E2 fraction had previously been biotinylated with the DTT-sensitive reagent Biotin-disulphide-N-Hydroxysuccinimide (Sigma B-4531). The eluted phage was then incubated for 10 minutes at room temperature in 800 ml TBS-Ca containing 0.3 ng/ml trypsin and 5 ng/ml thermolysin. The mixture was then combined with 13 ml of exponentially growing E. coli cells TG1 for infection. This selection was repeated once more and the followed by two rounds of selection, in which antibody-captured and DTT-eluted phage was recaptured on a barstar-coated streptavidin ELISA well before elution at pH 2.

Phage ELISA

Proteolysis and binding of purified phage (about 10¹⁰ cfu per well) to immobilised barstar was performed as above. Phage remaining bound after washes with PBS and DTT was detected in ELISA with an anti-M13 phage antibody-horse radish peroxidase (HRP) conjugate (Pharmacia) in 3% Marvel in PBS. Non-purified phage from culture supernatants was bound to the biotinylated barstar and then proteolysed in situ. Purified phage was proteolysed in solution and proteases were inactivated with Pefabloc (Boehringer) and EDTA before capture.

Anti-CpsA Antisera

A first anti-CspA serum was obtained from an immunised rabbit. The rabbit was injected once with refolded (see above) His-CspA (0.5 ml at 1.75 mg/ml PBS) mixed with 1:1 with Freund's complete adjuvant, followed by two injections with refolded His-CspA (0.5 ml at 1.75 mg/ml PBS) mixed 1:1 with Freud's incomplete adjuvant in 4 week intervals to boost the immune response. The antisera used was obtained from blood taken ten days after the second boost.

A second anti-CspA serum (serum 4156 as used for Example 15 and for purification of anti-CspA specific antibodies in Example 16) was obtained from a different immunised rabbit. The rabbit was injected once with refolded (see above) His-CspA (0.5 ml at 1.75 mg/ml PBS) mixed with 1:1 with Freund's complete adjuvant, followed by three injections with refolded His-CspA (0.5 ml at 1.75 mg/ml PBS) alone in 4 week intervals to boost the immune response. The antisera used were obtained from blood taken before immunisation or ten days after the third boost. One ml of this antiserum was purified on 0.2 ml of Streptavidin-agarose (Pierce No. 53117), to which about 0.1 mg Biotin-CspA (see below) was bound, after washing with PBS, elution at pH 2 followed by neutralisation and buffer exchange into 3.5 ml PBS (i.e., at 3.5 fold dilution compared to the original antiserum). When used for phage selection, the purified anti-CspA antibodies were 500-fold diluted in PBS for binding to a biotinylated goat anti-rabbit antiserum (Sigma B-7389) immobilised in Streptavidin-coated ELISA wells.

Further, one rabbit each was injected with His-2f3 or His-1c2 (0.5 ml at 1.5 mg/ml PBS) mixed 1:1 with Freund's complete adjuvant, followed by three injections with His-2f3 or His-1c2 (0.5 ml at 1.5 mg/ml PBS) alone in 4 week intervals to boost the immune response. Antisera samples from the immunised animals were taken before immunisation and 14 days after each of the boost injections. Four weeks after the third boost both animals were injected with refolded CspA (0.5 ml at 1.5 mg/ml in PBS) alone. A final antiserum was taken 14 days after the CspA injection.

Refolding and Biotinylation of CspA

His-CspA, as used for immunisation and data in Table III and FIG. 4, was purified from the unfractionated E. coli cell pellet using NTA agarose after solubilisation with 8M urea in TBS. Before elution with 200 mM imidazole in PBS, agarose bound His-CspA was renatured with an 8M to 0M urea gradient TBS. Eluted protein was dialysed against PBS.

For biotinylation, CspA was modified through addition of cysteine-glutamine-alanine residues as a C-terminal tag, introduced on the gene level using suitable PCR primers. The corresponding His-CspA-Cys protein was expressed, purified and refolded as His-CspA except for the addition of 0.5 mM DTT to all solutions. The NTA agarose with the bound His-CspA-Cys was washed with 5 volumes of PBS (all solutions without DTT from this step onwards) and mixed with the biotinylation reagent EZ-Link” Biotin-HPDP (Pierce) for biotinylation according to the manufacturer's instructions. After 1 hour the agarose with the bound and biotinylated protein was washed with 10 volumes of PBS, eluted with 200 mM imidazole in PBS and buffer-exchanged into PBS. Biotinylation of the now His-Biotin-CspA was verified by MALDI mass spectrometry using a SELDI (Ciphergen systems).

Binding ofHis-Biotin-CspA to the second rabbit anti-CspA serum (see above) during the immunisation protocol (FIG. 4) was analysed after immobilisation of the antisera on a Protein A (at 1 mg/ml) coated ELISA-plate (Nunc Maxisorb Immunoplate). His-Biotin-CspA bound in 1% BSA was detected with a streptavidin-HRP conjugate(Sigma).

Competitive His-CspA binding of the first rabbit anti-CspA antisera to CspA was analysed after immobilisation of biotinylated His-Csp-Cys (at 0.25 μg/ml in PBS) onto streptavidin-conjugated ELISA plates (Boehringer Mannheim). The rabbit anti-CspA serum(taken after the second boost) was diluted 1/30,000 in 2% bovine serum albumin in PBS and preincubated with varied amounts of purified competitors (see FIG. 4) before binding to the ELISA well. Bound rabbit antibodies from the serum were detected with a HRP-conjugated goat anti-rabbit IgG antiserum (Sigma).

Binding of phage displaying g3p-fusion proteins to the antisera was analysed after capture of the anti-CspA serum or its fraction E2 on a streptavidin-conjugated ELISA plate (Roche) via a biotin conjugate of a goat anti-rabbit IgG antiserum (Sigma B-7389). Phage bound in the presence of 1% BSA in PBS was washed with PBS and detected with an HRP-conjugated anti-M13 monoclonal antibody (Pharmacia). All fusion-protein phages, except phage displaying CspA, displayed the chimaera (or CspA/2) between bamase and g3p as described above. Thus amount of displayed fusion protein was adjusted using the ELISA signal for barstar binding. Phage comprising CspA as a g3p-fusion protein had CspA displayed between D2 and D3 of g3p (as in Kristensen & Winter 1997) and contained no bamase fusion protein. Its concentration in ELISA could therefore not be normalised based on barstar binding, and it was used at half the average concentration of the other phages (based on original bacterial culture).

2f3 Mutants

The gene for the 6H-2B protein (compare Table III) was prepared by PCR with the primers QEBACK (5′-CGG ATA ACA ATT TCA CAC AG-3′; SEQ ID NO: 8) and 2F3FOR (5′-GGC CGC CTG AAG CTTTTA AGG CGG ATG GTT GAA-3′; SEQ ID NO: 9) using the 2f3 gene in QE30 (compare Table II) as a template. Mutant genes for the 6H-2f3 protein were prepared through PCR amplification of the partial 2f3 gene using accordingly designed primers and the same template. For each mutant two PCR products (covering the N and C-terminal portion of the 2f3 gene respectively) were purified, denatured, annealed and extended. Full-length mutant genes were specifically reamplified using the two outside primers BACKTWO (5′-CCT TTA CAG GAT CC-3′; SEQ ID NO: 10) and 2F3FOR. Complete genes were digested with HindIII and BaniHI and cloned into the unmodified QE30 vector (Qiagen; encoding a 6 histidine containing N-terminal tag).

For the mutant 6H-2f3-P58L the primers 2F3F2 (5′-GGT AAA AAG CAT GAT TGC GCC AAT TTC TAG CTC GCC TGC-3′; SEQ ID NO: 11), CYTOBAK (for the N-terminal half), 2F3B0 (5′-GGT AAA AAG CAT GAT TGC G-3′; SEQ ID NO: 12) and QEFOR (5′-GTT CTG AGG TCA TTA CTG G-3′; SEQ ID NO: 13) (for the C-terminal half were used). For the mutant 6H-2f3-P58L,A62Q the primers 2F3F1 (5′-GGT AAA AAG CAT GAT TTG GCC AAT TTC TAG CTC GCC TGC-3′; SEQ ID NO: 14), CYTOBAK (for the N-terminal half), 2F3B0 and QEFOR (for the C-terminal half were used). For the mutant 6H-2f3-P58L,A62Q,A68L the primers 2F3F1, CYTOBAK (for the N-terminal half), 2F3B1 (5′-AAT CAT GCT TTT TAC CCT AAT GGA TGG C-3′; SEQ ID NO: 15) and QEFOR (for the C-terminal half were used).

Protein Expression, Purification and Analysis

Proteins were expressed by induction of exponential bacterial cultures at 30° C. and purified from the soluble fraction of the cytoplasm using NTA agarose according to the Qiagen protocol. His-1 g6 was purified after solubilisation with 8 M urea in TBS and refolded by dialysis from 8 M, 4 M, 2 M, 1 M, 0.5 M to 0 M urea in TBS. Proteins were further purified by gel filtration on a Superdex-75 column (Pharmacia). The molecular weight of proteolytic fragments was determined using the surface enhanced laser desorption/ionisation (SELDI) technique (Hutchens & Yip 1993).

Proteolysis of soluble proteins (about 40 μM) was carried out using 40 nM of trypsin, thermolysin or α-chymotrypsin (Sigma C3142) in TBS-Ca at 20° C. for 10 minutes. Circular dichroism spectra and thermodenaturation were recorded as described (Davies & Riechmann 1995). Thermodenaturation of 10 μM protein (His-1c2 at 2 μM) in PBS was followed at a wavelength between 220 nm and 225 nm (His-1c2 in 2.5 mM phosphate buffer, pH 7, at 205 nm). Nuclear magnetic resonance experiments were performed on a Bruker DMX-600 spectrometer as described (Riechmann & Holliger 1997) using a watergate sequence (Piotto et al. 1992) for water suppression with protein at 1 mM in 20 mM phosphate buffer at pH 6.2 containing 100 mM NaCl in 93% H₂O/7% D₂O or 99.9% D₂O.

Expression and Detection of Chimaeric Protein with a C-Terminal Flag-Tag

DNA from phage expressing chimaeric proteins was amplified using the primers CYTOBAK and CYTOFLAG (5′-CAG TTT CTG CGG AAG CTT GAG CCC AGG-3′). CYTOFLAG converts the C-terminal opal-stopcodon into a tryptophan-codon and introduces a HindIII restriction site (italics). Amplified chimaeric genes were restricted with BamHI and HindE and subcloned for cytoplasmic E. coli expression into the vector pLR97. This vector is based on QE30 (Qiagen) but adds to the C-terminal WAQAQ residues in the chimaeric proteins the peptide sequence DYKDDDDK (so-called Flag-tag), which is recognised by the monoclonal antibody M2 (Sigma F-3165).

For detection of expressed, intact protein recombinant clones in E. coli were grown at 37° C. and induced (using 1 mM IPTG) for 4 hours at 30° C. in ELISA wells. Induced cells were spun down and resuspended in B-PER Reagent (Pierce CC46339) for cell lysis (30 minutes shaking at room temperature. Lysate supernatants were captured on biotinylated M2 antibody (Sigma F-9291) in Streptavidin coated ELISA wells and bound chimaeric proteins were detected with the antiserum fraction E2 (see above) and an HRP-conjugated anti-rabbit antiserum (Sigma A-6154). Purification of chimaeric protein domains was performed as above.

### Examle 23

Immunogenic Polypeptides.

The amino acid sequences of chimeric proteins according to the invention can contain sequences designed to display epitopes for vaccination against the parent protein from which the amino acid sequences are derived. As mentioned above, this can be useful for vaccination against infection, so-called prophylactic vaccination, or for directing an immune response against a human protein (e.g., a T-cell epitope, a tumor antigen, etc.) as part of a therapeutic regimen.

An immunogenic polypeptide comprising a folded chimeric polypeptide according to the invention can be made as follows. For a prophylactic vaccine, a sequence segment from a protein expressed by a pathogen, e.g., a surface protein of a virus, such as influenza virus hemagglutinin (HA; multiple sequences are available in GenBank, e.g., AF186269, AF186268, AF186267, and AF186266 are each influenza virus HA sequences), is used as a constitutive partner in a combinatorial library of polypeptides generated through shuffling of this sequence with sequences from another genetic source, for example, random sequences taken from the E. coli genome. A 50 amino acid sequence is chosen from any point in the HA sequence, for example: KELLHTEHNG MLCATDLGHP LILDTCTIEG LVYGNPSCDL LLGGKEWSYI, which corresponds to amino acids 45 to 95 (nucleotides 135 to 285) of the influenza HA protein sequence of GenBank Accession No. AF186266. This sequence is expressly not chosen to contain any complete or discrete structural element or complete domain. The chosen HA coding sequence is fused to random sequences of the E. coli genome about 140 base pairs in size as used in Example 1 herein. The resulting chimeric sequences are fused to the coding sequence for the p3 bacteriophage coat protein in a vector for protein display on filamentous phage. An example of the resulting chimeric polypeptide sequence could be KELLHTEHNG MLCATDLGHP LILDTCTIEG LVYGNPSCDL LLGGKEWSYI GCVPYTNFSL IYEGKCGMSGGRVE GKVIYE TQSTHKHSW, which is the chosen HA fragment fused to the E. coli genomic fragment 12035 to 11927 in ECAE485 of the E. coli genome.

The resulting phage are then screened for those with stable folded structures by exposure to a protease in vitro as described herein. Phage expressing folded chimeric HA/E. coli domains are not cleaved by the protease. These phage are then screened for binding to an anti-HA antibody. A number of anti-HA antibodies, both polyclonal and monoclonal, are well known in the art (see for example, Daniels et al., 1987, EMBO J. 6: 1459-1465) and several are commercially available. If necessary, phage that are not proteolytically cleaved can be screened against a panel of antibodies, in order to identify those antibody preparations that bind a structure comprising the selected HA sequence. Phage expressing chimeric HA/E. coli polypeptide domains that are properly folded and bind the anti-HA antibody are propagated, and the chimeric polypeptide domain is identified. Because it binds the HA antibody, it will have structural similarity to an influenza virus epitope and is therefore useful as a vaccine component. The chimeric protein domain can be expressed on its own (or as a fusion with a carrier, such as glutathione-S-transferase) and used as an immunogen.

As an example of how one would make a chimeric folded polypeptide domain useful as a therapeutic vaccine, one can use nucleotide sequence encoding a sequence taken from a constant region of human IgE (e.g., WILFLVAAAT RVHSQTQLVQ SGAEVRKPGA SVRVS, corresponding to amino acids 5-40 of the human immunoglobulin E heavy chain sequence in GenBank Accession No. L00022, or, alternatively another random fragment of the polypeptide), linked to random genomic fragments from E. coli, as described above. A resulting fragment can comprise, for example, the sequence WILFLVAAAT RVHSQTQLVQ SGAEVRKPGA SVRVSLQSGK MTGIVKWFNA DKGFGFITPD DGSKDVFVHF SAGSS, which corresponds to the 35 amino acids derived from the human IgE with 40 amino acids derived from the E. coli CspA gene product (SEQ ID NO: 36). The coding sequences for such chimeric polypeptides are cloned as a fusion with the bacteriophage p3 protein as described, and phage are produced. The resulting phage are screened by proteolysis, and those expressing resistant chimeric protein domains are expanded. The resulting chimeric, folded polypeptides are screened with anti-IgE antibodies to identify those that have chimeric domains closely related to domains on the native IgE. The resulting phage are expanded, the sequences encoding the chimeric domain isolated, and the chimeric domain is expressed on its own (or as a fusion with another carrier protein, optionally with an immunogenic protein, such as another E. coli protein or portion thereof) and the resulting protein is used as an immunogen. A strongly immunogenic carrier can aid in stimulating an immune response specific for the IgE-like chimeric domain. Such a chimeric immunogenic protein is useful as a therapeutic vaccine against asthma or other allergic conditions.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE I
Amino acid sequences and biophysical parameters of de novo proteins.

| Protein | Tm,° C. | ΔG¹ | MW, Da | C-terminal sequence² | |
|---|---|---|---|---|---|
| His-Csp | 59.8 | 3.6 | 8,565 | IQNDGYKSLDEGQKVSFTIES | (SEQ ID NO:16) |
| | | | | | GAKGPAAGNVTSLEA |
| His-Csp/2 | no expr.³ | | 5,854 | WAQAEA | (SEQ ID NO:17) |
| plasmid library: | | | | | |
| His-al | 46.0 | 2.1 | 7,729 | IQNDGYKSLDEGQKVSFTWAQ | (SEQ ID NO:18) |
| | | | | ABA | |
| His-d6 | 47.6 | 1.6 | 10,352 | GSSGFGFITPDDGSKDVFVHFSAIQNYKSLD | (SEQ ID NO:19) |
| | | | | YKSLDEGQKVSFTWAQAEA | |
| genomic library: | | | | | |
| His-1b11 | 57.1 | 2.0 | 10,722 | GSSGAAVRGNPQQGDRVIEGKIKSITDFGIFIGL | (SEQ ID NO:20) |
| | | | | DGGIDGLVHLSDISWAQAEA | |
| His-2f3 | 61.4 | 1.8 | 10,582 | GSSGAGEPEIGAIMLFTAMDGSEMPGVIREING | (SEQ ID NO:21) |
| | | | | DSITVDFNHPPPWAQAEA | |
| His-1c2 | 54.8 | 5.3 | 10,972 | GSSGRVISLTNENGSHSVFSYDALDRLVQQGG | (SEQ ID NO:22) |
| | | | | FDGRTQRYHYDLTWAQAEA | |
| His-1g6 | 48.4 | 2.4 | 10,485 | GSSGKSGVKTDYRASASIACAYAGAGSSDSRR | (SEQ ID NO:23) |
| | | | | SFLCITRSESDGPWAQAEA | |
| ¹The conformational stability ΔG (kcal/mol) at a given temperature T was calculated using the Gibbs-Helmholtz equation ΔG(T) = ΔHm (1-T/Tm) − ΔCp [(Tm-T) + 1n(T/Tm)], while inferring the midpoint of thermal unfolding (Tm) and the enthalpy change for unfolding (ΔHm) at the Tm from the denaturation curve (Agashe & Udgaonkar 1995) and assuming for | | | | | |
| #ΔCp (the difference in heat capacity between unfolded and folded conformation) a value of 12 cal per residue (Edelhoch & Osborne 1976). | | | | | |

²Sequences shown are those following the N-terminal half of CspA, which is MRGSHHHHGSRLQSGKMTGIVKWFNADKGFGFITPDDGSKDVFVHFSA (SEQ ID NO:24).
³The His-(Csp/2) protein was found neither in the soluble nor insoluble fraction of the cytoplasm presumably due to degradation within the cell.

TABLE II
Sequences and origin of genomic segments

| Segment^{a} | Sequence^{d} | | Genetic origin^{e} | Protein origin^{f} |
|---|---|---|---|---|
| 1a7^{b} | GIATSAICDA QVIGEEPGQPTSTT | (SEQ ID NO:25) | 8931 to 9041 in | minus strand |
| | CRFRSK FSAIAFPW | ECAE298, gatC | | |
| 1b11^{b,c} | GAAVRGNPQQ GDRVEGKIKS | (SEQ ID NO:26) | 6382 to 6514 in | 364 to 398 in |
| | ITDFGIFIGL DGGIDGLVHL | ECAE193, rpsA | RS1_ECOLI | |
| | SDISW^{g} | | | |
| 1c2^{b,c} | GRVISLTNEN GSHSVFSYDA | (SEQ ID NO:27) | 2178 to 2303 in | 645 to 686 in |
| | LDRLVQQGGFDGRTQRYHYD | ECAE156, rhsD | RHSD_ECOLI | |
| | LTW | | | |
| 1g6^{b,c} | GKSGVKTDYR ASASIACAYA | (SEQ ID NO:28) | 2694 to 2569 in | frameshift |
| | GAGSSDSRRS FLCITRSESD | ECAE116, rluA | | |
| | GPW | | | |
| 2fl^{b} | GAGTMAEEST DFPGVSRPQD | (SEQ ID NO:29) | 8558 to 8422 in | 452 to 494 in |
| | MGGLGFWYRW NLGH7YIHDTLD | ECAE419, glgB | GLGB_ECOLI | |
| | YMKPHSW | | | |
| 2f3^{b,c} | GAGEPEIGAI MLFTAMDGSE | (SEQ ID NO:30) | 5431 to 5551 in | 89 to 127 in |
| | MPGVIREING DSITVDFNHP | ECAE113, slpA | FKBX_ECOLI | |
| | PPW | | | |
| 2h2^{b} | GSAYNTNGLV QGDKYQIIGF | (SEQ ID NO:31) | 7955 to 7854 in | minus strand |
| | PRFNQLTVYF HNLPW | ECAE475, yjbC | | |
| 3a12^{b} | GKAVGLPEIQ VIRDLFEGLV | (SEQ ID NO:32) | 1479 to 1568 in | 52 to 80 in |
| | NQNEKGEIVP W | ECAE231, b1329 | MPPA_ECOLI | |
| 1g7 | GWLKRKLNLK FNEASIAGCD | (SEQ ID NO:33) | 7290 to 7213 in | frameshift |
| | ALLNAAW | ECAE217,b1191 | | |
| 1h12 | GCVPYTNFSL IYEGKCGMSGGRVE | (SEQ ID NO:34) | 12035 to 11927 in | 334 to 367 in |
| | GKVIYE TQSTHKHSW | ECAE485, cadA | DCLY_ECOLI | |
| 2e2 | GMWPLDMVNA IESGIGGTLGFLAA | (SEQ ID NO:35) | 7398 to 7514 in | 45 to 83 in |
| | VIGPGTILGKIMEVSW | ECAE324, dsdX | DSDX_ECOLI | |
| Segments retaining 80% barstar binding activity after proteolysis of phage in situ^{a} and in solution^{b} and those purified as chimeric proteins^{C}. The sequence of the genomic segment^{d} as a C-terminal appendage to the N-terminal region of CspA (LQSGKMTGIV KWFNADKGFG FITPDDGSKD VFVHFSAGSS; SEQ ID NO:36) is listed; sequences expressed in-frame with the originating gene | | | | |
| # are shown in italics. The location of each segment within the E. coli genome is indicated by nucleotide numbers in the EMBL database entry and name of the originating gene^{e}, and for those expressed in the same frame of the originating gene, the residue numbers of the corresponding protein and its ID in the Swiss protein database are given^{f}. A single base pair deletion after | | | | |
| # the first 29 base pairs in the DNA insert of 1b11 renders the first 10 residues out of frame with the rspA gene^{g}. | | | | |

TABLE III

| | |
|---|---|
| (a) | Amino acid sequences of CspA (SEQ ID NO:37) and His-2f3 (SEQ ID NO:38) |
| | 10 20 30 40 50 |
| CspA | MSGKMTGIVK WFNADKGFGF ITPDDGSKDV FVHFFSAIQND GYKSLDEGQK |
| | * ** |
| 2f3 | ...SGKMTGIVK WFNADKGFGF ITPDDGSKDV FVHFSAGSSG AGE-PEIGAI |
| | 24 34 44 54 63 |
| | 60 70 |
| CspA | VSFTIESGAK GPAAGNVTSL |
| | * * * |
| 2f3 | MLFTAMDGSE MPGVIREING DSITVDFNHP P |
| | 73 83 93 |
| (b) | Folding energy of 2f3 mutants and CspA |

| Protein | ΔG at 298K (kcal/mol) |
|---|---|
| CspA | 3.4 |
| 6H-2f3 | 1.9 |
| 6H-2f3-P58L | 2.8 |
| 6H-213-P58L, A62Q | 6.0 |
| 6H-2f3-P58L, A62Q, A68L | 3.2 |
| (a) The amino acid sequence of CspA is that from the native gene as in the EMPL database. The numbering of the 2f3 sequence takes into account the N-terminal His-tag (MRGSHHHHHHGSRLQ; SEQ ID NO: 39). The C-terminal residues PWAQAEA (SEQ ID NO: 40; compare 2f3 in Table I) were deleted in the constructs used for the data here, as they were partially cleaved in the expressed protein of the | |
| # original His-2f3 construct indicating that they did not participate to the fold of the chimeric domain. Their deletion had no significant effect on the overall folding stability of the domain (1.8 vs. 1.9 kcal/mol in the 2f3 constructs used for data in Table I and III respectively). The residues important for the 13 barrel fold in CspA as discussed in Example 14 are indicated by an | |
| # asterisks. | |

(b) Folding energies were determined as described in Table I. Mutation for 2f3 denote the original amino acid, followed by the residue number and the new amino acids.

TABLE IV
VIRAL ANTIGENS
Retroviral Antigens

| | |
|---|---|
| 1. | HIV Antigens |

| | |
|---|---|
| B. | Hepatitis Viral Antigens |

| | |
|---|---|
| 1. | S, M, and L proteins of hepatitis B virus |
| 2. | Pre-S antigen of hepatitis B |
| 3. | Components of other hepatitis, e.g., hepatitis A, B and C |

| | |
|---|---|
| C. | Influenza Viral Antigens |

| | |
|---|---|
| 1. | Hemagglutinin |
| 2. | Neuraminidase |
| 3. | Other influenza viral components |

| | |
|---|---|
| D. | Measles Viral Antigens |

| | |
|---|---|
| 1. | Measles |
| 2. | Virus fusion protein |
| 3. | Other measles virus components |

| | |
|---|---|
| E. | Rubella Viral Antigens |

| | |
|---|---|
| 1. | Proteins E1 and E2 |
| 2. | Other rubella virus components |

| | |
|---|---|
| F. | Rotaviral Antigens |

| | |
|---|---|
| 1. | VP7sc |
| 2. | Other rotaviral components |

| | |
|---|---|
| G. | Cytomegaloviral Antigens |

| | |
|---|---|
| 1. | Envelope glycoprotein B |
| 2. | Other cytomegaloviral antigen components |

| | |
|---|---|
| H. | Respiratory syncytial viral antigens |

| | |
|---|---|
| 1. | RSV fusion protein |
| 2. | M2 Protein |
| 3. | Other respiratory syncytial viral antigen components |

| | |
|---|---|
| I. | Herpes Simplex Viral Antigens |

| | |
|---|---|
| 1. | Immediate early proteins |
| 2. | Glycoprotein D |
| 3. | Herpes simplex viral antigen components |

| | |
|---|---|
| J. | Varicella zoster viral antigens |

| | |
|---|---|
| 1. | gpI |
| 2. | gpII |
| 3. | other varicella zoster viral antigen components |

| | |
|---|---|
| K. | Japanese Encephalitis Viral Antigens |

| | |
|---|---|
| 1. | Proteins E |
| 2. | M-E |
| 3. | M-E-NS1 |
| 4. | NS1 |
| 5. | NS1-NS2A |
| 6. | 80% E |
| 7. | other japanese encephalitis viral antigen components |

| | |
|---|---|
| L. | Rabies Viral Antigens |

| | |
|---|---|
| 1. | Rabies glycoprotein |
| 2. | Rabies nucleoprotein |
| 3. | Rabies viral antigen components |

| II. | BACTERIAL ANTIGENS | |
|---|---|---|
| | A. | Pertussis bacterial antigens |

| | |
|---|---|
| 1. | Pertussis toxin |
| 2. | Filamentous hemagglutinin |
| 3. | Pertactin |
| 4. | FIM2 |
| 5. | FIM3 |
| 6. | Adenylate cyclase |
| 7. | Other pertussis bacterial antigen components |

| | |
|---|---|
| B. | Diptheria Bacterial Antigens |

| | |
|---|---|
| 1. | Diptherial toxin or toxoid |
| 2. | Other diptheria bacterial antigen components |

| | |
|---|---|
| C. | Tetanus Bacterial Antigens |

| | |
|---|---|
| 1. | Tetanus toxin or toxoid |
| 2. | Other tetanus bacterial antigen components |

| | |
|---|---|
| D. | Streptococcal Bacterial Antigens |

| | |
|---|---|
| 1. | M Proteins |
| 2. | Other streptococcal bacterial antigen components |

| | |
|---|---|
| E. | Gram-Negative Bacilli Bacterial Antigens |

| | |
|---|---|
| 1. | Lipolysaccharides |
| 2. | Other gram-negative bacterial antigen components |

| | |
|---|---|
| F. | Mycobaterium Tuberculosis Bacterial Antigens |

| | |
|---|---|
| 1. | Mycolic acid |
| 2. | Heat shock protein 65 |
| 3. | 30 kDa major secreted protein |
| 4. | antigen 85A |
| 5. | other mycobacterial antigen components |

| | |
|---|---|
| G. | Helicobaxter Pylori Bacterial Antigens |
| H. | Pneumococcal bacterial antigens |

| | |
|---|---|
| 1. | Pneumolysin |
| 2. | Pneumococcal capsular polysaccharides |
| 3. | Other pneumococcal bacterial antigen components |

| | |
|---|---|
| I. | Haemophilus Influenzae Bacterial Antigens |

| | |
|---|---|
| 1. | Capsular polysaccharides |
| 2. | Other haemophilus influenza bacterial antigen |
| | components |

| | |
|---|---|
| J. | Anthrax Bacterial Antigens |

| | |
|---|---|
| 1. | Anthrax protective antigen |
| 2. | Other anthrax bacterial antigen components |

| | |
|---|---|
| K. | Rickettsiae Bacterial Antigens |

| | |
|---|---|
| 1. | RompA |
| 2. | Other rickettsiae bacterial antigen components |
| 3. | Any other bacterial, mycobacterial, mycoplasmal, |
| | rickettsial or chlamydial antigens. |

| III. | FUNGAL ANTIGENS | |
|---|---|---|
| | A. | Candida fungal antigen components |
| | B. | Histoplasma fungal antigens |

| | |
|---|---|
| 1. | Heat shock protein 60 (HSP60) |
| 2. | Other histoplasma fungal antigen components |

| | |
|---|---|
| C. | Cryptococcal Fungal Antigens |

| | |
|---|---|
| 1. | Capsular polysaccharides |
| 2. | Other cryptococcal fungal antigen components |

| | |
|---|---|
| D. | Coccidiodes fungal antigens |

| | |
|---|---|
| 1. | Spherule antigens |
| 2. | Other coccidiodes fungal antigen components |

| | |
|---|---|
| E. | Tinea Fungal Antigens |

| | |
|---|---|
| 1. | Trichophytin |
| 2. | Other coccidiodes fungal antigen components |

| IV. | PROTOZOAL AND OTHER PARASITIC ANTIGENS | |
|---|---|---|
| | A. | Plasmodium Falciparum Antigens |

| | |
|---|---|
| 1. | Merozoite surface antigens |
| 2. | Sporozoite surface antigens |
| 3. | Circumsporozoite antigens |
| 4. | Gametocyte/gamete surface antigens |
| 5. | Blood-stage antigen |
| 6. | 155/RESA |
| 7. | other plasmodial antigen components |

| | |
|---|---|
| B. | Toxoplasma Antigens |

| | |
|---|---|
| 1. | SAG-1 |
| 2. | P30 |
| 3. | Other toxoplasmal antigen components |

| | |
|---|---|
| C. | Schistosomae Antigens |

| | |
|---|---|
| 1. | Glutathione-S-Transferase |
| 2. | Paramyosin |
| 3. | Other schistosomal antigen components |

| | |
|---|---|
| D. | Leishamania Major |

| | |
|---|---|
| 1. | Gp63 |
| 2. | Lipophosphoglycan and its associated protein |
| 3. | Other leishmanial antigen components |

| | |
|---|---|
| E. | Trypansoma Cruzi Antigens |

| | |
|---|---|
| 1. | 75-77 kDa antigen |
| 2. | 56 kDa antigen |
| 3. | other trypanosomal antigen components |

| V. | TUMOR ANTIGENS | |
|---|---|---|
| | A. | Telomerase |
| | B. | Multidrug resistance proteins |
| | C. | MAGE-1 |
| | D. | Alpha fetoprotein |
| | E. | Carcinoembryonic antigen |
| | F. | Mutant p53 |
| | G. | Papillomavirus antigens |
| | H. | Gangliosides of other carbohydrate-containing components of |
| | | melanoma or other tumor cells. |
| VI. | ANTIGENS INVOLVED IN AUTOIMMUNO DISEASES, | |
| | ALLERGY AND GRAFT REJECTION | |
| | A. | Antigens involved in autoimmune diseases |
| | B. | Antigens involved in Diabetes Mellitus |
| | C. | Antigens involved in Arthritis, including those involved in |

| | |
|---|---|
| 1. | Rheumatoid arthritis |
| 2. | Juvenile Rheumatoid arthritis |
| 3. | Osteoarthritis |
| 4. | Psoriatic arthritis |

| | |
|---|---|
| D. | Antigens involved in Multiple Sclerosis |
| E. | Antigens involved in Myasthenia Gravis |
| F. | Antigens involved in Systemic Lupus |
| G. | Antigens involved in Erythematosis |
| H. | Antigens involved in Autoimmune Thyroiditis |
| I. | Antigens involved in Dermatitis, including those involved in |

| | |
|---|---|
| 1. | Atopic dermatitis |
| 2. | Eczematous dermatitis |

| | |
|---|---|
| J. | Antigens involved in Psoriasis |
| K. | Antigens involved in Sjogren's Syndrome, including antigens |
| | involved in |

| | |
|---|---|
| 1. | Keratoconjunctivitis sicca secondary to Sjogren's |
| | Syndrome |

| | |
|---|---|
| L. | Antigens involved in Alopecia Areata |
| M. | Antigens involved in Allergic Responses Due to Arthropod |
| | Bite Reactions |
| N. | Antigens involved in Crohn's Disease |
| O. | Antigens involved in Aphthous Ulcer |
| P. | Antigens involved in ritis |
| Q. | Antigens involved in Conjunctivitis |
| R. | Antigens involved in Keratoconjunctivitis |
| S. | Antigens involved in Ulcerative Colitis |
| T. | Antigens involved in Asthma |
| U. | Antigens involved in Allergic Asthma |
| V. | Antigens involved in Cutaneous Lupus Erythematosus |
| W. | Antigens involved in Scleroderma |
| X. | Antigens involved in Vaginitis |
| Y. | Antigens involved in Proctitis |
| Z. | Antigens involved in Drug Eruptions |
| AA. | Antigens involved in Leprosy Reversal Reactions |
| BB. | Antigens involved in Erythema Nodosum Leprosum |
| CC. | Antigens involved in Autoimmune Uveitis |
| DD. | Antigens involved in Allergic Encephalomyelitis |
| EE. | Antigens involved in Acute Necrotizing Hemorrhagic |
| | Encephalopathy |
| FF. | Antigens involved in Idiopathic Bilateral Progressive |
| | Sensorineural Hearing Loss |
| GG. | Antigens involved in Aplastic Anemia |
| HH. | Antigens involved in Pure Red Cell Anemia |
| II. | Antigens involved in Idiopathic Thrombocytopenia |
| JJ. | Antigens involved in Polychondritis |
| KK. | Antigens involved in Wegener's granulomatosis |
| LL. | Antigens involved in Chronic active hepatitis |
| MM. | Antigens involved in Stevens-Johnson syndrome |
| NN. | Antigens involved in Idopathic sprue |
| OO. | Antigens involved in Lichen planus |
| PP. | Antigens involved in Graves ophthalmopathy |
| QQ. | Antigens involved in Sarcoidosis |
| RR. | Antigens involved in Primary biliary cirrhosis |
| SS. | Antigens involved in Uveitis posterior |
| TT. | Interstitial lung fibrosis |

| | |
|---|---|
| 1. | Glutamic acid decarboxylase 65 (GAD 65) |
| 2. | Native DNA |
| 3. | Myelin basic protein |
| 4. | Myelin proteolipid protein |
| 5. | Acetylcholine receptor components |
| 6. | Thyroglobulin |
| 7. | Thryod stimulating hormone (TSH) receptor |

| | |
|---|---|
| UU. | Antigens involved in allergy |
| VV. | Pollen Antigens |

| | |
|---|---|
| 1. | Japanese cedar pollen antigens |
| 2. | Ragweed pollen antigens |
| 3. | Rye grass pollen antigens |
| 4. | Animal derived antigens |
| 5. | Dust mite antigens |
| 6. | Feline antigens |
| 7. | Histocompatibility antigens |
| 8. | Penicillin and other therapeutic drugs. |

| | |
|---|---|
| WW. | Antigens involved in graft rejection |

| | |
|---|---|
| 1. | Antigenic components of the graft to be transplanted |
| 2. | Heart, lung liver, pancreas, kidney and neutral graft |
| | components. |

### REFERENCES

Ausubel, F. M., Brent, R., Kingston, R. E., Moore, D. D., Seidman, J. G., Smith, J. A. & Struhl, K. (1995) Current protcols in molecular biology. Chapter 2.4.1. Wiley & Sons.

Agashe, V. R. & Udgaonkar, J. B. (1995) Thermodynamics of denaturation of barstar: evidence for cold denaturation and evaluation of the interaction with guanidine hydrochloride. Biochemistry 34, 3286-3299.

Alba, E. de, Santoro, J., Rico, M. & Jimenez, M. A. (1999) De novo design of a monomeric three-stranded anti-parallel β-sheet. Protein Sci. 8, 854-865.

Altschul, S. F., Madden, T. L., Schaffer, A. A., Zhang, J. H., Zhang, Z., Miller, W. & Lipman, D. J. (1997). Gapped BLAST and PSI-BLAST: a new generation of protein database search programs. Nucleic Acids Res. 25, 3389-3402.

Barbas, C. F., Crowe, J. E., Cababa, D., Jones, T. M., Zebedee, S. L., Murphy, B. R., Chanock, R. M. & Burton D. R. (1992) Human monoclonal fab fragments derived from a combinatorial library bind to respiratory syncytial virus-f glycoprotein and neutralize infectivity. Proc. Natl. Acad. Sci. USA 89, 10164-10168.

Bogarad, L., & Deem, M. (1999) A hierarchical approach to protein molecular evolution. Proc. Natl. Acad. Sci. USA 96, 2591-2595.

Breitling, F., Dubel, S., Seehaus, T., Klewinghaus, I. & Little M. (1991) A surface expression vector for antibody screening. Gene 104, 147-153.

Burton, D. R., Barbas, C. F., Persson, M. A. A., Koenig, S., Chanock, R. M. & Lemer, R. A. (1991) A large array of human monoclonal-antibodies to type-1 human-immunodeficiency-virus from combinatorial libraries of asymptomatic seropositive individuals. Proc. Natl. Acad. Sci. USA 88, 10134-10137.

Bycroft, M., Hubbard, T. J., Proctor, M., Freund, S. M. & Murzin, A. G. (1997) The solution structure of the s1 RNA binding domain: a member of an ancient nucleic acid-binding fold. Cell 88, 235-242.

Caton, A. J. & Koprowski, H. (1990) Influenza-virus hemagglutinin-specific antibodies isolated from a combinatorial expression library are closely related to the immune-response of the donor. Proc. Natl. Acad. Sci. USA 87, 6450-6454.

Chang, C. N., Landolfi, N. F. & Queen, C. (1991) Expression of antibody fab domains on bacteriophage surfaces—potential use for antibody selection. J. Immunol. 147, 3610-3614.

Clackson, T., Hoogenboom, H. R., Griffiths, A. D. & Winter, G. (1991) Making antibody fragments using phage display libraries. Nature 352, 624-628.

Cooper, J. A., Hayman, W., Reed, C., Kagawa, H., Good, M. F. & Saul, A. (1997) Mapping of conformational B cell epitopes within alpha-helical Coiled coil proteins. Mol. Immunol. 34, 433-440

Davies, J. & Riechmann, L. (1995) An antibody VH domain with a lox-Cre site integrated into its coding region: bacterial recombination within a single polypeptide chain. FEBS Lett. 377, 92-96.

Davidson, A. R. & Sauer, R. T. (1994) Folded proteins occur frequently in libraries of random amino-acid-sequences. Proc. Natl. Acad. Sci. USA 91, 2146-2150.

Devereux, J., Haeberlie, P. & Smithies O. (1984) A comprehensive set of sequence analysis program for the VAX. Nucl. Acids Res. 12, 387-395.

Dower, W. J. & Fodor, S. P. A. (1991) The search for molecular diversity. 2. recombinant and synthetic randomized peptide libraries. Annu Rep Med Chem 26, 271-280.

Edelhoch, H. & Osborne, J. C., Jr. (1976) The thermodynamic basis of the stability of proteins, nucleic acids, and membranes. Adv. Prot. Chem. 30, 183-250.

Eggertsson, G. & Söll, D. (1988) Transfer ribonucleic acid-mediated suppression of termination codons in Escherichia Coli. Microbiol. Rev. 52, 354-374.

Feng, D. F. & Dolittle, R. F. (1987) Progressive sequence alignment as a prerequisite to correct phylogenetic trees. J. of Molec. Evol. 25, 351-360.

Fincuane, M. D., Tuna, M., Lees, J. H. & Woolfson, D. N. (1999) Core-directed protein design. I. An experimental method for selecting stable proteins from combinatorial libraries. Biochemistry 38, 11604-11612.

Fire, A. & Xu, S. Q. (1995) Rolling replication of short DNA circles. Proc. Natl. Acad. Sci. USA 92, 4641-4645.

Fodor. S. P. A., Read, J. L., Pirrung, M. C., Stryer, L., Lu, A. T. & Solas, D. (1991) Light-directed, spatially addressable parallel chemical synthesis. Science 251, 767-773.

Fontana, A., Laureto, P. de, Filipis, V. de, Scaramella, E. & Zambonin, M. (1997) Probing the partly folded states of proteins by limited proteolysis. Fold. Des. 2, R17-R26.

Gibson, T. J. (1984) Ph. D. Thesis, University of Cambridge, UK.

Goldstein, J., Pollitt, N. S. & Inouye, M. (1990) Major cold shock protein of Escherichia coli. Proc. Natl. Acad. Sci. USA 87, 283-287.

Greenfield, N & Fasman, G. D. (1969) Computed circular dichroism spectra for the evaluation of protein conformation. Biochemistry 8, 4108-4116.

Hardies, S. C., Hillen, W., Goodman, T. C. & Wells, R. D. (1979) High resolution thermal denaturation analyses of small sequenced DNA restriction fragments containing Escherichia coli lactose genetic control loci. J. Biol. Chem. 254, 5527-5534.

Hartley, R. W. (1993) Directed mutagenesis and barnase-barstar recognition. Biochemistry 32, 5978-5984.

Hawkins, R. E., Russell, S. J. & Winter, G. (1992) Selection of phage antibodies by binding-affinity—mimicking affinity maturation. J. Mol. Biol. 226, 889-896.

Hawkins, R. E. & Winter. G. (1992) Cell selection-strategies for making antibodies from variable gene libraries—trapping the memory pool. Eur. J. Immunol. 22, 867-870.

Hecht, M. (1994) De novo design of β-sheet proteins. Proc. Natl. Acad. Sci. USA 91, 8729-8730.

Higgins, D. G. & Sharp, P. M. (1989) Fast and sensitive multiple sequence alignment on a microcomputer. CABIOS 5, 151-153.

Hoogenboom, H. R., Griffiths, A. D., Johnson, K. S., Chiswell, D. J., Hudson, P. & Winter, G. (1991) Multi-subunit proteins on the surface of filamentous phage: methodologies for displaying antibody (Fab) heavy and light chains. Nucleic Acids Res. 19, 4133-4137.

Hubbard, S. J., Eisenmenger, F. & Thornton, J. M. (1994) Modeling studies of the change in conformation required for cleavage of limited proteolytic sites. Protein Science 3, 757-768.

Huse, W. D., Sastry, L., Iverson, S. A., Kang, A. S., Altingmees, M., Burton, D. R., Benkovic, S. J. & Lerner, R. A. (1989) Generation of a large combinatorial library of the immunoglobulin repertoire in phage-lambda. Science 246 1275-1281.

Hutchens, T. W., and Yip, T-T. (1993) New desorption strategies for the mass spectrometric analysis of macromolecules. Rapid Commun. Mass Spectrom. 7, 576-580.

Hutchison III, C. A., Phillips, S., Edgell, M. H., Gillam, S., Jahnke, P. & Smith, M. (1978) Mutagenesis at a specific position in a DNA sequence. J. Biol. Chem. 253, 6551-6560.

Jiang, W. N., Hou, Y. & Inouye, M. (1997) CspA, the major cold-shock protein of Escherichia coli, is an RNA chaperone. J. Biol. Chem. 272, 196-202.

Johnson, W. C. Jr (1990) Protein secondary structure and circular-dichroism—a practical guide. Proteins 7, 205-214.

Jones, B. E., Jennings, P. A., Pierre, R. A. & Matthews, C. R. (1994) Development of nonpolar surfaces in the folding of Escherichia coli dihydrofolate reductase detected by 1-anilinonaphthalene-8-sulfonate binding. Biochemistry 33, 15250-15258.

Kamtekar, S., Schiffer, J. M., Xiong, H., Babik, J. M. & Hecht, M. (1993) Protein design by binary patterning of polar and nonpolar amino acids. Science 262, 1680-1685.

Kang, A. S., Jones, T. M. & Burton, D. R. (1991) Antibody redesign by chain shuffling from random combinatorial immunoglobulin libraries. Proc. Natl. Acad. Sci. USA 88, 11120-11123.

Kristensen, P. & Winter, G. (1997) Proteolytic selection for protein folding using filamentous bacteriophages. Folding Des. 3, 321-328.

Kortemme, T., Ramirez-Alvardo, M. & Serrano, L. (1998) Design of a 20-amino acid, three-starnded β-sheet protein. Science 281, 253-256.

Kuttler, C., Nussbaum, A. K., Dick, T. P., Rammensee, H. G., Schild, H. & Hadeler, K. P. (2000) An algorithm for the prediction of proteosomal cleavages. J. Mol. Biol. 298, 417-420.

Lerner, R. A., Kang, A. S., Bain, J. D., Burton, D. R. & Barbas, C. F. (1992) Antibodies without immunization. Science 258, 1313-1314.

Liljeqvist, S. & Stahl, S. (1999) Production of recombinant subunuit vaccines: protein immunogens, live delivery systems and nucleic acid vaccines. J. Biotechnol. 73, 1-33.

Low, N. M., Holliger, P. & Winter, G. (1996) Mimicking somatic hypermutation: Affinity maturation of antibodies displayed on bacteriophage using a bacterial. J. Mol. Biol. 260, 359-368.

Lowman H B, Bass S H, Simpson N. & Wells, J. A. (1991) Selecting high-affinity binding-proteins by monovalent phage display. Biochemistry 30, 10832-10838.

Lubienski, M. J., Bycroft, M., Jones, D. N. M. & Fersht, A. R. (1993) Assignment of the backbone H-1 and N-15 NMR resonances and secondary structure characterisation of barstar. FEBS Lett. 332, 81-87.

Marks, J. D., Hoogenboom, H. R., Bonnert, T. P., McCafferty, J., Griffiths, A. D. & Winter, G. (1991) By-passing immunization—human-antibodies from V-gene libraries displayed on phage. J. Mol. Biol. 222, 581-597.

Marks, J. D., Hoogenboom H R, Griffiths A D, Winter G (1992) Molecular evolution of proteins on filamentous phage—mimicking the strategy of the immune-system. J. Biol. Chem. 267, 16007-16010.

McCafferty, J., Griffiths, A. D., Winter, G. & Chiswell, D. J. (1990) Phage antibodies—filamentous phage displaying antibody variable domains. Nature 348, 552-554.

Meiering, E. M., Serrano, L. & Fersht, A. R. (1992) Effect of active site residues in bamase on activity and stability. J. Mol. Biol. 225, 585-589.

Mullinax, R. L., Gross, E. A., Amberg, J. R., Hay, B. N., Hogrefe, H. H., Kubitz, M. M., Greener, A., Altingmees, M., Ardourel, D., Short, J. M., Sorge, J. A. & Shopes, B. (1990) Identification of human-antibody fragment clones specific for tetanus toxoid in a bacteriophage-lambda immunoexpression library. Proc. Natl. Acad. Sci. USA 87, 8095-8099.

Murzin A. G., Brenner S. E., Hubbard T. & Chothia C. (1995). SCOP: a structural classification of proteins database for the investigation of sequences and structures. J. Mol. Biol. 247, 536-540.

Myers, E. W. & Miller, W. (1988) “Optimal Alignments in Linear Space”, CABIOS 4, 11-17.

Needleman, S. B. & Wunsch, C. D. (1970) A general method-applicable to the search for similarities in the amino acid sequences of two proteins. J. Mol. Biol. 48, 444-453.

Newkirk, K., Feng, W. Q., Jiang, W. N., et al.(1994) Solution nmr structure of the major cold shock protein (cspa) from escherichia-coli—identification of a binding epitope for DNA. Proc. Natl. Acad. Sci. USA 91, 5114-5118.

Pace, C. N. (1990) Conformational stability of globular proteins. Trends Biochem. Sci. 15, 14-17.

Persson, M. A. A., Caothien, R. H. & Burton, D. R. (1991) Generation of diverse high-affinity human monoclonal-antibodies by repertoire cloning. Proc. Natl. Acad. Sci. USA 88, 2432-2436.

Piotto, M, Saudek, V. & Sklenar, V. (1992) Gradient-tailored excitation for single-quantum nmr-spectroscopy of aqueous-solutions. J. Biomolecular NMR 2, 661-665.

Quinn, T. P., Tweedy, N. B., Williams, R. W., Richardson, J. S. & Richardson, D. C. (1994) Betadoublet: De novo design, synthesis, and characterisation of a β-sandwich protein. Proc. Natl. Acad. Sci. USA 91, 8747-8751.

Rakonjac, J., Jovanovic, G. & Model, P. (1997) Filamentous phage infection-mediated gene expression: construction and propagation of the gIII deletion mutant helper phage R408d3. Gene 198, 99-103.

Regan, L. (1998) Proteins to order? Structure 6, 1-4.

Riechmann, L. & Davies, J. (1995) Backbone assignment, secondary structure and Protein A binding of an isolated, human antibody VH domain. J. Biomol. NMR 6, 141-152.

Riechmann, L. & Holliger, P. (1997) The C-terminal doamin of TolA is the coreceptor for filamentous phage infection of E. coli. Cell 90, 351-360.

Riechmann, L., & Weill, M. (1993) Phage display and selection of a site-directed randomized single-chain antibody Fv fragment for its affinity improvement. Biochemistry 32, 8848-8855.

Sauer, R. T. (1996) Protein folding from a combinatorial perspective. Folding Des. 1, R27-R30.

Schindelin, H., Maraheil, M. A. & Heinemann, U. (1994) Crystal structure of CspA, the major cold shock protein of Escherichia coli. Proc. Natl. Acad. Sci. USA 91, 5119-5123.

Schroder, K., Graumann, P., Schnuchel, A., Holak, T. A. & Marahiel, M. A. (1995) Mutational analysis of the putative nucleic acid-binding surface of the cold-shock domain, CspB, revealed an essential role of aromatic and basic residues in binding of single-stranded-DNA containing the y-box motif. Mol. Microbiol. 16, 699-708.

Scott, J. K. & Smith, G. P. (1990) Searching for peptide ligands with an epitope library. Science 249, 386-390.

Sieber, V., Plueckthun, A. & Schmid, F. X. (1998) Selecting proteins with improved stability by a phage-based method. Nat. Biotechnol. 16, 955-960.

Smith, T. F. & Waterman, M. S. (1981) Comparison of Bio-sequences. Advances in Applied Mathematics 2, 482-489.

Smith, T. F., Waterman, M. S. & Sadler, J. R. (1983) Statistical characterisation of nucleic acid sequence functional domains. Nucleic Acids Res. 11, 2205-2220.

Stemberg, N. & Hamilton, D. (1981) Bacteriophage PI site-specific recombination. I. Recombination between loxP sites. J. Mol. Biol., 150, 467-486.

Tame J. R., Murshudov, G. N., Dodson, E. J., Neil, T. K., Dodson, G. G., Higgins, C. F. & Wilkinson, A. J. (1994) The structural basis of sequence-independent peptide binding by OppA protein. Science 264, 1578-1581.

Thompson, J. D., Higgins, D. G. & Gibson, T. J. (1994) ClusterW: improving the sensitivity of progressive multiple sequence alignment through sequence weighing, positions-specific gap penalties and weight matrix choice. Nucleic Acid Res. 22, 4673.

Wilbur, W. J. & Lipman, D. J. (1983) Rapid similarity searches of nucleic-acid and protein data banks. Proc. Natl. Acad. Sci. USA 80, 726-730.

Wütthrich, K. (1986) NMR of proteins and nucleic acids. Chapter 3. Wiley & Sons.

Zacher, A. N., Stock, C. A., Golden, J. W. & Smith, G. P. (1980) A new filamentous phage cloning vector: fd-tet. Gene 9, 127-140.

Cited patents and patent applications:

EPO₃₂₂₅₃₃B

PCT/GBOO/00030

PCT/GB98/01889

WO84/03564

WO88/08453

WO91/05058

WO90/05785

WO90/07003

WO90/15070

WO91/02076

WO92/00091

WO92/02536

WO92/10092

WO93/06121

WO95/11922

WO95/22625

U.S. Pat. No. 4,631,211

U.S. Pat. No. 5,143,854

U.S. Pat. No. 6,174,528

1. A pharmaceutical composition comprising a chimeric, folded protein domain and a pharmaceutically acceptable carrier, wherein said chimeric folded protein domain is selected from a repertoire of chimeric protein domains, said domain comprising two or more sequence segments from parent amino acid sequences that are non-homologous, wherein said two or more sequence segments do not form stable folds in isolation from said parent amino acid sequences.

2. The pharmaceutical composition of claim 1, wherein said sequence segments do not consist of complete protein domains, and wherein said sequence segments do not consist solely of single and complete protein structural elements.

3. The pharmaceutical composition of claim 1, wherein said two or more sequence segments are combined non-covalently.

4. The pharmaceutical composition of claim 1, wherein at least one of the parent amino acid sequences is from a protein.

5. The pharmaceutical composition of claim 3, wherein at least one of the parent amino acid sequences is from a protein selected from the group consisting of a naturally occurring protein, an engineered protein, a protein with a known binding activity, a protein with a known binding activity for an organic compound, a protein with a known binding activity for a peptide or polypeptide, a protein with a known binding activity for a carbohydrate, a protein with a known binding activity for a nucleic acid, a known binding activity for a hapten, a protein with a known binding activity for a steroid, a protein with a known binding activity for an inorganic compound, and a protein with an enzymatic activity.

6. The pharmaceutical composition of claim 1, wherein the parent amino acid sequences are from the open reading frames of a single genome, and:
(a) said reading frames are those used in nature; or
(b) said reading frames are not those used in nature.

7. The pharmaceutical composition of claim 1, wherein the parent amino acid sequences are derived from the open reading frames of two or more genomes, and
(a) said reading frames are those used in nature; or
(b) said reading frames are not those used in nature.

8. The pharmaceutical composition of claim 1, wherein said chimeric, folded protein domain is resistant to in vivo or in vitro proteolysis by protease enzymes.

9. The pharmaceutical composition of claim 1, wherein the sequence segments are from parent domains with the same polypeptide fold in their structures.

10. The pharmaceutical composition of claim 1, wherein the sequence segments are from parent domains with different polypeptide folds in their structures.

11. The pharmaceutical composition of claim 1, wherein said chimeric, folded protein domain has a free energy of folding greater than 1.6 kcal/mol.

12. The pharmaceutical composition of claim 1, wherein said chimeric, folded protein domain has a free energy of folding greater than 3 kcal/mol.

13. The pharmaceutical composition of claim 1, wherein said chimeric, folded protein domain has a free energy of folding greater than 5 kcal/mol.

14. The pharmaceutical composition of claim 1, wherein one or more of said sequence segments is fused to one or more additional and complete protein domains.

15. The pharmaceutical composition of claim 1, wherein said chimeric, folded protein domain is fused to the coat protein of a filamentous bacteriophage, said bacteriophage encapsidating a nucleic acid encoding said protein domain.

16. The pharmaceutical composition of claim 1, wherein a single sequence segment is from a human protein.

17. The pharmaceutical composition of claim 1, wherein two or more sequence segments are from human proteins.

18. The pharmaceutical composition of claim 16 or 17, wherein at least one of said sequence segments is from a source other than a human protein.

19. The pharmaceutical composition of claim 16 or 17, wherein all sequence segments are from human proteins.

20. The pharmaceutical composition of claim 1, wherein said chimeric, folded protein domain comprises a B cell epitope of at least one of the parent amino acid sequences.

21. The pharmaceutical composition of claim 20, wherein said chimeric, folded protein domain comprises a conformational B cell epitope of at least one of said parent amino acid sequences.

22. The pharmaceutical composition of claim 20, wherein said chimeric, folded protein domain comprises a conformational B cell epitope of at least one of said parent amino acid sequences and at least one T cell epitope.

23. The pharmaceutical composition of claim 22, wherein said conformational B cell epitope and at least one T cell epitope are derived from the same amino acid sequence.

24. The pharmaceutical composition of claim 1, wherein said chimeric, folded protein domain cross reacts with antibodies raised against a parent amino acid sequence.

25. The pharmaceutical composition of claim 24, wherein said chimeric, folded protein domain cross reacts with antibodies raised against the folded parent protein of one of said sequence segments.

26. The pharmaceutical composition of claim 24, wherein said chimeric, folded protein domain cross reacts with antibodies specific for the folded parent protein of one of said sequence segments, but not with antibodies specific for the unfolded parent protein or fragments thereof.

27. The pharmaceutical composition of claim 1 wherein the amino acid sequences of said chimeric, folded protein domain are altered to increase stability or function of the chimeric protein.

28. The pharmaceutical composition of claim 1, wherein said chimeric, folded protein domain comprises at least one reaction group for covalent linkage.

29. The pharmaceutical composition of claim 1, wherein said chimeric, folded protein domain comprises at least one reaction group for non-covalent linkage.

30. The pharmaceutical composition of claim 1, wherein said chimeric, folded protein domain comprises at least one D-amino acid.

31. The pharmaceutical composition of claim 1, wherein said chimeric, folded protein domain comprises at lease one non-naturally occurring amino acid.

32. The pharmaceutical composition of claim 1, wherein said chimeric, folded protein domain comprises at least one amino acid having a label or a tag.

33. A pharmaceutical composition comprising a pharmaceutically acceptable carrier and a chimeric, folded protein domain selected from a repertoire of chimeric folded proteins, said protein domain comprising two or more sequence segments from parent amino acid sequences wherein each of said segments in said chimeric protein domain comprises a region of common sequence, and in which said region of common sequence does not consist solely of one or more complete structural elements.

34. The pharmaceutical composition of claim 33, wherein said region of common sequence is at least 10 identical contiguous amino acid residues in length.

35. The pharmaceutical composition of claim 34, wherein said region of common sequence is at least 20 identical contiguous amino acid residues in length.

36. A pharmaceutical composition comprising a pharmaceutically acceptable carrier and a chimeric, folded protein domain selected from a repertoire of chimeric folded proteins comprising two or more sequence segments, wherein each of said segments:
(a) is from parent proteins with a common fold; and
(b) comprises a common region of the common fold and in which said common region of the common fold does not consist of one or more complete structural elements.

37. The chimeric, folded protein domain of claim 36 wherein each of said segments is from different proteins which are homologous in sequence.

38. The pharmaceutical composition of claim 36 wherein each of said segments is from the same protein.

39. The pharmaceutical composition of claim 36 in which the common region of the common fold is at least 10 contiguous amino acid residues in length.

40. The pharmaceutical composition of claim 36 in which the common region of the common fold is at least 20 contiguous amino acid residues in length.

41. The pharmaceutical composition of claim 36, in which the amino acid sequences of said parent proteins are from the open reading frames of a genome, wherein said reading frames are the natural reading frame of the genes encoded by said genome.

42. The pharmaceutical composition of claim 33 wherein said chimeric, folded protein domain is resistant to in vivo or in vitro proteolysis by protease enzymes.

43. The pharmaceutical composition of claim 33, wherein said chimeric, folded protein domain has a free energy of folding greater than 1.6 kcal/mol.

44. The pharmaceutical composition of claim 33, wherein one or more of the sequence segments of said chimeric, folded protein is fused to one or more additional and complete protein domains.

45. The pharmaceutical composition of claim 33, wherein said chimeric, folded protein domain fused to the coat protein on a filamentous bacteriophage, said bacteriophage encapsidating a nucleic acid encoding said protein domain.

46. The pharmaceutical composition of claim 33, wherein a single sequence segment of said chimeric, folded protein domain is from a human protein.

47. The pharmaceutical composition of claim 33, wherein two or more of the sequence segments of said chimeric, folded protein domain are from a human protein.

48. The pharmaceutical composition of claim 33, wherein at least one of the segments of said chimeric, folded protein domain is not from a human protein.

49. The pharmaceutical composition of claim 33, wherein all segments of said chimeric, folded protein domain are from human proteins.

50. The pharmaceutical composition of claim 33, wherein said chimeric, folded protein domain comprises a B cell epitope of at least one of the parent amino acid sequences.

51. The pharmaceutical composition of claim 50, wherein said B cell epitope is a confirmational epitope of at least one of the parent amino acid sequences.

52. The pharmaceutical composition of claim 50, wherein said chimeric, folded protein domain comprises a conformational B cell epitope of at least one of the parent amino acid sequences and at least one T cell epitope.

53. The pharmaceutical composition of claim 50 wherein said chimeric, folded protein domain comprises a conformational B cell epitope of at least one of the parent amino acid sequence and at least one T cell epitope, and wherein said epitopes are derived from the same parent amino acid sequence.

54. The pharmaceutical composition of claim 33 wherein said chimeric, folded protein domain cross reacts with antibodies raised against a parent amino acid sequence.

55. The pharmaceutical composition of claim 33 wherein said chimeric, folded protein domain cross reacts with antibodies raised against the folded parent protein.

56. The pharmaceutical composition of claim 33, wherein said chimeric, folded protein domain cross reacts with antibodies raised against the folded parent protein, but not with antibodies specific for the unfolded parent protein or fragments thereof.

57. The pharmaceutical composition of claim 33, wherein the amino acid sequences of said chimeric, folded protein domain are altered to increase stability or function of the chimeric protein.

58. A pharmaceutical composition of claim 32, wherein said chimeric, folded protein domain comprises at least one reaction group for covalent linkage.

59. The pharmaceutical composition of claim 33, wherein said chimeric, folded protein domain comprises at least one reaction group for non-covalent linkage.

60. The pharmaceutical composition of claim 33, wherein said chimeric, folded protein domain comprises at least one D-amino acid.

61. The pharmaceutical composition of claim 33, wherein said chimeric, folded protein domain comprises at least one non-naturally-occurring amino acid.

62. The pharmaceutical composition of claim 33, wherein said chimeric, folded protein domain comprises at least one amino acid having a label or a tag.

63. A method for preparing a pharmaceutical composition of claim 1, comprising the steps of:
(a) providing a first library of nucleic acids, said library comprising coding sequences encoding sequence segments from one or more amino acid sequences;
(b) providing a second library of nucleic acids, said library comprising coding sequences encoding sequence segments derived from one or more amino acid sequences;
(c) combining the coding sequences to form a combinatorial library of nucleic acids, said nucleic acids comprising contiguous coding sequences encoding sequence fragments derived from the first and second libraries;
(d) transcribing and/or translating the contiguous coding sequences to produce the encoded protein domains;
(e) selecting a chimeric protein domain which adopts a folded structure; and
(f) combining said chimeric, folded protein domain with a pharmaceutically acceptable carrier.

64. The method of claim 63, further comprising the steps of:
(i) analysing the sequence of the selected chimeric protein domains to identify the parent amino acid sequences of said sequence segments; and
(ii) comparing the sequences of each of said parent amino acid sequences to determine whether said parent amino acid sequences are non-homologous.

65. A method for preparing a pharmaceutical composition of claim 1 wherein said chimeric, folded protein domain comprises two or more sequence segments derived from parent amino acid sequences that are non-homologous, wherein said two or more sequence segments do not form stable folds in isolation from said parent amino acid sequences, and wherein the sequence segments are from parent domains with different polypeptide folds in the structure, said method comprising the steps of claim 63 or 64 and the additional step of comparing the structures of each of said parent amino acid sequences to identify whether they have the same polypeptide folds.

66. The method of claim 63, wherein steps (b) and (c) are modified as follows:
(b) providing a partner coding sequence encoding a sequence segment derived from one protein;
(c) combining the library of step (a) and the partner coding sequence of step (b) to form a combinatorial library of nucleic acids, said nucleic acids comprising contiguous coding sequences encoding sequence fragments from the first library, joined to said partner coding sequence.

67. The method of claim 63, wherein the domains which adopt a folded structure are selected by one or more methods selected from the group consisting of in vivo proteolysis, in vitro proteolysis, binding ability, functional activity and expression.

68. The method of claim 67, wherein said binding ability is to an antibody raised against a parent protein.

69. A method for preparing a pharmaceutical composition of claim 27, wherein said sequence segments of said parent amino acid sequences are altered subsequent to their juxtaposition in said chimeric protein domain, comprising a step selected from the group consisting of:
(a) preselecting and introducing specific or random mutations at predefined positions within the gene of the chimeric protein;
(b) deleting nucleotides within the gene of the chimeric protein so as to delete amino acid residues;
(c) inserting nucleotides within the gene of the chimeric protein so as to insert amino acid residues; (d) appending nucleotides to the gene of the chimeric protein so as to append amino acid residues;
(e) randomly introducing mutations in all or part of the gene encoding the chimeric protein through recombinant DNA technology;
(f) randomly introducing mutations in the gene of the chimeric protein through propagation in mutator cells;
(g) introducing derivatives of natural amino acid during chemical synthesis;
(h) chemically derivatizing amino acid groups after synthesis;
(i) multimerizing the chimeric proteins through concatenation of two or more copies of the gene in a single open reading frame;
(j) multimerizing the chimeric proteins through covalent linkage of two or more copies of the chimeric protein domain after translation; and
(k) multimerizing the chimeric proteins through fusion to a multimeric partner.

70. A method for preparing a pharmaceutical composition of claim 33, comprising the steps of:
(a) providing a first library of nucleic acids, said library comprising coding sequences encoding sequence segments from one or more amino acid sequences;
(b) providing a second library of nucleic acids, said library comprising coding sequences encoding sequence segments from one or more amino acid sequences;
(c) combining the coding sequences to form a combinatorial library of nucleic acids, said nucleic acids comprising contiguous coding sequences encoding sequence fragments derived from the first and second libraries;
(d) transcribing and/or translating the contiguous coding sequences to produce the encoded protein domains; and
(e) selecting the chimeric protein domains which adopt a folded structure;
(f) combining said chimeric, folded protein domain with a pharmaceutically acceptable carrier.

71. The method of claim 70, further comprising the steps of:
(i) analysing the sequence of the selected chimeric protein domains to identify the parent amino acid sequences of said sequence segments; and
(ii) comparing said parent amino acid sequences to determine whether they comprise common sequences that do not consist solely of one or more complete structural elements.

72. The method for preparing a chimeric, folded protein domain of claim 36, comprising the steps of:
(a) providing a first library of nucleic acids, said library comprising coding sequences encoding sequence segments from one or more amino acid sequences;
(b) providing a second library of nucleic acids, said library comprising coding sequences encoding sequence segments from one or more amino acid sequences;
(c) combining the coding sequences to form a combinatorial library of nucleic acids, said nucleic acids comprising contiguous coding sequences encoding sequence fragments from the first and second libraries;
(d) transcribing and/or translating the contiguous coding sequences to produce the encoded protein domains;
(e) selecting the chimeric protein domains, which adopt a folded structure;
(f) analysing the sequence of the selected chimeric protein domains to identify the parent amino acid sequences of said sequence segments;
(g) comparing the structures of the parent amino acid sequences to determine whether the parent amino acid sequences have a common fold; and
(h) identifying a selected chimeric protein domain wherein the segments comprise a common region of the common fold;
(i) combining chimeric, folded protein domain of step (h) with a pharmaceutically acceptable carrier.

73. The method of claim 70, wherein step (b) and (c) are modified such that:
(b) providing a partner coding sequence encoding a sequence segment from one protein;
(c) combining the library and partner coding sequence to form a combinatorial library of nucleic acids, said nucleic acids comprising contiguous coding sequences encoding sequence fragments from the first library and the partner coding sequence.

74. The method of claim 70 wherein the domains which adopt a folded structure are selected by one or more methods selected from the group consisting of in vivo proteolysis, in vitro proteolysis, binding ability, functional activity and expression.

75. The method of claim 74, wherein said binding ability is to an antibody raised against a parent protein

76. A method for preparing a pharmaceutical composition of claim 33, wherein the sequence segments of the parent amino acid sequences are altered subsequent to their juxtaposition using a step selected from the group consisting of:
(a) preselecting and introducing specific or random mutations at predefined positions within the gene of the chimeric protein;
(b) deleting nucleotides within the gene of the chimeric protein so as to delete amino acid residues;
(c) inserting nucleotides within the gene of the chimeric protein so as to insert amino acid residues;
(d) appending nucleotides to the gene of the chimeric protein so as to append amino acid residues;
(e) randomly introducing mutations in all or part of the gene encoding the chimeric protein through recombinant DNA technology;
(f) randomly introducing mutations in the gene of the chimeric protein through propagation in mutator cells;
(g) introducing derivatives of natural amino acid during chemical synthesis;
(h) chemically derivatising amino acid groups after synthesis;
(i) multimerising the chimeric proteins through concatenation of two or more copies of the gene in a single open reading frame;
(j) multimerising the chimeric proteins through covalent linkage of two or more copies of the chimeric protein domain after translation;
(k) multimerising the chimeric proteins through fusion to a multimeric partner.

77. A method of raising, in an individual, an immune response against one or more of the parent amino acid sequences from which the sequence segments of a chimeric, folded protein domain of a pharmaceutical composition of claim 1 are taken, the method comprising administering an effective amount of a pharmaceutical composition of claim 1 to said individual.

78. A method of raising, in an individual, an immune response against one or more of the parent amino acid sequences from which the sequence segments of a chimeric, folded protein domain of a pharmaceutical composition of claim 33 are taken, the method comprising administering an effective amount of a pharmaceutical composition of claim 33 to said individual.

79. A method of raising, in an individual, an immune response against one or more of the parent amino acid sequences from which the sequence segments of a chimeric, folded protein domain of a pharmaceutical composition of claim 36 are taken, the method comprising administering an effective amount of a pharmaceutical composition of claim 36 to said individual.
